SEQUENCE LISTING (668 sequences)

SEQ ID NO: 1 - DNA, 26 bp, Artificial Sequence
  DQA5′ PCR primer
tcttatggtgtaaacttgtaccagtc

SEQ ID NO: 2 - DNA, 26 bp, Artificial Sequence
  DQA3′ PCR primer
tcttatggtgtaaacttgtaccagtc

SEQ ID NO: 3 - DNA, 22 bp, Artificial Sequence
  DQB5′ PCR primer
gcgtgcgtcttgtgagcagaag

SEQ ID NO: 4 - DNA, 21 bp, Artificial Sequence
  DQB3′ PCR primer
cctgtccaccgccgcccgttt

SEQ ID NO: 5 - protein, 12 aa, Artificial Sequence
  DQ2 alpha-I epitope
QLQPFPQPELPY

SEQ ID NO: 6 - protein, 11 aa, Artificial Sequence
  DQ2 alpha-II epitope
PQPELPYPQPE

SEQ ID NO: 7 - protein, 12 aa, Homo sapiens
ASSIRSTDTQYF

SEQ ID NO: 8 - protein, 14 aa, Homo sapiens
ASSLGQPSTDTQYF

SEQ ID NO: 9 - protein, 14 aa, Homo sapiens
ASSIRTSGAHEQYF

SEQ ID NO: 10 - protein, 14 aa, Homo sapiens
ASSLRAGGSHEQYF

SEQ ID NO: 11 - protein, 14 aa, Homo sapiens
ASSFQRGAADTQYF

SEQ ID NO: 12 - protein, 14 aa, Homo sapiens
ASSFRVGAVDTQYF

SEQ ID NO: 13 - protein, 12 aa, Homo sapiens
ASSIRWTDTQYF

SEQ ID NO: 14 - protein, 13 aa, Homo sapiens
ASSQVYGGDTQYF

SEQ ID NO: 15 - protein, 14 aa, Homo sapiens
ASSWTGGHTDTQYF

SEQ ID NO: 16 - protein, 12 aa, Homo sapiens
ASSEQREGEQYF

SEQ ID NO: 17 - protein, 14 aa, Homo sapiens
ASSIRAGGTDTQYF

SEQ ID NO: 18 - protein, 12 aa, Homo sapiens
ASSIRDTDTQYF

SEQ ID NO: 19 - protein, 13 aa, Homo sapiens
ASSIRTGDHEQYF

SEQ ID NO: 20 - protein, 13 aa, Homo sapiens
ASSIRTGGETQYF

SEQ ID NO: 21 - protein, 12 aa, Homo sapiens
ASSIRTTDTQYF

SEQ ID NO: 22 - protein, 12 aa, Homo sapiens
ASSIRYTDTQYF

SEQ ID NO: 23 - protein, 13 aa, Homo sapiens
ASSLASYGDTQYF

SEQ ID NO: 24 - protein, 12 aa, Homo sapiens
ASSLRFTDTQYF

SEQ ID NO: 25 - protein, 14 aa, Homo sapiens
ASSLVAYSGETQYF

SEQ ID NO: 26 - protein, 12 aa, Homo sapiens
ASSTRTTDTQYF

SEQ ID NO: 27 - protein, 12 aa, Homo sapiens
ASSTRWSDTQYF

SEQ ID NO: 28 - protein, 12 aa, Homo sapiens
ASSVRFTDTQYF

SEQ ID NO: 29 - protein, 12 aa, Homo sapiens
ATSIRFTDTQYF

SEQ ID NO: 30 - protein, 12 aa, Homo sapiens
ASSVRFTDTQYF

SEQ ID NO: 31 - protein, 14 aa, Homo sapiens
ASSIRQGGNHEQYF

SEQ ID NO: 32 - protein, 12 aa, Homo sapiens
ASSFRSTDTQYF

SEQ ID NO: 33 - protein, 14 aa, Homo sapiens
ASSQDGQGPETQYF

SEQ ID NO: 34 - protein, 12 aa, Homo sapiens
ASSVRSTDTQYF

SEQ ID NO: 35 - protein, 12 aa, Homo sapiens
ASSFRVSDTQYF

SEQ ID NO: 36 - protein, 12 aa, Homo sapiens
ASSIRNTDTQYF

SEQ ID NO: 37 - protein, 14 aa, Homo sapiens
ASSLRAGGVDTQYF

SEQ ID NO: 38 - protein, 13 aa, Homo sapiens
ASSQPHRGDEQFF

SEQ ID NO: 39 - protein, 12 aa, Homo sapiens
ASSARFTDTQYF

SEQ ID NO: 40 - protein, 12 aa, Homo sapiens
ASSFRTSDTQYF

SEQ ID NO: 41 - protein, 12 aa, Homo sapiens
ASSIRSTDTQYF

SEQ ID NO: 42 - protein, 12 aa, Homo sapiens
ASSLRSGDTQYF

SEQ ID NO: 43 - protein, 12 aa, Homo sapiens
ASSLVAWETQYF

SEQ ID NO: 44 - protein, 14 aa, Homo sapiens
ASSVRGGEADTQYF

SEQ ID NO: 45 - protein, 13 aa, Homo sapiens
  misc_feature (2)..(9): Xaa can be any naturally occurring amino acid
CXXXXXXXXYWGI

SEQ ID NO: 46 - protein, 12 aa, Homo sapiens
CASSPGTDTQYF

SEQ ID NO: 47 - protein, 16 aa, Homo sapiens
CASSLPPRGGGYEQYF

SEQ ID NO: 48 - protein, 11 aa, Homo sapiens
CASSFGGELFF

SEQ ID NO: 49 - protein, 15 aa, Homo sapiens
CASSPEDPYTDTQYF

SEQ ID NO: 50 - protein, 12 aa, Homo sapiens
CASAGNYEKLFF

SEQ ID NO: 51 - protein, 15 aa, Homo sapiens
CASSPEREVYNEQFF

SEQ ID NO: 52 - protein, 12 aa, Homo sapiens
CASSAGHPEQFF

SEQ ID NO: 53 - protein, 15 aa, Homo sapiens
CASSPFSGDYYEQYF

SEQ ID NO: 54 - protein, 12 aa, Homo sapiens
CASSLINTEAFF

SEQ ID NO: 55 - protein, 12 aa, Homo sapiens
CASSPGTNIQYF

SEQ ID NO: 56 - protein, 17 aa, Homo sapiens
CASSNLRQGAAGNTIYF

SEQ ID NO: 57 - protein, 14 aa, Homo sapiens
CASSPGTVVYGYTF

SEQ ID NO: 58 - protein, 16 aa, Homo sapiens
CASSQEEQGAFYEQFF

SEQ ID NO: 59 - protein, 12 aa, Homo sapiens
CASSPGTYEQYF

SEQ ID NO: 60 - protein, 12 aa, Homo sapiens
CASTEGQAEAFF

SEQ ID NO: 61 - protein, 12 aa, Homo sapiens
CASSPGVYEQYF

SEQ ID NO: 62 - protein, 14 aa, Homo sapiens
CATSGTSGYNEQFF

SEQ ID NO: 63 - protein, 13 aa, Homo sapiens
CASSPSNTGELFF

SEQ ID NO: 64 - protein, 14 aa, Homo sapiens
CSVDGNYLTDTQYF

SEQ ID NO: 65 - protein, 16 aa, Homo sapiens
CASSQAGALWDYGYTF

SEQ ID NO: 66 - protein, 14 aa, Homo sapiens
CASRIQGEGSPLHF

SEQ ID NO: 67 - protein, 15 aa, Homo sapiens
CASSQGIRSEYEQYF

SEQ ID NO: 68 - protein, 12 aa, Homo sapiens
CASSPGTNTQYF

SEQ ID NO: 69 - protein, 14 aa, Homo sapiens
CASSQLPVNSPLHF

SEQ ID NO: 70 - protein, 14 aa, Homo sapiens
CASSQDLGDYGYTF

SEQ ID NO: 71 - protein, 14 aa, Homo sapiens
CASSSGLATDTQYF

SEQ ID NO: 72 - protein, 11 aa, Homo sapiens
CSVEMNTEAFF

SEQ ID NO: 73 - protein, 14 aa, Homo sapiens
CASSTGHMEDTQYF

SEQ ID NO: 74 - protein, 14 aa, Homo sapiens
CAISDPPLATEAFF

SEQ ID NO: 75 - protein, 15 aa, Homo sapiens
CASSTGVSGANVLTF

SEQ ID NO: 76 - protein, 13 aa, Homo sapiens
CAISSGQVPEQFF

SEQ ID NO: 77 - protein, 15 aa, Homo sapiens
CASSVEGGMGEKLFF

SEQ ID NO: 78 - protein, 14 aa, Homo sapiens
CASEMDANTGELFF

SEQ ID NO: 79 - protein, 16 aa, Homo sapiens
CASSVGAGVNSYEQYF

SEQ ID NO: 80 - protein, 13 aa, Homo sapiens
CASKLGGATEAFF

SEQ ID NO: 81 - protein, 16 aa, Homo sapiens
CASSVRAGTGTYEQYF

SEQ ID NO: 82 - protein, 14 aa, Homo sapiens
CASNQGQGVETQYF

SEQ ID NO: 83 - protein, 14 aa, Homo sapiens
CASSVSTGSYEQYF

SEQ ID NO: 84 - protein, 12 aa, Homo sapiens
CASSEGTYEQYF

SEQ ID NO: 85 - protein, 15 aa, Homo sapiens
CASSWDRATNEKLFF

SEQ ID NO: 86 - protein, 12 aa, Homo sapiens
CASSFGTDTQYF

SEQ ID NO: 87 - protein, 15 aa, Homo sapiens
CASSYAPTGNYGYTF

SEQ ID NO: 88 - protein, 12 aa, Homo sapiens
CASSFGTSDQFF

SEQ ID NO: 89 - protein, 14 aa, Homo sapiens
CASSYKRGPGELFF

SEQ ID NO: 90 - protein, 13 aa, Homo sapiens
CASSFLGTYEQYF

SEQ ID NO: 91 - protein, 18 aa, Homo sapiens
CASSYSMASGGAQETQYF

SEQ ID NO: 92 - protein, 14 aa, Homo sapiens
CASSFPNPTFEAFF

SEQ ID NO: 93 - protein, 15 aa, Homo sapiens
CASSYTAGSNQPQHF

SEQ ID NO: 94 - protein, 14 aa, Homo sapiens
CASSFRGQGNEQYF

SEQ ID NO: 95 - protein, 14 aa, Homo sapiens
CASSYWEEGGGAFF

SEQ ID NO: 96 - protein, 14 aa, Homo sapiens
CASSFTGSSYEQYF

SEQ ID NO: 97 - protein, 15 aa, Homo sapiens
CASSYWGPMNTEAFF

SEQ ID NO: 98 - protein, 13 aa, Homo sapiens
CASSGGTVYGYTF

SEQ ID NO: 99 - protein, 16 aa, Homo sapiens
CAWSVKTLRRADTQYF

SEQ ID NO: 100 - protein, 15 aa, Homo sapiens
CASSGSGGVTGELFF

SEQ ID NO: 101 - protein, 12 aa, Homo sapiens
CSAAGHFYEQYF

SEQ ID NO: 102 - protein, 15 aa, Homo sapiens
CASSLASVGSTEAFF

SEQ ID NO: 103 - protein, 12 aa, Homo sapiens
CSAMTQEDYAFF

SEQ ID NO: 104 - protein, 15 aa, Homo sapiens
CASSLGTGGYNEQFF

SEQ ID NO: 105 - protein, 15 aa, Homo sapiens
CSARDFSLRTGELFF

SEQ ID NO: 106 - protein, 14 aa, Homo sapiens
CASSLLGLTGELFF

SEQ ID NO: 107 - protein, 18 aa, Homo sapiens
CSARDMFGGHVSGNTIYF

SEQ ID NO: 108 - protein, 15 aa, Homo sapiens
CASSLNLGQNTEAFF

SEQ ID NO: 109 - protein, 15 aa, Homo sapiens
CSVDGLAGITDTQYF

SEQ ID NO: 110 - protein, 16 aa, Homo sapiens
CASSLPLGQGNQPQHF

SEQ ID NO: 111 - protein, 17 aa, Homo sapiens
CASSLTRQGGEGSPLHF

SEQ ID NO: 112 - protein, 15 aa, Homo sapiens
CASSLVADSYNEQFF

SEQ ID NO: 113 - protein, 14 aa, Homo sapiens
CASSLLAGGAEQFF

SEQ ID NO: 114 - protein, 14 aa, Homo sapiens
CASSLVGPGDTQYF

SEQ ID NO: 115 - protein, 12 aa, Homo sapiens
CASSPGTALAFF

SEQ ID NO: 116 - protein, 15 aa, Homo sapiens
CASSSSDRATDTQYF

SEQ ID NO: 117 - protein, 13 aa, Homo sapiens
CASSQDGGARQHF

SEQ ID NO: 118 - protein, 17 aa, Homo sapiens
CASSQDSGGARNNEQFF

SEQ ID NO: 119 - protein, 17 aa, Homo sapiens
CASSQDWALGWGYGYTF

SEQ ID NO: 120 - protein, 14 aa, Homo sapiens
CASSQLTQNTEAFF

SEQ ID NO: 121 - protein, 14 aa, Homo sapiens
CASSYRQAGYEQYF

SEQ ID NO: 122 - protein, 16 aa, Homo sapiens
CASSQPFVGSGNTIYF

SEQ ID NO: 123 - protein, 13 aa, Homo sapiens
CASSRDWGETQYF

SEQ ID NO: 124 - protein, 14 aa, Homo sapiens
CASSLPTAVTEAFF

SEQ ID NO: 125 - protein, 17 aa, Homo sapiens
CASSRGLAGESTDTQYF

SEQ ID NO: 126 - protein, 13 aa, Homo sapiens
CASSRPASYEQYF

SEQ ID NO: 127 - protein, 14 aa, Homo sapiens
CASSSGADLYGYTF

SEQ ID NO: 128 - protein, 15 aa, Homo sapiens
CASSDRGFPSYEQYF

SEQ ID NO: 129 - protein, 14 aa, Homo sapiens
CASSSGLSYNEQFF

SEQ ID NO: 130 - protein, 13 aa, Homo sapiens
CASSSGTLETQYF

SEQ ID NO: 131 - protein, 12 aa, Homo sapiens
CASSFGNQPQHF

SEQ ID NO: 132 - protein, 15 aa, Homo sapiens
CASSSSSGSTYEQYF

SEQ ID NO: 133 - protein, 17 aa, Homo sapiens
CASSFSLAGWSYNEQFF

SEQ ID NO: 134 - protein, 15 aa, Homo sapiens
CASSSWTGTNTEAFF

SEQ ID NO: 135 - protein, 16 aa, Homo sapiens
CASSFSNGENTDTQYF

SEQ ID NO: 136 - protein, 16 aa, Homo sapiens
CASSWLAGGPAGELFF

SEQ ID NO: 137 - protein, 14 aa, Homo sapiens
CASSHLGGGNTIYF

SEQ ID NO: 138 - protein, 18 aa, Homo sapiens
CASSYATGTPSSYNEQFF

SEQ ID NO: 139 - protein, 15 aa, Homo sapiens
CASSLAGDSYNEQFF

SEQ ID NO: 140 - protein, 15 aa, Homo sapiens
CASSYGSDSYNEQFF

SEQ ID NO: 141 - protein, 15 aa, Homo sapiens
CASSLGQGTPDTQYF

SEQ ID NO: 142 - protein, 14 aa, Homo sapiens
CSARVSGSLYEQYF

SEQ ID NO: 143 - protein, 16 aa, Homo sapiens
CASSLSLAGDTGELFF

SEQ ID NO: 144 - protein, 14 aa, Homo sapiens
CSVEMSGGDYEQYF

SEQ ID NO: 145 - protein, 16 aa, Homo sapiens
CASSYDVRSGNYEQYF

SEQ ID NO: 146 - protein, 15 aa, Homo sapiens
CASSLSADRDGGYTF

SEQ ID NO: 147 - protein, 14 aa, Homo sapiens
CASSPSDPSDTQYF

SEQ ID NO: 148 - protein, 15 aa, Homo sapiens
CASSLSQGGHNEQFF

SEQ ID NO: 149 - protein, 15 aa, Homo sapiens
CASSPGTGSGDEQFF

SEQ ID NO: 150 - protein, 17 aa, Homo sapiens
CASSLVPPGGFSYEQYF

SEQ ID NO: 151 - protein, 13 aa, Homo sapiens
CASSVGGVQPQHF

SEQ ID NO: 152 - protein, 16 aa, Homo sapiens
CASSMGQGNSGETQYF

SEQ ID NO: 153 - protein, 14 aa, Homo sapiens
CASNLAGGSNEQFF

SEQ ID NO: 154 - protein, 12 aa, Homo sapiens
CASSPGGAGYTF

SEQ ID NO: 155 - protein, 14 aa, Homo sapiens
CASSFGQVTYEQYF

SEQ ID NO: 156 - protein, 14 aa, Homo sapiens
CASSPGGWSYEQYF

SEQ ID NO: 157 - protein, 13 aa, Homo sapiens
CASSLGGAETQYF

SEQ ID NO: 158 - protein, 15 aa, Homo sapiens
CASSPGQGNNSPLHF

SEQ ID NO: 159 - protein, 14 aa, Homo sapiens
CASSQDWGDYGYTF

SEQ ID NO: 160 - protein, 17 aa, Homo sapiens
CASSPISRDRNTGELFF

SEQ ID NO: 161 - protein, 17 aa, Homo sapiens
CASSSEQDRGSENTIYF

SEQ ID NO: 162 - protein, 14 aa, Homo sapiens
CASSPLGSGTEAFF

SEQ ID NO: 163 - protein, 12 aa, Homo sapiens
CASSWSGYEQYF

SEQ ID NO: 164 - protein, 14 aa, Homo sapiens
CASSPNPNTGELFF

SEQ ID NO: 165 - protein, 12 aa, Homo sapiens
CASSWTGNEQFF

SEQ ID NO: 166 - protein, 16 aa, Homo sapiens
CASSPPDRGYDNEQFF

SEQ ID NO: 167 - protein, 18 aa, Homo sapiens
CAIRATGLAGVDTGELFF

SEQ ID NO: 168 - protein, 12 aa, Homo sapiens
CASSPPLTEAFF

SEQ ID NO: 169 - protein, 14 aa, Homo sapiens
CAISEQEYGTEAFF

SEQ ID NO: 170 - protein, 16 aa, Homo sapiens
CASSPRLAGAKDTQYF

SEQ ID NO: 171 - protein, 16 aa, Homo sapiens
CAISFGTGEAPRGYTF

SEQ ID NO: 172 - protein, 16 aa, Homo sapiens
CASSPRSAGGPYEQYF

SEQ ID NO: 173 - protein, 14 aa, Homo sapiens
CAISPDRVTFEVFF

SEQ ID NO: 174 - protein, 17 aa, Homo sapiens
CASSPTSGRTTSYEQYF

SEQ ID NO: 175 - protein, 12 aa, Homo sapiens
CALWGGAYEQYF

SEQ ID NO: 176 - protein, 15 aa, Homo sapiens
CASSQDGGTYNEQFF

SEQ ID NO: 177 - protein, 15 aa, Homo sapiens
CASCFTSLGTGELFF

SEQ ID NO: 178 - protein, 14 aa, Homo sapiens
CASSQGDRDYEQFF

SEQ ID NO: 179 - protein, 10 aa, Homo sapiens
CASGSSEQFF

SEQ ID NO: 180 - protein, 15 aa, Homo sapiens
CASSSGTAQSEKLFF

SEQ ID NO: 181 - protein, 13 aa, Homo sapiens
CASGTSQAYEQYF

SEQ ID NO: 182 - protein, 15 aa, Homo sapiens
CASSSLGTEVYEQYF

SEQ ID NO: 183 - protein, 11 aa, Homo sapiens
CASIQGHEQYF

SEQ ID NO: 184 - protein, 14 aa, Homo sapiens
CASSSPGDSYEQYF

SEQ ID NO: 185 - protein, 15 aa, Homo sapiens
CASNAGAGFGYEQYF

SEQ ID NO: 186 - protein, 16 aa, Homo sapiens
CASSSSGRAQTDTQYF

SEQ ID NO: 187 - protein, 15 aa, Homo sapiens
CASRGTVRGGYEQYF

SEQ ID NO: 188 - protein, 17 aa, Homo sapiens
CASSSTGGISWNTEAFF

SEQ ID NO: 189 - protein, 12 aa, Homo sapiens
CASRLGTAPAFF

SEQ ID NO: 190 - protein, 16 aa, Homo sapiens
CASSSTGPPFNYGYTF

SEQ ID NO: 191 - protein, 15 aa, Homo sapiens
CASRRTAATNEKLFF

SEQ ID NO: 192 - protein, 16 aa, Homo sapiens
CASSSTPGGLWYGYTF

SEQ ID NO: 193 - protein, 14 aa, Homo sapiens
CASSARNSNQPQHF

SEQ ID NO: 194 - protein, 11 aa, Homo sapiens
CASSSTYEQYF

SEQ ID NO: 195 - protein, 14 aa, Homo sapiens
CASSARTGAYGYTF

SEQ ID NO: 196 - protein, 18 aa, Homo sapiens
CASSTRTTHTYSNQPQHF

SEQ ID NO: 197 - protein, 13 aa, Homo sapiens
CASSDEAGYGYTF

SEQ ID NO: 198 - protein, 14 aa, Homo sapiens
CASSVKRLNTEAFF

SEQ ID NO: 199 - protein, 17 aa, Homo sapiens
CASSDLPSGAPQETQYF

SEQ ID NO: 200 - protein, 13 aa, Homo sapiens
CASSYGQSFEQYF

SEQ ID NO: 201 - protein, 15 aa, Homo sapiens
CASSETASSTDTQYF

SEQ ID NO: 202 - protein, 14 aa, Homo sapiens
CASSYKQGIHEQYF

SEQ ID NO: 203 - protein, 11 aa, Homo sapiens
CASSFAETQYF

SEQ ID NO: 204 - protein, 14 aa, Homo sapiens
CASSYSQGNYGYTF

SEQ ID NO: 205 - protein, 14 aa, Homo sapiens
CASSFDPRGEKLFF

SEQ ID NO: 206 - protein, 16 aa, Homo sapiens
CASSYTPGGNTDTQYF

SEQ ID NO: 207 - protein, 15 aa, Homo sapiens
CASSFGTGSSETQYF

SEQ ID NO: 208 - protein, 14 aa, Homo sapiens
CASSYWAGDYGYTF

SEQ ID NO: 209 - protein, 13 aa, Homo sapiens
CASSFGVYNEQFF

SEQ ID NO: 210 - protein, 13 aa, Homo sapiens
CASTAGFNQPQHF

SEQ ID NO: 211 - protein, 16 aa, Homo sapiens
CASSFPPSGDTDTQYF

SEQ ID NO: 212 - protein, 15 aa, Homo sapiens
CASTDVTSGQETQYF

SEQ ID NO: 213 - protein, 15 aa, Homo sapiens
CASSFSGSDTGELFF

SEQ ID NO: 214 - protein, 15 aa, Homo sapiens
CASTGIAGPTDTQYF

SEQ ID NO: 215 - protein, 15 aa, Homo sapiens
CASSFTGTPTYEQYF

SEQ ID NO: 216 - protein, 13 aa, Homo sapiens
CATIGPAGDTQYF

SEQ ID NO: 217 - protein, 14 aa, Homo sapiens
CASSHGQGNQPQHF

SEQ ID NO: 218 - protein, 11 aa, Homo sapiens
CATPSGNTIYF

SEQ ID NO: 219 - protein, 15 aa, Homo sapiens
CASSHPTRSYNEQFF

SEQ ID NO: 220 - protein, 15 aa, Homo sapiens
CATSDLGLGVNEQFF

SEQ ID NO: 221 - protein, 20 aa, Homo sapiens
CASSHRRGIPPPPLYNEQFF

SEQ ID NO: 222 - protein, 13 aa, Homo sapiens
CATSRAQGQPQHF

SEQ ID NO: 223 - protein, 14 aa, Homo sapiens
CASSIAARAGELFF

SEQ ID NO: 224 - protein, 19 aa, Homo sapiens
CATSRDHSSGASQGNIQYF

SEQ ID NO: 225 - protein, 14 aa, Homo sapiens
CASSIGVLNTEAFF

SEQ ID NO: 226 - protein, 16 aa, Homo sapiens
CATSRDNTGYTGELFF

SEQ ID NO: 227 - protein, 12 aa, Homo sapiens
CASSKLDSGYTF

SEQ ID NO: 228 - protein, 14 aa, Homo sapiens
CATSRDQGSDTQYF

SEQ ID NO: 229 - protein, 12 aa, Homo sapiens
CASSKPPETQYF

SEQ ID NO: 230 - protein, 15 aa, Homo sapiens
CAWDSTGISYNEQFF

SEQ ID NO: 231 - protein, 16 aa, Homo sapiens
CASSLAGGIAKNIQYF

SEQ ID NO: 232 - protein, 14 aa, Homo sapiens
CAWRATEGQETQYF

SEQ ID NO: 233 - protein, 15 aa, Homo sapiens
CASSLAPWMDYGYTF

SEQ ID NO: 234 - protein, 14 aa, Homo sapiens
CAWSDSGSSYEQYF

SEQ ID NO: 235 - protein, 15 aa, Homo sapiens
CASSLFTGGTYEQYF

SEQ ID NO: 236 - protein, 15 aa, Homo sapiens
CAWSVTGTRLYEQYF

SEQ ID NO: 237 - protein, 16 aa, Homo sapiens
CASSLGGLELGFEQFF

SEQ ID NO: 238 - protein, 14 aa, Homo sapiens
CSALDSYSNQPQHF

SEQ ID NO: 239 - protein, 14 aa, Homo sapiens
CASSLGGSNQPQHF

SEQ ID NO: 240 - protein, 16 aa, Homo sapiens
CSARAAIGTMNTEAFF

SEQ ID NO: 241 - protein, 14 aa, Homo sapiens
CASSLGLGTGELFF

SEQ ID NO: 242 - protein, 16 aa, Homo sapiens
CSARDADGYESEKLFF

SEQ ID NO: 243 - protein, 12 aa, Homo sapiens
CASSLGRVEAFF

SEQ ID NO: 244 - protein, 16 aa, Homo sapiens
CSARDRQGYSNQPQHF

SEQ ID NO: 245 - protein, 14 aa, Homo sapiens
CASSLGTSQETQYF

SEQ ID NO: 246 - protein, 15 aa, Homo sapiens
CSARPPNGRYNEQFF

SEQ ID NO: 247 - protein, 19 aa, Homo sapiens
CASSLLRLAGETTYNEQFF

SEQ ID NO: 248 - protein, 18 aa, Homo sapiens
CSARVKGLAGIRSYEQYF

SEQ ID NO: 249 - protein, 15 aa, Homo sapiens
CASSLPGTGTSPLHF

SEQ ID NO: 250 - protein, 14 aa, Homo sapiens
CSDTVRRGPGGYTF

SEQ ID NO: 251 - protein, 14 aa, Homo sapiens
CASSLRGYTDTQYF

SEQ ID NO: 252 - protein, 14 aa, Homo sapiens
CSVLGQGPSYEQYF

SEQ ID NO: 253 - protein, 14 aa, Homo sapiens
CASSLRQGGYEQYF

SEQ ID NO: 254 - protein, 11 aa, Homo sapiens
CSVVEGGEQYF

SEQ ID NO: 255 - protein, 13 aa, Homo sapiens
CAWSGVSADTQYF

SEQ ID NO: 256 - protein, 13 aa, Homo sapiens
CASSLSGWTEAFF

SEQ ID NO: 257 - protein, 14 aa, Homo sapiens
CASSLGARTGELFF

SEQ ID NO: 258 - protein, 14 aa, Homo sapiens
CASSPRSRTDTQYF

SEQ ID NO: 259 - protein, 14 aa, Homo sapiens
CASSLTASNQPQHF

SEQ ID NO: 260 - protein, 13 aa, Homo sapiens
CASSPAGYNEQFF

SEQ ID NO: 261 - protein, 11 aa, Homo sapiens
CSAREGDTQYF

SEQ ID NO: 262 - protein, 14 aa, Homo sapiens
CASSPGLVVDEQYF

SEQ ID NO: 263 - protein, 10 aa, Homo sapiens
CASSSGELFF

SEQ ID NO: 264 - protein, 16 aa, Homo sapiens
CASSPPGQGITDTQYF

SEQ ID NO: 265 - protein, 11 aa, Homo sapiens
CASDRGETQYF

SEQ ID NO: 266 - protein, 17 aa, Homo sapiens
CASSPRNKRKADQPQHF

SEQ ID NO: 267 - protein, 11 aa, Homo sapiens
CASGESNEQYF

SEQ ID NO: 268 - protein, 16 aa, Homo sapiens
CASSPRVDRGLHEQYF

SEQ ID NO: 269 - protein, 14 aa, Homo sapiens
CASKPLQGYNEQFF

SEQ ID NO: 270 - protein, 14 aa, Homo sapiens
CASNIRASNQPQHF

SEQ ID NO: 271 - protein, 15 aa, Homo sapiens
CASSPWDGSSYEQYF

SEQ ID NO: 272 - protein, 14 aa, Homo sapiens
CASSQDGGDYGYTF

SEQ ID NO: 273 - protein, 12 aa, Homo sapiens
CASQRQSYEQYF

SEQ ID NO: 274 - protein, 17 aa, Homo sapiens
CASRFGQGGNSNQPQHF

SEQ ID NO: 275 - protein, 15 aa, Homo sapiens
CASSQGRGKVYEQYF

SEQ ID NO: 276 - protein, 14 aa, Homo sapiens
CASRSRGTIYEQYF

SEQ ID NO: 277 - protein, 15 aa, Homo sapiens
CASSQTGLTNYGYTF

SEQ ID NO: 278 - protein, 13 aa, Homo sapiens
CASSDGLAYEQYF

SEQ ID NO: 279 - protein, 11 aa, Homo sapiens
CASSRGVEQYF

SEQ ID NO: 280 - protein, 15 aa, Homo sapiens
CASSDTGSINQPQHF

SEQ ID NO: 281 - protein, 13 aa, Homo sapiens
CASSRSGNTEAFF

SEQ ID NO: 282 - protein, 15 aa, Homo sapiens
CASSFASSSGNTIYF

SEQ ID NO: 283 - protein, 14 aa, Homo sapiens
CASSSPLGGYGYTF

SEQ ID NO: 284 - protein, 17 aa, Homo sapiens
CASSFIVLSGSSYEQYF

SEQ ID NO: 285 - protein, 16 aa, Homo sapiens
CASSSPVRSGANVLTF

SEQ ID NO: 286 - protein, 14 aa, Homo sapiens
CASSFSGRTYEQYF

SEQ ID NO: 287 - protein, 13 aa, Homo sapiens
CASSFSQVDEQFF

SEQ ID NO: 288 - protein, 15 aa, Homo sapiens
CASSWMTRIYNEQFF

SEQ ID NO: 289 - protein, 15 aa, Homo sapiens
CASSGQQGGSYGYTF

SEQ ID NO: 290 - protein, 13 aa, Homo sapiens
CASSYDSAYEQYF

SEQ ID NO: 291 - protein, 13 aa, Homo sapiens
CASSGSATGELFF

SEQ ID NO: 292 - protein, 12 aa, Homo sapiens
CASSYSVAGAFF

SEQ ID NO: 293 - protein, 11 aa, Homo sapiens
CASSLDDGYTF

SEQ ID NO: 294 - protein, 13 aa, Homo sapiens
CASSLDRGDTQYF

SEQ ID NO: 295 - protein, 15 aa, Homo sapiens
CASTFSYAGTDTQYF

SEQ ID NO: 296 - protein, 16 aa, Homo sapiens
CASSLEQSAMNTEAFF

SEQ ID NO: 297 - protein, 16 aa, Homo sapiens
CATSTLQGGPRDEQFF

SEQ ID NO: 298 - protein, 11 aa, Homo sapiens
CASSLGGELFF

SEQ ID NO: 299 - protein, 13 aa, Homo sapiens
CATSVTGSYGYTF

SEQ ID NO: 300 - protein, 15 aa, Homo sapiens
CASSLGGSGSYEQYF

SEQ ID NO: 301 - protein, 12 aa, Homo sapiens
CATTGSSYEQYF

SEQ ID NO: 302 - protein, 12 aa, Homo sapiens
CASSLGNTEAFF

SEQ ID NO: 303 - protein, 13 aa, Homo sapiens
CAWNRGGSSPLHF

SEQ ID NO: 304 - protein, 14 aa, Homo sapiens
CASSLGQGAQTQYF

SEQ ID NO: 305 - protein, 13 aa, Homo sapiens
CAWRRQGEEKLFF

SEQ ID NO: 306 - protein, 13 aa, Homo sapiens
CAYSPGKNTEAFF

SEQ ID NO: 307 - protein, 15 aa, Homo sapiens
CASSLLGGVQETQYF

SEQ ID NO: 308 - protein, 12 aa, Homo sapiens
CSAPGQRNTIYF

SEQ ID NO: 309 - protein, 17 aa, Homo sapiens
CASSLRGTSSYNSPLHF

SEQ ID NO: 310 - protein, 15 aa, Homo sapiens
CSARDPDSPPGGYTF

SEQ ID NO: 311 - protein, 15 aa, Homo sapiens
CASSLSGGSWTEAFF

SEQ ID NO: 312 - protein, 11 aa, Homo sapiens
CSVGGGEDYTF

SEQ ID NO: 313 - protein, 17 aa, Homo sapiens
CASSQERGGKWAYEQYF

SEQ ID NO: 314 - protein, 16 aa, Homo sapiens
CASSLPVAGGQETQYF

SEQ ID NO: 315 - protein, 13 aa, Homo sapiens
CATSDLLTGNAFF

SEQ ID NO: 316 - protein, 14 aa, Homo sapiens
CASSLTGGSYGYTF

SEQ ID NO: 317 - protein, 14 aa, Homo sapiens
CASSLNGGSYEQYF

SEQ ID NO: 318 - protein, 14 aa, Homo sapiens
CASSLVWNTDTQYF

SEQ ID NO: 319 - protein, 20 aa, Homo sapiens
CASRPIPGQEKSSGANVLTF

SEQ ID NO: 320 - protein, 14 aa, Homo sapiens
CASSPDGTGIEQFF

SEQ ID NO: 321 - protein, 16 aa, Homo sapiens
CASSFFPRTGSNEQFF

SEQ ID NO: 322 - protein, 15 aa, Homo sapiens
CASSPGAFTNTEAFF

SEQ ID NO: 323 - protein, 16 aa, Homo sapiens
CASSQEGVGGNYGYTF

SEQ ID NO: 324 - protein, 13 aa, Homo sapiens
CASSPWGSYEQYF

SEQ ID NO: 325 - protein, 15 aa, Homo sapiens
CASSQEQGTNYGYTF

SEQ ID NO: 326 - protein, 17 aa, Homo sapiens
CASSPYIGEVGNTEAFF

SEQ ID NO: 327 - protein, 17 aa, Homo sapiens
CASSQLTLGPAKNIQYF

SEQ ID NO: 328 - protein, 14 aa, Homo sapiens
CASSQEESADTQYF

SEQ ID NO: 329 - protein, 15 aa, Homo sapiens
CASSQQLNYNSPLHF

SEQ ID NO: 330 - protein, 18 aa, Homo sapiens
CASSQELGQSSYNSPLHF

SEQ ID NO: 331 - protein, 18 aa, Homo sapiens
CASSVEGGGGPSTDTQYF

SEQ ID NO: 332 - protein, 15 aa, Homo sapiens
CASSQERGTAYGYTF

SEQ ID NO: 333 - protein, 15 aa, Homo sapiens
CASSVPKGGFNEQFF

SEQ ID NO: 334 - protein, 14 aa, Homo sapiens
CASSQERSADTQYF

SEQ ID NO: 335 - protein, 12 aa, Homo sapiens
CASGSGVTEAFF

SEQ ID NO: 336 - protein, 16 aa, Homo sapiens
CASSQPQGWGNTEAFF

SEQ ID NO: 337 - protein, 14 aa, Homo sapiens
CASRPGATNYGYTF

SEQ ID NO: 338 - protein, 12 aa, Homo sapiens
CASSSYTGELFF

SEQ ID NO: 339 - protein, 14 aa, Homo sapiens
CASSLDGGVNGYTF

SEQ ID NO: 340 - protein, 14 aa, Homo sapiens
CASSYIQGNQPQHF

SEQ ID NO: 341 - protein, 15 aa, Homo sapiens
CASSLDGVQIYGYTF

SEQ ID NO: 342 - protein, 15 aa, Homo sapiens
CASSYQGGGTDTQYF

SEQ ID NO: 343 - protein, 18 aa, Homo sapiens
CASTRTGTGPATNEKLFF

SEQ ID NO: 344 - protein, 15 aa, Homo sapiens
CASSLGQGVGTEAFF

SEQ ID NO: 345 - protein, 13 aa, Homo sapiens
CSASLQENTEAFF

SEQ ID NO: 346 - protein, 11 aa, Homo sapiens
CASSLGYEQYF

SEQ ID NO: 347 - protein, 15 aa, Homo sapiens
CSVQERDSANYGYTF

SEQ ID NO: 348 - protein, 12 aa, Homo sapiens
CASRTGNQPQHF

SEQ ID NO: 349 - protein, 12 aa, Homo sapiens
CASSLGSNEQFF

SEQ ID NO: 350 - protein, 12 aa, Homo sapiens
CASRGGNTEAFF

SEQ ID NO: 351 - protein, 13 aa, Homo sapiens
CASSLGSRETQYF

SEQ ID NO: 352 - protein, 14 aa, Homo sapiens
CASSFRVGYNEQFF

SEQ ID NO: 353 - protein, 12 aa, Homo sapiens
CASSLGTSEQYF

SEQ ID NO: 354 - protein, 12 aa, Homo sapiens
CASIAGNTEAFF

SEQ ID NO: 355 - protein, 14 aa, Homo sapiens
CASSLGTTSNEQYF

SEQ ID NO: 356 - protein, 11 aa, Homo sapiens
CASSSSYEQYF

SEQ ID NO: 357 - protein, 12 aa, Homo sapiens
CASSLLNTEAFF

SEQ ID NO: 358 - protein, 13 aa, Homo sapiens
CASSLGNRPEAFF

SEQ ID NO: 359 - protein, 15 aa, Homo sapiens
CASSLSRDNYNEQFF

SEQ ID NO: 360 - protein, 12 aa, Homo sapiens
CASSFGSDTQYF

SEQ ID NO: 361 - protein, 14 aa, Homo sapiens
CASSLVGVADTQYF

SEQ ID NO: 362 - protein, 13 aa, Homo sapiens
CAWRSGGASPLHF

SEQ ID NO: 363 - protein, 15 aa, Homo sapiens
CASSPEPTGSNEQFF

SEQ ID NO: 364 - protein, 11 aa, Homo sapiens
CASKAGEFEFF

SEQ ID NO: 365 - protein, 14 aa, Homo sapiens
CASSPGQGVTEAFF

SEQ ID NO: 366 - protein, 11 aa, Homo sapiens
CASSLEYEQYF

SEQ ID NO: 367 - protein, 13 aa, Homo sapiens
CASSPGTVYGYTF

SEQ ID NO: 368 - protein, 16 aa, Homo sapiens
CASSRLGGRAGETQYF

SEQ ID NO: 369 - protein, 16 aa, Homo sapiens
CASSPQGVGADYGYTF

SEQ ID NO: 370 - protein, 13 aa, Homo sapiens
CATTGGLGTEAFF

SEQ ID NO: 371 - protein, 17 aa, Homo sapiens
CASSPRAEDWTYYGYTF

SEQ ID NO: 372 - protein, 12 aa, Homo sapiens
CAGARGNQPQHF

SEQ ID NO: 373 - protein, 16 aa, Homo sapiens
CASSQDGISGSGEQYF

SEQ ID NO: 374 - protein, 12 aa, Homo sapiens
CAGTSGNQPQHF

SEQ ID NO: 375 - protein, 15 aa, Homo sapiens
CASSQGATSNQPQHF

SEQ ID NO: 376 - protein, 14 aa, Homo sapiens
CASEPLAGTNEQFF

SEQ ID NO: 377 - protein, 16 aa, Homo sapiens
CASSRTSGGTGETQYF

SEQ ID NO: 378 - protein, 15 aa, Homo sapiens
CASGQDWSSYNEQFF

SEQ ID NO: 379 - protein, 16 aa, Homo sapiens
CASSSDSHYSNQPQHF

SEQ ID NO: 380 - protein, 14 aa, Homo sapiens
CASMTGNSNQPQHF

SEQ ID NO: 381 - protein, 16 aa, Homo sapiens
CASSSLGGRAGETQYF

SEQ ID NO: 382 - protein, 13 aa, Homo sapiens
CASRPANQETQYF

SEQ ID NO: 383 - protein, 14 aa, Homo sapiens
CASSTERLGDGYTF

SEQ ID NO: 384 - protein, 13 aa, Homo sapiens
CASRSMDTYEQYF

SEQ ID NO: 385 - protein, 13 aa, Homo sapiens
CASSTGNQETQYF

SEQ ID NO: 386 - protein, 15 aa, Homo sapiens
CASRTGTFFNQPQHF

SEQ ID NO: 387 - protein, 12 aa, Homo sapiens
CASSTGNQPQHF

SEQ ID NO: 388 - protein, 17 aa, Homo sapiens
CASSAPEGGGFTDTQYF

SEQ ID NO: 389 - protein, 17 aa, Homo sapiens
CASSTRQGTTNTGELFF

SEQ ID NO: 390 - protein, 15 aa, Homo sapiens
CASSATTGFSEKLFF

SEQ ID NO: 391 - protein, 13 aa, Homo sapiens
CASSTTGYGELFF

SEQ ID NO: 392 - protein, 13 aa, Homo sapiens
CASSFGTAGTQYF

SEQ ID NO: 393 - protein, 13 aa, Homo sapiens
CASSWGTDYEQYF

SEQ ID NO: 394 - protein, 12 aa, Homo sapiens
CASSGGNQPQHF

SEQ ID NO: 395 - protein, 15 aa, Homo sapiens
CASSYGTLDPYGYTF

SEQ ID NO: 396 - protein, 14 aa, Homo sapiens
CASSGQATSYEQYF

SEQ ID NO: 397 - protein, 14 aa, Homo sapiens
CASSYSANNYGYTF

SEQ ID NO: 398 - protein, 16 aa, Homo sapiens
CASSHSRDRVGEKLFF

SEQ ID NO: 399 - protein, 12 aa, Homo sapiens
CASSYTGLEQYF

SEQ ID NO: 400 - protein, 16 aa, Homo sapiens
CASSIAEGTIYNEQFF

SEQ ID NO: 401 - protein, 13 aa, Homo sapiens
CASSYVGGAEAFF

SEQ ID NO: 402 - protein, 14 aa, Homo sapiens
CASSIPGRRETQYF

SEQ ID NO: 403 - protein, 14 aa, Homo sapiens
CASSYVREDYGYTF

SEQ ID NO: 404 - protein, 12 aa, Homo sapiens
CASSISSDGYTF

SEQ ID NO: 405 - protein, 13 aa, Homo sapiens
CASTGGYNSPLHF

SEQ ID NO: 406 - protein, 15 aa, Homo sapiens
CASSLDEGYTGELFF

SEQ ID NO: 407 - protein, 11 aa, Homo sapiens
CASTLGGEQYF

SEQ ID NO: 408 - protein, 14 aa, Homo sapiens
CASSLDSSNQPQHF

SEQ ID NO: 409 - protein, 12 aa, Homo sapiens
CASVQGNTEAFF

SEQ ID NO: 410 - protein, 14 aa, Homo sapiens
CASSLGAGANVLTF

SEQ ID NO: 411 - protein, 15 aa, Homo sapiens
CATSTYEGADQPQHF

SEQ ID NO: 412 - protein, 14 aa, Homo sapiens
CASSLGGLAGEQYF

SEQ ID NO: 413 - protein, 13 aa, Homo sapiens
CAWSVGGIQPQHF

SEQ ID NO: 414 - protein, 11 aa, Homo sapiens
CASSLGGQLFF

SEQ ID NO: 415 - protein, 13 aa, Homo sapiens
CSAPGHLNYGYTF

SEQ ID NO: 416 - protein, 13 aa, Homo sapiens
CASSLGLAGEQYF

SEQ ID NO: 417 - protein, 15 aa, Homo sapiens
CSARDGGGDWEKLFF

SEQ ID NO: 418 - protein, 13 aa, Homo sapiens
CASSLGSKSTQYF

SEQ ID NO: 419 - protein, 15 aa, Homo sapiens
CSAREGQFSGNTIYF

SEQ ID NO: 420 - protein, 14 aa, Homo sapiens
CASSNPGLQETQYF

SEQ ID NO: 421 - protein, 15 aa, Homo sapiens
CASSYFGGPGNTIYF

SEQ ID NO: 422 - protein, 16 aa, Homo sapiens
CASSNDRARAKNIQYF

SEQ ID NO: 423 - protein, 17 aa, Homo sapiens
CASSQDRRSSYNSPLHF

SEQ ID NO: 424 - protein, 15 aa, Homo sapiens
CASSLVQDVGDEAFF

SEQ ID NO: 425 - protein, 16 aa, Homo sapiens
CASSQDLQTTFYEQYF

SEQ ID NO: 426 - protein, 15 aa, Homo sapiens
CASSLTSGPLYEQFF

SEQ ID NO: 427 - protein, 14 aa, Homo sapiens
CASSAVGGAYEQYF

SEQ ID NO: 428 - protein, 14 aa, Homo sapiens
CASSLRGGGETQYF

SEQ ID NO: 429 - protein, 12 aa, Homo sapiens
CSVTTGGQEAFF

SEQ ID NO: 430 - protein, 14 aa, Homo sapiens
CASSLNLGNTEAFF

SEQ ID NO: 431 - protein, 16 aa, Homo sapiens
CSVALGAVRSTDTQYF

SEQ ID NO: 432 - protein, 11 aa, Homo sapiens
CASSLGETQYF

SEQ ID NO: 433 - protein, 16 aa, Homo sapiens
CSARDIVNANTGELFF

SEQ ID NO: 434 - protein, 13 aa, Homo sapiens
CASSLFMGWEQYF

SEQ ID NO: 435 - protein, 13 aa, Homo sapiens
CSAPIQAGTEAFF

SEQ ID NO: 436 - protein, 16 aa, Homo sapiens
CASSLEAGTLDYGYTF

SEQ ID NO: 437 - protein, 15 aa, Homo sapiens
CATSRDRSWDSPLHF

SEQ ID NO: 438 - protein, 15 aa, Homo sapiens
CASSIGTGGPYEQYF

SEQ ID NO: 439 - protein, 14 aa, Homo sapiens
CASTNGGMNTEAFF

SEQ ID NO: 440 - protein, 15 aa, Homo sapiens
CASSHPSSPHEKLFF

SEQ ID NO: 441 - protein, 15 aa, Homo sapiens
CASSYSVGNTGELFF

SEQ ID NO: 442 - protein, 14 aa, Homo sapiens
CASSFSGGAGEQFF

SEQ ID NO: 443 - protein, 13 aa, Homo sapiens
CASSVGVVYEQYF

SEQ ID NO: 444 - protein, 17 aa, Homo sapiens
CASSESGTGIGSQPQHF

SEQ ID NO: 445 - protein, 15 aa, Homo sapiens
CASSSSTVSGNTIYF

SEQ ID NO: 446 - protein, 15 aa, Homo sapiens
CASSELTRGTDTQYF

SEQ ID NO: 447 - protein, 18 aa, Homo sapiens
CASSQQAPTSSYNSPLHF

SEQ ID NO: 448 - protein, 17 aa, Homo sapiens
CASSDGLAGFSTDTQYF

SEQ ID NO: 449 - protein, 15 aa, Homo sapiens
CASSQDLSWESPLHF

SEQ ID NO: 450 - protein, 11 aa, Homo sapiens
CASRLTDTQYF

SEQ ID NO: 451 - protein, 17 aa, Homo sapiens
CASSPVLGAFFGYGYTF

SEQ ID NO: 452 - protein, 14 aa, Homo sapiens
CASQQTGGFNEQYF

SEQ ID NO: 453 - protein, 15 aa, Homo sapiens
CASSPTWTGGNEQFF

SEQ ID NO: 454 - protein, 14 aa, Homo sapiens
CASKTGVSYNEQFF

SEQ ID NO: 455 - protein, 16 aa, Homo sapiens
CASSPDRPPIYNEQFF

SEQ ID NO: 456 - protein, 14 aa, Homo sapiens
CASGGGMGGQPQHF

SEQ ID NO: 457 - protein, 16 aa, Homo sapiens
CASSPAVAGGRDTQYF

SEQ ID NO: 458 - protein, 11 aa, Homo sapiens
CAGTTGYEQYF

SEQ ID NO: 459 - protein, 19 aa, Homo sapiens
CALGGLPTLGDTPTDKLIF

SEQ ID NO: 460 - protein, 17 aa, Homo sapiens
CALCLLADWGYTDKLIF

SEQ ID NO: 461 - protein, 19 aa, Homo sapiens
CALAPLPTLGDTGPDKLIF

SEQ ID NO: 462 - protein, 28 aa, Homo sapiens
CALGDGGGFYTSRVLGGYAFVTTDKLIF

SEQ ID NO: 463 - protein, 19 aa, Homo sapiens
CALGELPYWALRGADKLIF

SEQ ID NO: 464 - protein, 12 aa, Homo sapiens
CALGGYADKLIF

SEQ ID NO: 465 - protein, 17 aa, Homo sapiens
CASSPSYGGYAVDKLIF

SEQ ID NO: 466 - protein, 18 aa, Homo sapiens
CACDTLLGDTLLTAQLFF

SEQ ID NO: 467 - protein, 14 aa, Homo sapiens
CACDTLLGENKLIF

SEQ ID NO: 468 - protein, 15 aa, Homo sapiens
CAFNRGLLYTDKLIF

SEQ ID NO: 469 - protein, 21 aa, Homo sapiens
CALGAFLPRYWGPRHTDKLIF

SEQ ID NO: 470 - protein, 23 aa, Homo sapiens
CALGDPSLPLNWGIRGHGIQLIF

SEQ ID NO: 471 - protein, 21 aa, Homo sapiens
CALGEARPSYWGIRTTDKLIF

SEQ ID NO: 472 - protein, 21 aa, Homo sapiens
CALGEFFPRYWGTTYTDKLIF

SEQ ID NO: 473 - protein, 25 aa, Homo sapiens
CALGEKPPFLSKVLGDTHYTDKLIF

SEQ ID NO: 474 - protein, 19 aa, Homo sapiens
CALGELIGWGPKDADKLIF

SEQ ID NO: 475 - protein, 22 aa, Homo sapiens
CALGELQPRYWGRRFDKTKLFF

SEQ ID NO: 476 - protein, 23 aa, Homo sapiens
CALGELRSLLHLHWGIRTDKLIF

SEQ ID NO: 477 - protein, 21 aa, Homo sapiens
CALGERFRGYWGIQYTDKLIF

SEQ ID NO: 478 - protein, 22 aa, Homo sapiens
CALGERHPSYWGNKGHTDKLIF

SEQ ID NO: 479 - protein, 19 aa, Homo sapiens
CALGFPPVLGDPYTDKLIF

SEQ ID NO: 480 - protein, 19 aa, Homo sapiens
CALGGSGISYVGILGKLIF

SEQ ID NO: 481 - protein, 21 aa, Homo sapiens
CALGGTSYVPWGIVRRDKLIF

SEQ ID NO: 482 - protein, 20 aa, Homo sapiens
CALGKGGNGVYWGSTRPLIF

SEQ ID NO: 483 - protein, 20 aa, Homo sapiens
CALGNEAFRLVLGETDKLIF

SEQ ID NO: 484 - protein, 22 aa, Homo sapiens
CALGPLSTPPYWGILGTDKLIF

SEQ ID NO: 485 - protein, 22 aa, Homo sapiens
CALGPRFLRGVVGIRYTDKLIF

SEQ ID NO: 486 - protein, 20 aa, Homo sapiens
CALGVLPTLGDQGPTDKLIF

SEQ ID NO: 487 - protein, 24 aa, Homo sapiens
CALRGGRSPVLGDTLKRRTDKLIF

SEQ ID NO: 488 - protein, 21 aa, Homo sapiens
CALGERSPSYWGPHFTDKLIF

SEQ ID NO: 489 - protein, 16 aa, Homo sapiens
CAASAGGPQTTDKLIF

SEQ ID NO: 490 - protein, 20 aa, Homo sapiens
CALGELVRSYFGIRGGKLIF

SEQ ID NO: 491 - protein, 21 aa, Homo sapiens
CALGERLPNYWGTLYTDKLIF

SEQ ID NO: 492 - protein, 20 aa, Homo sapiens
CALGERRPSYWGIRRGPLIF

SEQ ID NO: 493 - protein, 15 aa, Homo sapiens
CAASPLVGNTDKLIF

SEQ ID NO: 494 - protein, 15 aa, Homo sapiens
CACDTVGIQSDKLIF

SEQ ID NO: 495 - protein, 18 aa, Homo sapiens
CALGDPPSRGARPDKLIF

SEQ ID NO: 496 - protein, 24 aa, Homo sapiens
CALGDPTGPYWGKYYLSYTDKLIF

SEQ ID NO: 497 - protein, 22 aa, Homo sapiens
CALGECHPSYWGRPINTDKLIF

SEQ ID NO: 498 - protein, 24 aa, Homo sapiens
CALGECPTRHPTGGYIPYTDKLIF

SEQ ID NO: 499 - protein, 21 aa, Homo sapiens
CALGELRPSYVFGGYAYKLIF

SEQ ID NO: 500 - protein, 21 aa, Homo sapiens
CALGELSPRYWGIGYTDKLIF

SEQ ID NO: 501 - protein, 23 aa, Homo sapiens
CALGELSRPADWGILIYTDKLIF

SEQ ID NO: 502 - protein, 22 aa, Homo sapiens
CALGEPTGAFLLTGGFTDKLIF

SEQ ID NO: 503 - protein, 21 aa, Homo sapiens
CALGEQNPRYWGASYTDKLIF

SEQ ID NO: 504 - protein, 21 aa, Homo sapiens
CALGERLPSYWGISYTDKLIF

SEQ ID NO: 505 - protein, 22 aa, Homo sapiens
CALGESYVSYWGGYLYTDKLIF

SEQ ID NO: 506 - protein, 21 aa, Homo sapiens
CALGEYLPRYWGIHGTDKLIF

SEQ ID NO: 507 - protein, 22 aa, Homo sapiens
CALGELPLLGDTLRSYTDKLIF

SEQ ID NO: 508 - protein, 21 aa, Homo sapiens
CALGERGPRYWGIAYTDKLIF

SEQ ID NO: 509 - protein, 27 aa, Homo sapiens
CALGVPLLQVKLGDTKGLLGDTDKLIF

SEQ ID NO: 510 - protein, 17 aa, Homo sapiens
CALGERFPWPHTDKLIF

SEQ ID NO: 511 - protein, 15 aa, Homo sapiens
CALGNIWGVTDKLIF

SEQ ID NO: 512 - protein, 23 aa, Homo sapiens
CALGEPSDSAYWGIRGNTDKLIF

SEQ ID NO: 513 - protein, 20 aa, Homo sapiens
CALGELVLRYWGGRMDKLIF

SEQ ID NO: 514 - protein, 20 aa, Homo sapiens
CALGEPRAVLGDTLGDKLIF

SEQ ID NO: 515 - protein, 17 aa, Homo sapiens
CALGEGAGILTGDKLIF

SEQ ID NO: 516 - protein, 21 aa, Homo sapiens
CALGEPLPSYWGPRGSDKLIF

SEQ ID NO: 517 - protein, 13 aa, Homo sapiens
CAFLRIRPDKLIF

SEQ ID NO: 518 - protein, 24 aa, Homo sapiens
CALGEPDSTFVRGGYAGNTDKLIF

SEQ ID NO: 519 - protein, 22 aa, Homo sapiens
CALVSNPPPRYPGVRDTDKLIF

SEQ ID NO: 520 - protein, 19 aa, Homo sapiens
CALGEPDLPTTWYTDKLIF

SEQ ID NO: 521 - protein, 17 aa, Homo sapiens
CALVADYWGIGTDKLIF

SEQ ID NO: 522 - protein, 17 aa, Homo sapiens
CALGEPALQLGVNKLIF

SEQ ID NO: 523 - protein, 19 aa, Homo sapiens
CALGPRFLRRGIRADKLIF

SEQ ID NO: 524 - protein, 22 aa, Homo sapiens
CALGENPPPYLGGYPYTDKLIF

SEQ ID NO: 525 - protein, 21 aa, Homo sapiens
CALGPRAHQRTGDRVTAQLFF

SEQ ID NO: 526 - protein, 22 aa, Homo sapiens
CALGENFPSSWGIHRYTDKLIF

SEQ ID NO: 527 - protein, 25 aa, Homo sapiens
CALGNPKGTSYGLRGIPPYTDKLIF

SEQ ID NO: 528 - protein, 22 aa, Homo sapiens
CALGELVYPGGYYGRETAQLFF

SEQ ID NO: 529 - protein, 25 aa, Homo sapiens
CALGKGGSYVVHFYWGIESTDKLIF

SEQ ID NO: 530 - protein, 16 aa, Homo sapiens
CALGELVFLLRAGLIF

SEQ ID NO: 531 - protein, 20 aa, Homo sapiens
CALGISLLRLGDMISDKLIF

SEQ ID NO: 532 - protein, 23 aa, Homo sapiens
CALGELSDLQCVLGDRPTRPLIF

SEQ ID NO: 533 - protein, 20 aa, Homo sapiens
CALGEPPFLRRYRYTDKLIF

SEQ ID NO: 534 - protein, 20 aa, Homo sapiens
CALGELRRIYWGIRIDKLIF

SEQ ID NO: 535 - protein, 19 aa, Homo sapiens
CALGELPTWTYWGIDKLIF

SEQ ID NO: 536 - protein, 21 aa, Homo sapiens
CALGELLPRYWGIGGTDKLIF

SEQ ID NO: 537 - protein, 21 aa, Homo sapiens
CALGELIRGYWGIRYTDKLIF

SEQ ID NO: 538 - protein, 30 aa, Homo sapiens
CALGELLASETYTGGSVVLYRARKTDKLIF

SEQ ID NO: 539 - protein, 20 aa, Homo sapiens
CALGDPPPHRSLLYRYKLIF

SEQ ID NO: 540 - protein, 20 aa, Homo sapiens
CALGEKGNMPLGDIIDKLIF

SEQ ID NO: 541 - protein, 19 aa, Homo sapiens
CALGALPTLGDRGVDKLIF

SEQ ID NO: 542 - protein, 21 aa, Homo sapiens
CALGEHEVHPGGYWYTDKLIF

SEQ ID NO: 543 - protein, 22 aa, Homo sapiens
CALDTPKHSSGGYFKRTDKLIF

SEQ ID NO: 544 - protein, 24 aa, Homo sapiens
CALGEGTGDFGRWGILVYTDKLIF

SEQ ID NO: 545 - protein, 15 aa, Homo sapiens
CASHFLRAGYAKLIF

SEQ ID NO: 546 - protein, 21 aa, Homo sapiens
CALGEGPFLRTGGLYTDKLIF

SEQ ID NO: 547 - protein, 15 aa, Homo sapiens
CASCPGVGDNDKLIF

SEQ ID NO: 548 - protein, 24 aa, Homo sapiens
CALGEGPAPIWGIRRRSYTDKLIF

SEQ ID NO: 549 - protein, 19 aa, Homo sapiens
CAQTTYWGMGGQYTDKLIF

SEQ ID NO: 550 - protein, 17 aa, Homo sapiens
CALGEFYWGILSDKLIF

SEQ ID NO: 551 - protein, 12 aa, Homo sapiens
CAPFSWPDKLIF

SEQ ID NO: 552 - protein, 18 aa, Homo sapiens
CALGEEIPTGGYPDKLIF

SEQ ID NO: 553 - protein, 17 aa, Homo sapiens
CALVSGGFPSYADKLIF

SEQ ID NO: 554 - protein, 22 aa, Homo sapiens
CALGEDPSFLRLGIRYTDKLIF

SEQ ID NO: 555 - protein, 15 aa, Homo sapiens
CALRWGIRGADKLIF

SEQ ID NO: 556 - protein, 23 aa, Homo sapiens
CALGDWRSSYFNWGISSPDKLIF

SEQ ID NO: 557 - protein, 17 aa, Homo sapiens
CALRVFTYWGDTDKLIF

SEQ ID NO: 558 - protein, 22 aa, Homo sapiens
CALGDPSEEAHTGGYNTDKLIF

SEQ ID NO: 559 - protein, 24 aa, Homo sapiens
CALGVRIFPPSLLGDTGYGGVLIF

SEQ ID NO: 560 - protein, 19 aa, Homo sapiens
CALGDLLGLPRGPTDKLIF

SEQ ID NO: 561 - protein, 19 aa, Homo sapiens
CALGSLLINWGIVTDKLIF

SEQ ID NO: 562 - protein, 18 aa, Homo sapiens
CALGDFPTWGGVPDKLIF

SEQ ID NO: 563 - protein, 19 aa, Homo sapiens
CALGSGAYPYRTGGRELIF

SEQ ID NO: 564 - protein, 17 aa, Homo sapiens
CALGASLGDNSPDKLIF

SEQ ID NO: 565 - protein, 20 aa, Homo sapiens
CALGPPPFLIGSWDTRQMFF

SEQ ID NO: 566 - protein, 23 aa, Homo sapiens
CALGALGSLPTHWGIRATDKLIF

SEQ ID NO: 567 - protein, 18 aa, Homo sapiens
CALGPGAFLRSWGQKLIF

SEQ ID NO: 568 - protein, 20 aa, Homo sapiens
CALGALGLRGSLGVYRKLIF

SEQ ID NO: 569 - protein, 18 aa, Homo sapiens
CALGNSYWGIPYTDKLIF

SEQ ID NO: 570 - protein, 13 aa, Homo sapiens
CALEAPYTDKLIF

SEQ ID NO: 571 - protein, 13 aa, Homo sapiens
CALGNHWADKLIF

SEQ ID NO: 572 - protein, 22 aa, Homo sapiens
CALAQPSSNLLIHWGILDKLIF

SEQ ID NO: 573 - protein, 19 aa, Homo sapiens
CALGLPIGLGDSYLYKLIF

SEQ ID NO: 574 - protein, 14 aa, Homo sapiens
CAITGSKGTDKLIF

SEQ ID NO: 575 - protein, 23 aa, Homo sapiens
CALGKRPYPLYWGIRGYTDKLIF

SEQ ID NO: 576 - protein, 21 aa, Homo sapiens
CAFRHGPNYPLIYWGISKLIF

SEQ ID NO: 577 - protein, 16 aa, Homo sapiens
CALGFYWGEYTDKLIF

SEQ ID NO: 578 - protein, 15 aa, Homo sapiens
CAFRGLWGYTDKLIF

SEQ ID NO: 579 - protein, 19 aa, Homo sapiens
CALGEYSRLTGVYTDKLIF

SEQ ID NO: 580 - protein, 17 aa, Homo sapiens
  misc_feature (5)..(5): Xaa can be any naturally occurring amino acid
CAFPXWGHSLYTDKLIF

SEQ ID NO: 581 - protein, 25 aa, Homo sapiens
  misc_feature (15)..(15): Xaa can be any naturally occurring amino acid
CALGEYPPLGDTFVXTTXDTRQMFF

SEQ ID NO: 582 - protein, 15 aa, Homo sapiens
CAFLALPMYTDKLIF

SEQ ID NO: 583 - protein, 23 aa, Homo sapiens
CALGEWFPGYFLTKFRNTDKLIF

SEQ ID NO: 584 - protein, 21 aa, Homo sapiens
CACVKAFLKRGDTPYTDKLIF

SEQ ID NO: 585 - protein, 20 aa, Homo sapiens
CALGESVRWVFGEYTDKLIF

SEQ ID NO: 586 - protein, 18 aa, Homo sapiens
CACTFLGLGGSNTDKLIF

SEQ ID NO: 587 - protein, 21 aa, Homo sapiens
CALGERYPKYWGAPGTDKLIF

SEQ ID NO: 588 - protein, 16 aa, Homo sapiens
CACETWGIKGTDKLIF

SEQ ID NO: 589 - protein, 23 aa, Homo sapiens
CALGERSYVPYWGTGRGTDKLIF

SEQ ID NO: 590 - protein, 16 aa, Homo sapiens
CACERGGYAFTDKLIF

SEQ ID NO: 591 - protein, 21 aa, Homo sapiens
  misc_feature (14)..(14): Xaa can be any naturally occurring amino acid
CALGERIPTSWGIXYTDKLIF

SEQ ID NO: 592 - protein, 20 aa, Homo sapiens
CACDSRTSTWGIRMADKLIF

SEQ ID NO: 593 - protein, 18 aa, Homo sapiens
CALGELPPPGGYFDKLIF

SEQ ID NO: 594 - protein, 15 aa, Homo sapiens
CAFKGLLGGSVGLIF

SEQ ID NO: 595 - protein, 22 aa, Homo sapiens
CALGDSSLGGWGILSSTDKLIF

SEQ ID NO: 596 - protein, 18 aa, Homo sapiens
CALGVLHWGNSLTAQLFF

SEQ ID NO: 597 - protein, 20 aa, Homo sapiens
CALPFSYWGIRLVGTDKLIF

SEQ ID NO: 598 - protein, 15 aa, Homo sapiens
CASTGAVGKSPKLIF

SEQ ID NO: 599 - protein, 19 aa, Homo sapiens
CAASAGLPGGLGYTDKLIF

SEQ ID NO: 600 - protein, 15 aa, Homo sapiens
CAASALRGSFDKLIF

SEQ ID NO: 601 - protein, 17 aa, Homo sapiens
CACDHDYGTGGVRKLIF

SEQ ID NO: 602 - protein, 19 aa, Homo sapiens
CACRLPTRWGIGYTDKLIF

SEQ ID NO: 603 - protein, 15 aa, Homo sapiens
CACRPSYGGIVKLIF

SEQ ID NO: 604 - protein, 19 aa, Homo sapiens
CAFILTIYGPGGITDKLIF

SEQ ID NO: 605 - protein, 17 aa, Homo sapiens
CAFPTGGLLGDTDKLIF

SEQ ID NO: 606 - protein, 14 aa, Homo sapiens
CAFVGGPYTDKLIF

SEQ ID NO: 607 - protein, 17 aa, Homo sapiens
CALADLRPGGYSAQLFF

SEQ ID NO: 608 - protein, 18 aa, Homo sapiens
CALEVVHHPIRYTDKLIF

SEQ ID NO: 609 - protein, 21 aa, Homo sapiens
CALGAHLRNYWGPLYTDKLIF

SEQ ID NO: 610 - protein, 20 aa, Homo sapiens
CALGAYPPGGTGRYTDKLIF

SEQ ID NO: 611 - protein, 21 aa, Homo sapiens
CALGDFLPSYWGIRGTDKLIF

SEQ ID NO: 612 - protein, 20 aa, Homo sapiens
CALGDPFQNYQGPYTDKLIF

SEQ ID NO: 613 - protein, 20 aa, Homo sapiens
CALGEAFLSYWGTNHDKLIF

SEQ ID NO: 614 - protein, 20 aa, Homo sapiens
CALGEGGGVLRNPYTDKLIF

SEQ ID NO: 615 - protein, 23 aa, Homo sapiens
CALGEHGAAFLPYWGIRRGKLIF

SEQ ID NO: 616 - protein, 21 aa, Homo sapiens
CALGEIYRGYWGIRAGDKLIF

SEQ ID NO: 617 - protein, 18 aa, Homo sapiens
CALGELHWGTRYTDKLIF

SEQ ID NO: 618 - protein, 17 aa, Homo sapiens
CALGELLRTGGLAQLFF

SEQ ID NO: 619 - protein, 20 aa, Homo sapiens
CALGELMLGRWGEYTDKLIF

SEQ ID NO: 620 - protein, 23 aa, Homo sapiens
CALGELNLPQYWGPLVGTDKLIF

SEQ ID NO: 621 - protein, 19 aa, Homo sapiens
CALGELPPWGIPYTDKLIF

SEQ ID NO: 622 - protein, 25 aa, Homo sapiens
CALGELRLRWMGDTLFLQLTDKLIF

SEQ ID NO: 623 - protein, 22 aa, Homo sapiens
CALGELRRGIRGQRIGTDKLIF

SEQ ID NO: 624 - protein, 23 aa, Homo sapiens
CALGELSRPSYYYDPSYTDKLIF

SEQ ID NO: 625 - protein, 21 aa, Homo sapiens
CALGELSSPHTGGYYTDKLIF

SEQ ID NO: 626 - protein, 21 aa, Homo sapiens
CALGELSYRGGWGIRADKLIF

SEQ ID NO: 627 - protein, 22 aa, Homo sapiens
CALGENKFVFGGLIVLTAQLFF

SEQ ID NO: 628 - protein, 21 aa, Homo sapiens
CALGEPIGPPLLGVYTDKLIF

SEQ ID NO: 629 - protein, 24 aa, Homo sapiens
CALGEPQTFLPRYWGGTYTDKLIF

SEQ ID NO: 630 - protein, 16 aa, Homo sapiens
CALGEPSTGGSDKLIF

SEQ ID NO: 631 - protein, 18 aa, Homo sapiens
CALGEQWILRGDTDKLIF

SEQ ID NO: 632 - protein, 19 aa, Homo sapiens
CALGERLRGYALKTDKLIF

SEQ ID NO: 633 - protein, 17 aa, Homo sapiens
CALGERLSPYYTDKLIF

SEQ ID NO: 634 - protein, 21 aa, Homo sapiens
CALGERPSYGWGFGWTDKLIF

SEQ ID NO: 635 - protein, 21 aa, Homo sapiens
CALGETTLSYWGIRYTDKLIF

SEQ ID NO: 636 - protein, 22 aa, Homo sapiens
CALGGGLPTSGGYRSYTDKLIF

SEQ ID NO: 637 - protein, 26 aa, Homo sapiens
CALGHRAPSRAQPYWGILAYTDKLIF

SEQ ID NO: 638 - protein, 21 aa, Homo sapiens
CALGKPAKSYWGMRYTDKLIF

SEQ ID NO: 639 - protein, 20 aa, Homo sapiens
CALGPLPGGYSSWDTRQMFF

SEQ ID NO: 640 - protein, 22 aa, Homo sapiens
CALGQRIPSYWGIAGSTDKLIF

SEQ ID NO: 641 - protein, 21 aa, Homo sapiens
CALGVISPSYWGPQYTDKLIF

SEQ ID NO: 642 - protein, 19 aa, Homo sapiens
CALGVSSSAGDLLTDKLIF

SEQ ID NO: 643 - protein, 16 aa, Homo sapiens
CALKPGGYSLTDKLIF

SEQ ID NO: 644 - protein, 14 aa, Homo sapiens
CALMAGPYTDKLIF

SEQ ID NO: 645 - protein, 19 aa, Homo sapiens
CASVCYGNGHISRLDKLIF

SEQ ID NO: 646 - DNA, 24 bp, Artificial Sequence
  TRDV1 PCR primer, reaction 1
ccagggttctgatgaacagaatgc

SEQ ID NO: 647 - DNA, 25 bp, Artificial Sequence
  TRDV2 PCR primer, reaction 1
cctggtttcaaagacaatttccaag

SEQ ID NO: 648 - DNA, 24 bp, Artificial Sequence
  TRDV3 PCR primer, reaction 1
ggataacagcagatcagaaggtgc

SEQ ID NO: 649 - DNA, 24 bp, Artificial Sequence
  TRDV4 PCR primer, reaction 1
gcaaaatgcaacagaaggtcgcta

SEQ ID NO: 650 - DNA, 27 bp, Artificial Sequence
  TRDV5 PCR primer, reaction 1
ggataaaaatgaagatggaagattcac

SEQ ID NO: 651 - DNA, 25 bp, Artificial Sequence
  TRDV6 PCR primer, reaction 1
ccagatgtgagtgaaaagaaagaag

SEQ ID NO: 652 - DNA, 26 bp, Artificial Sequence
  TRDV7 PCR primer, reaction 1
gctaacttcaagtggaattgaaaaga

SEQ ID NO: 653 - DNA, 26 bp, Artificial Sequence
  TRDV8 PCR primer, reaction 1
gaagcttataagcaacagaatgcaac

SEQ ID NO: 654 - DNA, 48 bp, Artificial Sequence
  TRDV1 PCR primer, reaction 2
gcatacgagctcttccgatctgagtggtcgctattctgtcaacttcaa

SEQ ID NO: 655 - DNA, 49 bp, Artificial Sequence
  TRDV2 PCR primer, reaction 2
gcatacgagctcttccgatctgagtgacattgatattgcaaagaacctg

SEQ ID NO: 656 - DNA, 48 bp, Artificial Sequence
  TRDV3 PCR primer, reaction 2
gcatacgagctcttccgatctgaggacggttttctgtgaaacacattc

SEQ ID NO: 657 - DNA, 47 bp, Artificial Sequence
  TRDV4 PCR primer, reaction 2
gcatacgagctcttccgatctgatccagaaggcaagaaaatccgcca

SEQ ID NO: 658 - DNA, 48 bp, Artificial Sequence
  TRDV5 PCR primer, reaction 2
gcatacgagctcttccgatctgacttaaacaaaagtgccaagcacctc

SEQ ID NO: 659 - DNA, 49 bp, Artificial Sequence
  TRDV6 PCR primer, reaction 2
gcatacgagctcttccgatctgacacaatctccttcaataaaagtgcca

SEQ ID NO: 660 - DNA, 50 bp, Artificial Sequence
  TRDV7 PCR primer, reaction 2
gcatacgagctcttccgatctgaggaagactaagtagcatattagataag

SEQ ID NO: 661 - DNA, 47 bp, Artificial Sequence
  TRDV8 PCR primer, reaction 2
gcatacgagctcttccgatctgactgtgaacttccagaaagcagcca

SEQ ID NO: 662 - DNA, 23 bp, Artificial Sequence
  5′ primer, reaction 3
gcatacgagctcttccgatctga

SEQ ID NO: 663 - DNA, 26 bp, Artificial Sequence
  TRDC primer, reaction 1
cgagatttattcttatatccttgggg

SEQ ID NO: 664 - DNA, 24 bp, Artificial Sequence
  TRDC primer, reaction 2
ccttcaccagacaagcgacatttg

SEQ ID NO: 665 - DNA, 26 bp, Artificial Sequence
  TRDC primer, reaction 3
catttttcatgacaaaaacggatggt

SEQ ID NO: 666 - protein, 7 aa, Homo sapiens
  misc_feature (2)..(5): Xaa can be any naturally occurring amino acid
CXXXXGN

SEQ ID NO: 667 - protein, 7 aa, Homo sapiens
  misc_feature (2)..(5): Xaa can be any naturally occurring amino acid
CXXXXGT

SEQ ID NO: 668 - protein, 5 aa, Homo sapiens
  misc_feature (2)..(4): Xaa can be any naturally occurring amino acid
CXXXF

(19) United States
(12) Patent Application Publication
(10) Pub. No.: US 2016/0091491 A1
(43) Pub. Date: Mar. 31, 2016

(54) METHODS FOR DIAGNOSIS OF CELIAC DISEASE

(71) Applicant: THE BOARD OF TRUSTEES OF THE LELAND STANFORD JUNIOR UNIVERSITY, Palo Alto, CA (US)

(72) Inventors: Mark M. Davis, Atherton, CA (US); Arnold Han, Los Altos Hills, CA (US); Evan W. Newell, Singapore 276750 (SG)

(21) Appl. No.: 14/785,335

(22) PCT Filed: Apr. 28, 2014
(86) PCT No.: PCT/US14/35643

§ 371 Date: Oct. 17, 2015

Related U.S. Application Data
(60) Provisional application No. 61/818,538, filed on May 2, 2013.

(51) Int. Cl. G01N 33/569

(52) U.S. Cl. CPC G01N 33/56972; G01N 2469/10; G01N 2333/70517; G01N 2333/7051; G01N 2333/70514; G01N 2333/70596; G01N 2333/70546; G01N 2800/065

(57) ABSTRACT

A diagnostic test for celiac disease is disclosed. In particular, the invention relates to a method of diagnosing celiac disease by detecting activated, gut-bound CD8+ alpha-beta T lymphocytes and gamma-delta T lymphocytes in the peripheral blood of a subject who has consumed gluten for one to three days. This diagnostic test has a number of advantages over current tests for celiac test, including that the test is noninvasive, relatively inexpensive, and requires voluntary gluten ingestion over a short period of time.

### STATEMENT REGARDING FEDERALLY SPONSORED RESEARCH OR DEVELOPMENT

This invention was made with Government support under contracts AI057229 and AI090019 awarded by the National Institutes of Health. The Government has certain rights in this invention.

### TECHNICAL FIELD

The present invention pertains generally to methods for diagnosis of celiac disease. In particular, the invention relates to methods of diagnosing celiac disease by detecting activated CD8+ alpha-beta T lymphocytes and gamma-delta T lymphocytes in the peripheral blood of a subject who has consumed gluten for 1 to 3 days.

### BACKGROUND

Celiac disease (CD) is a common incurable autoimmune disease with an estimated prevalence of 1% in the Western world. It is characterized by small intestinal mucosal injury and nutrient malabsorption in genetically susceptible individuals due to dietary gluten ingestion. There is a strong association with human leukocyte antigen (HLA)-class II molecules DQ2 and DQ8 and the CD4⁺ T cell response is known to be essential in CD. CD-associated gluten peptide CD4⁺ T cell epitopes have been discovered, and gluten-reactive CD4⁺ T cells have been identified in the tissue and blood of individuals with CD. While CD4⁺ T cells are indispensable to CD, mouse studies have shown that a gluten-specific CD4+ T cell response is not sufficient to induce intestinal tissue damage (De Kauwe et al. (2009) J. Immunol. 182:7440-7450). Mucosal damage is primarily driven by intestinal intraepithelial lymphocytes (IELs), including CD8⁺TCR αβ⁺ IELs (CD8 T-IEL), which extensively infiltrate celiac intestinal lesions (Jabri & Sollid (2009) Nat. Rev. Immunol. 9:858-870). The function of TCR γδ⁺ IEL (GD-IEL) in health or in CD is unclear, although an increase in GD-IEL is a hallmark of all stages of CD and persists even in the presence of a gluten-free diet (GFD) (Meresse & Cerf-Bensussan (2009) Semin. Immunol. 21:121-129). The means through which dietary gluten enables recruitment and activation of IELs, in a manner presumably dependent upon gluten-specific CD4⁺ cells, has long remained elusive.

Celiac disease is currently diagnosed with serological blood tests for anti-endomysial, anti-transglutaminase-2 (TG2), or anti-gliadin antibodies and endoscopy with biopsy of the duodenum or jejunum (Lindfors et al. (2011) Int. Rev. Immunol. 30(4):185-196; Walker et al. (2011) Histopathology 59(2):166-179). These methods of testing for celiac disease are most accurate when the disease is severe, but become unreliable when the disease is inactive. For that matter, these tests often fail to detect celiac disease if a patient is already on a gluten-free diet because autoimmune antibody levels decline and intestinal damage heals after removal of gluten from the diet. Currently an estimated 1.6 million Americans follow a gluten-free diet without an established diagnosis of celiac disease (Rubio-Tapia et al. (2012) Am. J. Gastroenterol. 107:1538-1544). Thus, in order to confirm a diagnosis of celiac disease by these methods, the patient needs to voluntarily consume gluten-containing food for a month or longer before performing the diagnostic tests, which many patients tolerate poorly and are unwilling to do.

Therefore, there remains a need for more convenient, sensitive diagnostic tests for celiac disease that do not require an extended period of gluten consumption.

### SUMMARY

The invention relates to a method of diagnosing celiac disease by detecting activated, gut-bound CD8+ αβ T lymphocytes and γδ T lymphocytes in the peripheral blood of a subject who has consumed gluten for one to three days. This diagnostic method has a number of advantages over current tests for celiac test, including that the method is noninvasive, relatively inexpensive, and requires voluntary gluten ingestion over a short period of time.

In one aspect, the invention includes a method for diagnosing celiac disease in a subject, the method comprising: a) obtaining a blood sample comprising peripheral blood lymphocytes from the subject after the subject has consumed gluten for 1 to 3 days; and b) measuring the levels of activated, gut-bound CD8+ αβ T lymphocytes and γδ T lymphocytes in the blood sample, wherein increased levels of activated, gut-bound CD8+ αβ T lymphocytes and γδ T lymphocytes compared to the levels of activated, gut-bound CD8+αβ T lymphocytes and γδ T lymphocytes in a control sample indicate that the subject has celiac disease. The “control” sample can be a blood sample obtained from a normal subject (e.g. an individual known to not have celiac disease or any condition or symptom associated with the disease) or a subject with inactive disease, such as a subject who has not consumed any gluten for a period long enough to allow the autoimmune response to decline (e.g., no gluten consumption for at least two weeks and preferably at least one month). Activated, gut-bound CD8+ αβ T lymphocytes and γδ T lymphocytes can be identified by detection of the activation marker, CD38, and the intestinal homing markers, CD103 and β7 integrin. Gluten can be ingested by the subject orally, for example, in the form of food (e.g., bread or wafer), a powder, or a pill in single or multiple doses over 1 to 3 days. In one embodiment, a blood sample is obtained from the subject up to 6 days after the subject consumes gluten.

In one embodiment, the levels of activated, gut-bound CD8+ αβ T lymphocytes and γδ T lymphocytes are compared in blood samples collected from a subject before and after consuming gluten. An initial blood sample is obtained from a subject who has not consumed any gluten for a period long enough to allow the autoimmune response to decline (e.g., no gluten consumption for at least two weeks and preferably at least one month) and a second blood sample is obtained from the subject after consuming gluten for 1 to 3 days. The levels of CD8+ αβ T lymphocytes and γδ T lymphocytes in the two blood samples are compared, wherein increased levels of CD8+ αβ T lymphocytes and γδ T lymphocytes in the second blood sample collected after gluten consumption indicate that the subject has celiac disease.

In certain embodiments, the method further comprises comparing the levels of activated, gut bound CD8+ αβ T lymphocytes and γδ T lymphocytes from the subject with reference levels for activated, gut bound CD8+ αβ T lymphocytes and γδ T lymphocytes. The reference levels can represent the levels of CD8+ αβ T lymphocytes and γδ T lymphocytes found in one or more samples of one or more subjects without celiac disease (i.e., normal control samples). Alternatively, the reference values can represent the levels of CD8+ αβ T lymphocytes and γδ T lymphocytes found in one or more samples of one or more subjects with celiac disease.

The number of CD8+ αβ T lymphocytes and γδ T lymphocytes in a blood sample can be determined by any suitable method, including visual counting of cells observed microscopically or automated methods of cell counting. For example, cells can be counted by using a flow cytometer, Coulter counter, CASY counter, hemocytometer, or microscopic imaging. In one embodiment, levels of CD8+ αβ T lymphocytes or γδ T lymphocytes are determined by staining cells obtained from a blood sample and counting cells of interest using fluorescence microscopy. In particular, cellular markers may be detected by methods such as, but not limited to immunofluorescent antibody assay (IFA), enzyme-linked immuno-culture assay (ELICA), flow cytometry, cytometry by time-of-flight (CyTOF), and magnetic cell sorting. The relative frequency of a cell type expressing one or more markers can be determined, for example, by fluorescence-activated cell sorting (FACS).

In certain embodiments, the method further comprises detecting an increase in the number of CD8+ αβ T lymphocytes or γδ T lymphocytes expressing one or more cellular markers selected from the group consisting of αE (CD103), β7 integrin, and CD38 compared to the levels of the T lymphocytes expressing the one or more cellular markers in a control sample. In certain embodiments, the method further comprises detecting one or more additional cellular markers. In one embodiment, one or more cellular markers for a CD8+ αβ T cell selected from the group consisting of CD38, CD45RO, CD27, CD28, CD62L, and CCR7 are detected. In one embodiment, the method comprises counting the number of CD8+ αβ T cells having a phenotype of CD38⁺, CD45RO⁺, CD27⁻, CD28^{low}, CD62L⁻, and CCR7^{low}, wherein an increase in the number of CD8+ T cells having this phenotype compared to a control sample indicates that the subject has celiac disease. In another embodiment, one or more cellular markers for a γδ T cell selected from the group consisting of CD45RO and CD27 are detected. In one embodiment, the method comprises counting the number of γδ T cells having a phenotype of CD45RO⁺ and CD27⁻ wherein an increase in the number of γδ T cells having this phenotype compared to a control sample indicates that the subject has celiac disease.

In certain embodiments, the method further comprises detecting activation of an αβ or γδ T cell. Activation of a T cell can be determined, for example, by detecting T cell proliferation, expression of a cell marker, or secretion of a cell product, such as a cytokine, Fas ligand, perforin, or a granzyme. The T cell response can be evaluated by performing an immunoassay, such as, but not limited to an enzyme-linked immunosorbent spot (ELISPOT) assay, a T cell proliferation assay, flow cytometry, or time-of-flight mass cytometry (CyTOF) to detect, for example, changes in T cell surface or intracellular activation markers. Secretion of a cell product, such as a secretory molecule including, but not limited to IFN-γ, TNF-α, TNF-β, IL-2, IL-3, Fas ligand, perforin, or a granzyme may be detected by an ELISPOT assay. Cell markers including, but not limited to αE (CD103), β7 integrin, CD38, CD45RO, CD27, CD28, CD62L, and CCR7 can be detected, for example, by flow cytometry or CyTOF. The secretory molecule or cell marker or combination of secretory molecules or cell markers chosen for detection depends on whether the T cell is a CD8+ αβ T cell or a γδ T cell.

In another embodiment, the invention includes a method for treating a subject suspected of having celiac disease the method comprising: a) obtaining a blood sample comprising CD8+ αβ T lymphocytes and γδ T lymphocytes from the subject after 1 to 3 consecutive days of gluten consumption by the subject; b) diagnosing celiac disease in the subject according to a method described herein; and c) treating the subject with a gluten-free diet if increased levels of CD8+ αβ T lymphocytes and γδ T lymphocytes in the blood sample from the subject compared to the levels of αβ T lymphocytes and γδ T lymphocytes in a control sample indicate that the subject has celiac disease. In one embodiment, the method further comprises measuring the levels of activated, gut-bound CD8+ αβ T lymphocytes and γδ T lymphocytes after treating the subject with a gluten-free diet and comparing to reference levels for gut-bound CD8+ αβ T lymphocytes and γδ T lymphocytes.

In another embodiment, the invention includes an assay comprising: a) measuring the levels of activated, gut-bound CD8+ αβ T lymphocytes and γδ T lymphocytes in a blood sample collected from a patient administered a gluten challenge for 1 to 3 days prior to collection of the blood sample; and b) comparing the levels of activated, gut-bound CD8+ αβ T lymphocytes and γδ T lymphocytes in the blood sample with reference levels for activated, gut-bound CD8+ αβ T lymphocytes and γδ T lymphocytes for subjects without celiac disease, wherein increased levels of activated, gut-bound CD8+ αβ T lymphocytes and γδ T lymphocytes compared to the reference levels indicate that the patient has celiac disease.

In certain embodiments, the assay further comprises detecting an increase in the number of CD8+ αβ T lymphocytes or γδ T lymphocytes expressing one or more cellular markers selected from the group consisting of αE (CD103), β7 integrin, and CD38 compared to the levels of the T lymphocytes expressing the one or more cellular markers in a control sample. In certain embodiments, the assay further comprises detecting one or more additional cellular markers. In one embodiment, one or more cellular markers for a CD8+ αβ T cell selected from the group consisting of CD38, CD45RO, CD27, CD28, CD62L, and CCR7 are detected. In one embodiment, the assay comprises counting the number of CD8+ αβ T cells having a phenotype of CD38⁺, CD45RO⁺, CD27⁻, CD28^{low}, CD62L⁻, and CCR7^{low}, wherein an increase in the number of CD8+ T cells having this phenotype compared to a control sample indicates that the patient has celiac disease. In another embodiment, one or more cellular markers for a γδ T cell selected from the group consisting of CD45RO and CD27 are detected. In one embodiment, the assay comprises counting the number of γδ T cells having a phenotype of CD45RO⁺ and CD27⁻, wherein an increase in the number of γδ T cells having this phenotype compared to a control sample indicates that the patient has celiac disease.

These and other embodiments of the subject invention will readily occur to those of skill in the art in view of the disclosure herein.

### BRIEF DESCRIPTION OF THE FIGURES

FIGS. 1A-1C show induction of activated, gut homing CD8⁺ αβ and γδ⁺ T cells in peripheral blood of celiac patients following oral gluten challenge. FIG. 1A shows a representative FACS analysis of total CD8⁺ αβ and γδ T cell (left) and CD4⁺ T cell (right) responses to oral gluten challenge in celiac disease versus a non-celiac control. Expansion of CD38⁺CD103⁺ and gluten tetramer⁺ CD4⁺ T cell populations is seen on day 6 following gluten challenge in celiac disease. FIG. 1B shows the relative frequency of αEβ7CD38⁺ CD8⁺ as a percentage of total CD8⁺ cells (left) and relative frequency of αEβ7CD38⁺ γδ cells as a percentage of total γδ T cells (right). FIG. 1C shows a time course experiment showing relative percentage of CD38⁺, CD103⁺ CD8⁺ (top), CD38⁺CD103⁺γδ⁺ (middle), and gluten tetramer⁺CD4⁺ (bottom) in the same patient at indicated time points following oral gluten challenge. Parallel recruitment of CD38⁺CD103⁺ and gluten tetramer⁺ cells peak on day 6 following gluten challenge before returning to baseline.

FIGS. 2A and 2B show that peripheral blood αEβ7⁺ CD38⁺ T cells induced by oral gluten challenge express surface markers of memory cells, and resemble intestinal epithelial lymphocytes from celiac mucosal biopsies. FIG. 2A shows a mass cytometry (CyTOF) analysis of total peripheral blood CD8⁺ (left) and total intestinal CD8⁺ cells with respect to CD103 and CD38 expression. CyTOF analysis of peripheral blood αEβ7⁺CD38⁺ CD8⁺ T cells (light gray) and total intestinal CD8⁺ T cells (dark gray) are overlaid upon total peripheral blood CD8⁺ T cells (medium gray). PB-IE and celiac intestinal CD8⁺ cells are predominantly CD38⁺CD45RO⁺CD45RA⁻ CD27⁻CD28^{low}CD62L⁻CCR7⁻, consistent with an effector memory phenotype. FIG. 2B shows a CyTOF analysis of total peripheral blood γδ and total intestinal γδ T cells with respect to CD103 and CD38 expression (top panels). CyTOF analysis of total peripheral blood γδ, αEβ7⁺CD38⁺ γδ and total celiac intestinal γδ with respect to CD27 and CD45RA expression (bottom panels). PB-IE and celiac intestinal γδ cells are predominantly CD27⁻ and CD45RA⁻, consistent with a memory phenotype.

FIGS. 3A and 3B show that αEβ7CD38⁺CD8⁺ T cells can produce IFNγ but do not express higher levels of perforin or NKG2D relative to total blood CD8⁺ T cells. FIG. 3A shows that stimulated αEβ7⁺CD38⁺CD8⁺ T cells but not αEβ7⁺CD38⁺ γδ T cells are able to produce TNFα and IFNγ in response to stimulation with PMA and ionomycin. FIG. 3B shows that αEβ7⁺CD38⁺CD8⁺ T cells do not express higher levels of perforin or NKG2D than total CD8⁺ T cells.

FIGS. 4A-4D show that single-cell TCR sequencing of peripheral blood αEβ7⁺CD38⁺ CD8⁺ and αEβ7⁺CD38⁺ γδ T cells reveals clonal expansion upon gluten challenge in celiac disease with identical clones reappearing upon repeat gluten challenge. FIG. 4A shows individual TCR clone counts upon gluten challenge. αEβ7⁺CD38⁺ CD8⁺ TCRs were sequenced in five separate patients following gluten challenge, two of whom underwent re-challenge. αEβ7⁺CD38⁺ γδ TCRs were sequenced in three patients, one of whom underwent re-challenge. Each individual dot represents a distinct TCR clone. Size of dots and position along the Y-axis, plotted on a log scale, indicates the relative frequency of a particular clone. Total number of clones found in each patient is indicated in parentheses. FIGS. 4B and 4C show that identical αEβ7⁺CD38⁺ CD8⁺ TCRβ clones are re-encountered upon repeat gluten challenge within the same patient. CDR3β motif and frequency are indicated. FIG. 4B lists TCRβ CDR3 sequences from patient 1 (SEQ ID NO:46, SEQ ID NO:72, SEQ ID NO:48, SEQ ID NO:52, SEQ ID NO:56, SEQ ID NO:58, SEQ ID NO:60, SEQ ID NO:54, SEQ ID NO:64, SEQ ID NO:99, SEQ ID NO:66, SEQ ID NO:102, SEQ ID NO:78, and SEQ ID NO:53). FIG. 4C lists TCRI3 CDR3 sequences from patient 2 (SEQ ID NO:145, SEQ ID NO:151, SEQ ID NO:153, SEQ ID NO:227, SEQ ID NO:243, SEQ ID NO:148, SEQ ID NO:174, SEQ ID NO:176, SEQ ID NO:192, and SEQ ID NO:210). FIG. 4D shows that identical αEβ7⁺CD38⁺ CD8⁺ TCRδ clones (SEQ ID NO:459, SEQ ID NO:460, SEQ ID NO:476, SEQ ID NO:462, SEQ ID NO:463, SEQ ID NO:480, SEQ ID NO:472, SEQ ID NO:475, and SEQ ID NO:479) are re-encountered upon repeat gluten challenge within the same patient. CDR3δ motif and frequency are indicated.

FIGS. 5A-5F show that convergent αEβ7⁺CD38⁺CD8⁺TCRβ and αEβ7⁺CD38⁺TCRδ CDR3 motifs are found among clones within the same celiac patient and across different patients following gluten challenge. FIG. 5A shows the relative frequency of TRBV gene usage in unique (non-redundant) TCR clones in celiac patients. Comparison to a reference database of sequences shows that TRBV7-9, TRBV7-8, and TRBV28 are overrepresented in celiac patients versus controls. FIG. 5B shows the relative frequency of TRBV7-9, TRBV7-8, and TRBV28 usage in unique TCR clones in individual celiac patient compared to controls. FIG. 5C shows that convergent motifs (SEQ ID NO:45 and SEQ ID NOS:666-668) seen in TCRβ clones utilizing TRBV7-9, TRBV7-8, and TRBV28 and in TCRδ clones utilizing TRDV1 are statistically significant. FIG. 5D shows that the convergent motif CxxxxGN (SEQ ID NO:666) is seen in TCRβ clones utilizing TRBV7-9. Frequency of each clone is indicated and total number of T cells sequenced in the patient is indicated in parenthesis. Protein sequences with corresponding DNA sequences are shown. Within the protein sequences, dark gray indicates absolutely conserved amino acids, while medium gray indicates conserved amino acids that are encoded within the V or J genes. Within the DNA sequences, nucleotides in black are formed through N or P addition, while nucleotides in light grey are encoded by D genes. Boxes around frequency numbers highlight distinct clones sharing identical protein sequences FIG. 5E shows that the convergent motif CxxxxGT (SEQ ID NO:667) is seen in TCRβ clones utilizing TRBV7-8. FIG. 5F shows that the convergent motif CxxxxxxxxYWGI (SEQ ID NO:45) is seen in TCRδ clones utilizing TRBV1.

FIG. 6 shows that the phenotype and functional capacity of αEβ7⁺CD38⁺ CD8⁺ T cells resembles effector memory cells and resembles CD8 T-IEL. Functional capacities of the indicated cell types with respect to the indicated markers are plotted as a heat plot. Cells were stimulated with PMA and ionomycin and analyzed for the indicated cell surface or intracellular markers. Cells are segregated based on stringent criteria: Naive (CD45RA⁺CD27⁺CD62L⁺CCR7⁺), Effector Memory (Tem, CD45RA⁻CD27⁻CD62L⁻CCR7⁻), Central Memory (Tcm, CD45RA⁻CD27⁺CD62L⁺CCR7⁺), Short Lived Effector (Tsle, CD45RA⁺CD27⁻CD62L⁻CD28⁻), Celiac PB (CD3⁺CD8⁺CD103⁺Integrinβ7⁺CD38⁺), and Celiac Biopsy (CD3⁺CD8⁺). All blood samples analyzed are from celiac patients on day 6 following gluten challenge. Biopsy samples are from different celiac patients with active celiac disease including villous blunting and IEL expansion by histologic examination.

### DETAILED DESCRIPTION

The practice of the present invention will employ, unless otherwise indicated, conventional methods of medicine, virology, chemistry, biochemistry, recombinant DNA techniques, and immunology, within the skill of the art. Such techniques are explained fully in the literature. See, e.g., Celiac Disease: Methods and Protocols (Methods in Molecular Medicine, M. N. Marsh ed., Humana Press; 1st edition, 2000; Frontiers in Celiac Disease (Pediatric and Adolescent Medicine, A. Fasano, R. Troncone, D. Branski eds., S Karger Pub; 1^{st} edition, 2008); T Cell Protocols (Methods in Molecular Biology, G. De Libero ed., Humana Press, 2^{nd} edition, 2008); The Immunoassay Handbook: Theory and Applications of Ligand Binding, ELISA and Related Techniques (D. G. Wild ed., Elsevier Science; 4^{th} edition, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a T cell” includes a mixture of two or more T cells, and the like.

The term “about”, particularly in reference to a given quantity, is meant to encompass deviations of plus or minus five percent.

As used herein, the terms “T cell immune response” or “T cell response” refer to activation of antigen-specific T cells as measured by cell proliferation or expression of molecules on their cell surface or secretion of proteins such as cytokines.

A “reference level” or “reference value” of CD8+αβ T or γδ T lymphocytes means a level of the lymphocytes that is indicative of a particular disease state, phenotype, or predisposition to developing a particular disease state or phenotype, or lack thereof. A “reference level” of CD8+αβ or γδ T lymphocytes may be an absolute or relative amount of the CD8+αβ or γδ T lymphocytes, a range of amount of the CD8+αβ or γδ T lymphocytes, a minimum and/or maximum amount of the CD8+αβ or γδ T lymphocytes, a mean amount of the CD8+αβ or γδ T lymphocytes, and/or a median amount of the CD8+αβ or γδ T lymphocytes; and, in addition, “reference levels” of combinations of CD8+αβ and γδ T lymphocytes may also be ratios of absolute or relative amounts of the two lymphocytes with respect to each other. Appropriate reference levels of the CD8+αβ and γδ T lymphocytes for a particular disease state, phenotype, or lack thereof may be determined by measuring levels of the CD8+αβ and γδ T lymphocytes in one or more appropriate subjects, and such reference levels may be tailored to specific populations of subjects (e.g., a reference level may be age-matched or gender-matched so that comparisons may be made between CD8+αβ or γδ T lymphocytes levels in samples from subjects of a certain age or gender and reference levels for a particular disease state, phenotype, or lack thereof in a certain age or gender group). Such reference levels may also be tailored to specific techniques that are used to measure levels of the CD8+αβ or γδ T lymphocytes in biological samples (e.g., flow cytometry, time-of-flight mass cytometry, immunoassays, etc.), where the levels of the T lymphocytes may differ based on the specific technique that is used.

A “similarity value” is a number that represents the degree of similarity between two things being compared. For example, a similarity value may be a number that indicates the overall similarity between a patient's T lymphocyte profile and reference levels for the T lymphocytes in one or more control samples or a reference T lymphocyte profile (e.g., the similarity to a celiac disease T lymphocyte profile or a normal control T lymphocyte profile). The similarity value may be expressed as a similarity metric, such as a correlation coefficient, or may simply be expressed as a difference in the number of T lymphocytes of a particular type, or the aggregate of differences in the numbers of more than one type of T lymphocyte in a patient sample and a control sample or reference T lymphocyte profile.

As used herein, the term “epitope” generally refers to the site on an antigen which is recognized by a T-cell receptor and/or an antibody. It can be a short peptide derived from a protein antigen. Several different epitopes may be carried by a single antigenic molecule.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition that elicits a cellular immune response may serve to sensitize a subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Methods of measuring cell-mediated immune response include measurement of intracellular cytokines or cytokine secretion by T-cell populations, or by measurement of epitope specific T-cells (e.g., by the tetramer technique) (reviewed by McMichael, A. J., and O'Callaghan, C. A., J. Exp. Med. 187(9)1367-1371, 1998; Mcheyzer-Williams, M. G., et al, Immunol. Rev. 150:5-21, 1996; Lalvani, A., et al, J. Exp. Med. 186:859-865, 1997).

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain FIT molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” to an antibody or TCR or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies or TCRs bind to a particular protein or peptide at least two times the background and do not substantially bind in a significant amount to other proteins or peptides present in the sample. Specific binding to an antibody or TCR under such conditions may require an antibody or TCR that is selected for its specificity for a particular protein or peptide. Typically a specific or selective reaction will be at least twice background signal or noise and more typically more than 10 to 100 times background.

The terms “label” and “detectable label” refer to a molecule capable of detection, including, but not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to radiolabels (e.g., ³H, ¹²⁵I, ³⁵S, ¹⁴C, or ³²P), stable (non-radioactive) heavy isotopes (e.g., ¹³C or ¹⁵N), phycoerythrin, Alexa dyes, fluorescein, 7-nitrobenzo-2-oxa-1,3-diazole (NBD), YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease. Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, such as, in the case of celiac disease, the levels of activated CD8+αβ T lymphocytes and γδ T lymphocytes (e.g., including lymphocytes with particular combinations of cellular markers), the presence, absence, or amount of which is indicative of the presence or absence of the disease, disorder or dysfunction.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that people who have celiac disease show increased numbers of activated, gut-bound CD8+ αβ T lymphocytes and γδ T lymphocytes in their peripheral blood after consuming gluten (see Example 1). In particular, the present invention relates to a diagnostic test for celiac disease based on the detection of elevated levels of CD8+αβ T lymphocytes and γδ T lymphocytes in the peripheral blood of a subject after as little as one to three days of gluten consumption. This diagnostic test has a number of advantages over current serological and endoscopic tests for celiac disease, including that the test is noninvasive, relatively inexpensive, and requires voluntary gluten ingestion by a subject over a shorter period of time.

In order to further an understanding of the invention, a more detailed discussion is provided below regarding methods of diagnosing celiac disease by analysis of CD8+αβ and γδ T lymphocytes.

In one aspect, the invention includes a method for diagnosing celiac disease in a subject, the method comprising: a) obtaining a blood sample comprising peripheral blood lymphocytes from the subject after the subject has consumed gluten for 1 to 3 days; and b) measuring the levels of activated, gut-bound CD8+ αβ T lymphocytes and γδ T lymphocytes in the blood sample, wherein increased levels of activated, gut-bound CD8+ αβ T lymphocytes and γδ T lymphocytes compared to the levels of activated, gut-bound CD8+ αβ T lymphocytes and γδ T lymphocytes in a control sample indicate that the subject has celiac disease. Activated, gut-bound CD8+ αβ T lymphocytes and γδ T lymphocytes can be identified by detection of the activation marker, CD38, and the intestinal homing markers, CD103 and β7 integrin. Gluten can be ingested by the subject orally, for example, in the form of food (e.g., bread or wafer), a powder, or a pill in single or multiple doses over 1 to 3 days. In one embodiment, a blood sample is obtained from the subject up to 6 days after the subject has consumed gluten.

Blood samples obtained from the subject to be diagnosed comprise peripheral blood lymphocytes, including CD8+αβ T lymphocytes and γδ T lymphocytes, and can be obtained from a subject by conventional techniques, such as by venipuncture. The levels of T lymphocytes from the subject are compared to a “control” sample, that is, a blood sample obtained from a normal subject (e.g. an individual known to not have celiac disease or any condition or symptom associated with the disease) or a subject with inactive disease, such as a subject who has not consumed any gluten for a period long enough to allow the autoimmune response to decline (e.g., no gluten consumption for at least two weeks and preferably at least one month).

In one embodiment, the invention includes a method of diagnosing celiac disease by comparing the levels of CD8+αβ T lymphocytes and γδ T lymphocytes in blood samples collected from a subject before and after consuming gluten. An initial blood sample is obtained from a subject who has not consumed any gluten for a period long enough to allow the autoimmune response to decline (e.g., no gluten consumption for at least two weeks and preferably at least one month). A second blood sample is obtained from the subject after consuming gluten for 1 to 3 days. The levels of CD8+αβ T lymphocytes and γδ T lymphocytes in the two blood samples are compared, wherein increased levels of CD8+αβ T lymphocytes and γδ T lymphocytes in the second blood sample collected after gluten consumption indicate that the subject has celiac disease.

In certain embodiments, the levels of CD8+αβ T lymphocytes and γδ T lymphocytes from the subject are measured and compared with reference levels for the CD8+αβ T lymphocytes and γδ T lymphocytes. When analyzing the levels of T lymphocytes in a blood sample, the reference value ranges used for comparison can represent the levels of CD8+αβ T lymphocytes and γδ T lymphocytes found in one or more samples of one or more subjects without celiac disease (i.e., normal control samples). Alternatively, the reference values can represent the levels of CD8+αβ T lymphocytes and γδ T lymphocytes found in one or more samples of one or more subjects with celiac disease.

The levels of the CD8+αβ T lymphocytes and γδ T lymphocytes in a blood sample can be determined by any suitable method known in the art, including visual counting of cells observed microscopically or automated methods of cell counting. For example, cells can be counted by using a flow cytometer, Coulter counter, CASY counter, hemocytometer, or microscopic imaging. Cells can be distinguished by their shape, intracellular structures, staining characteristics, and the presence of cell markers. In particular, cell markers can be detected using methods, including but not limited to immunofluorescent antibody assay (IFA), enzyme-linked immuno-culture assay (ELICA), flow cytometry, cytometry by time-of-flight (CyTOF), and magnetic cell sorting. See. e.g., Stewart et al. (2000) Methods Cell Sci. 22(1):67-78; Cunningham (2010) Methods Mol. Biol. 588:319-339; herein incorporated by reference.

For example, various visual counting methods can be used. A hemocytometer can be used to count cells viewed under a microscope. The hemocytometer contains a grid to allow manual counting of the number of cells in a certain area and a determination of the concentration of cells in a sample. Alternatively, cells can be plated on a petri dish containing a growth medium. The cells are plated at a dilution such that each cell gives rise to a single colony. The colonies can then be visually counted to determine the concentration of particular cells types that were present in a sample.

Automated cell counting can be performed with a flow cytometer, Coulter counter, CASY counter, or by automated microscopic imaging analysis. Coulter and CASY counters can be used to measure the volumes and numbers of cells. Flow cytometry can be used for automated cell counting and sorting and for detecting surface and intracellular markers. Additionally, microscopic analysis of cells can be automated. For example, microscopy images can be analyzed using statistical classification algorithms that automate cell detection and counting. See, e.g., Shapiro (2004) Cytometry A 58(1):13-20; Glory et al. (2007) Cell Mol. Biol. 53(2):44-50; Han et al. (2012) Machine Vision and Applications 23 (1): 15-24; herein incorporated by reference.

In particular, flow cytometry can be used to distinguish subpopulations of cells expressing different cellular markers and to determine their frequency in a population of cells (e.g., frequency of αEβ7CD38⁺ CD8⁺ T cells in total population of CD8+ T cells or frequency of αEβ7CD38+γδ cells in total population of γδ T cells). Typically, whole cells are incubated with antibodies that specifically bind to the cellular markers. The antibodies can be labeled, for example, with a fluorophore, isotope, or quantum dot to facilitate detection of the cellular markers. The cells are then suspended in a stream of fluid and passed through an electronic detection apparatus. In addition, fluorescence-activated cell sorting (FACS) can be used to sort a heterogeneous mixture of cells into separate containers. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition,1990; Flow Cytometry: Principles and Applications, (ed. Macey), Humana Press 1^{st} edition, 2007; herein incorporated by reference in their entireties.)

Cytometry by time-of-flight (CyTOF), also known as mass cytometry, is another method that can be used for detection of cellular markers in whole cells. CyTOF uses transition element isotopes as labels for antibodies, which are detected by a time-of-flight mass spectrometer. Unlike conventional flow cytometry, CyTOF is destructive to cells, but has the advantage that it can be used to analyze more cell markers simultaneously. CyTOF can be used in the methods of the invention to identify cell markers, including, but not limited to αE (CD103), β7 integrin, CD38, CD45RO, CD27, CD28, CD62L, CCR7, and CD57. See, e.g., Bendall et al. (2012) Trends in Immunology 33:323-332; Newell et al. (2012) Immunity 36(1):142-52; Ornatsky et al. (2010) J. Immunol. Methods 361 (1-2):1-20; Bandura et al. (2009) Analytical Chemistry 81:6813-6822; Chen et al. (2012) Cell Mol. Immunol. 9(4):322-323; and Cheung et al. (2011) Nat. Rev. Rheumatol. 7(9):502-3; herein incorporated by reference in their entireties.)

The CD8+αβ T lymphocytes and γδ T lymphocytes can be further analyzed for activation. Any known method for evaluating T cell activation can be used to monitor the T cell response to gluten consumption. Activation of T cells has an induction phase in which T cells proliferate and differentiate and an effector phase, in which T cells carryout their functions. Therefore, T cells that have been activated in response to gluten consumption can be detected by cell proliferation assays or assays of their effector function, such as assays detecting expression of molecules on their cell surface or secretion of cytokines, granzymes, or perforin, or the ability of a CD8+ T cell to kill target cells.

For example, T cell activation may be detected with a cell proliferation assay. Proliferating cells are commonly detected using radioactive thymidine incorporation. Increased DNA synthesis in proliferating cells results in uptake of the radioactive thymidine and the amount of radioactive thymidine used by cells is correlated with the level of cellular proliferation. Cells undergoing proliferation are also more metabolically active, which can be detected based on their increased level of dehydrogenase activity. The levels of NADH and NADPH can be measured by their ability to reduce yellow colored 3-(4,5-dimethylthiazolyl-2)-2,5-diphenyltetrazolium bromide (MTT) to intracellular purple formazan. The resulting purple products can be solubilized and quantified spectrophotometrically. Alternatively, proliferating cells can be labeled with a fluorescent nucleic acid dye and detected by flow cytometry. See, e.g., Kruisbeek et al. (2004) Proliferative assays for T cell function. Curr. Protoc. Immunol., Chapter 3:Unit 3.12; Fulcher et al. (1999) Immunol. Cell Biol. 77(6):559-564; herein incorporated by reference in their entireties.

In another example, secretion of cytokines, granzymes, or perforin, or any other secretory molecule of interest by a T cell in response to activation may be detected by an enzyme-linked immunosorbent spot (ELISPOT) assay. For example, one or more of IFN-γ, TNF-α, TNF-β, IL-2, IL-3, Fas ligand, perforin, or a granzyme may be detected to determine if a CD8+ T cell is activated. Antibodies specific for a T cell secretory molecule are immobilized on a polyvinylidene fluoride (PVDF) membrane coating a microplate well. Next, T cells, antigen, and antigen presenting cells are added to the well. The cell product of interest secreted by activated T cells is captured locally by the immobilized antibody in the well. The captured secretory molecule can then be detected, for example, with a labeled antibody that recognizes an epitope of the captured secretory molecule. Typically, ELISPOT assays are performed with a biotinylated antibody which binds specifically to the captured secretory molecule. The biotinylated antibody can then be detected with an avidin-conjugated enzyme, such as avidin-horseradish peroxidase or avidin-alkaline phosphatase using a substrate that produces a colored enzyme product. The Fluorospot assay is a variation of the ELISPOT assay that instead uses multiple fluorescently labeled antibodies against secretory molecules for detection of T cell activation. See, e.g., Czerkinsky et al. (1983) J. Immunol. Methods 65 (1-2): 109-121; Augustine et al. (2012) Clin. Chim. Acta. 413(17-18):1359-1363; Anthony et al. (2012) Cells 1(2):127-140; Ahlborg et al. (2012) Methods Mol. Biol. 792:77-85; Rebhahn et al. (2008) Comput. Methods Programs Biomed. 92(1):54-65; herein incorporated by reference in their entireties.

In another example, analysis of intracellular or cell surface markers can be used to detect activated T cells. For example, flow cytometry or CyTOF can be used to detect expression of CD38 by activated CD8+αβ T cells and γδ T cells, natural killer (NK) receptors (e.g., NKG2D) by activated CD8+αβ T cells, or CD45RO or CD27 by activated γδ T cells.

In particular, the methods described herein can be used to determine an appropriate treatment for a subject suspected of having celiac disease. In one embodiment, the invention includes a method for treating a subject suspected of having celiac disease, the method comprising: a) obtaining a blood sample comprising CD8+αβ T lymphocytes and γδ T lymphocytes from the subject after 1 to 3 consecutive days of gluten consumption by the subject; b) diagnosing celiac disease in the subject according to a method described herein; and c) treating the subject with a gluten-free diet if increased levels of CD8+αβ T lymphocytes and γδ T lymphocytes in the blood sample from the subject compared to the levels of αβ T lymphocytes and γδ T lymphocytes in a control sample indicate that the subject has celiac disease. In one embodiment, the method further comprises measuring the levels of activated, gut-bound CD8+αβ T lymphocytes and γδ T lymphocytes after treating the subject with a gluten-free diet and comparing to reference levels for gut-bound CD8+αβ T lymphocytes and γδ T lymphocytes.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Dietary Gluten Triggers Parallel Activation of CD4+ and CD8+αβ and γδ T Cells in Celiac Disease

Here, we report a massive gluten-dependent induction in the numbers of activated, gut-bound CD8⁺ TCR αβ⁺ T cells and TCR γδ⁺ T cells in the peripheral blood of patients with celiac disease. TCR sequencing analysis shows a high degree of clonal expansion and conserved TCR motifs, suggesting antigen-driven activation. Our results demonstrate a T cell cascade through which both CD8⁺ TCR αβ⁺ and TCR γδ⁺ T cells are purposefully activated and recruited to the gut in parallel with gluten-reactive CD4⁺ T cells in celiac disease. This T cell cascade may be relevant in other autoimmune diseases.

Methods

Gluten Challenge

Volunteers underwent oral gluten challenge as described (Brottveit et al. (2011) Am. J. Gastroenterol. 106:1318-1324; herein incorporated by reference). At the time of the participation, all volunteers maintained a gluten-free diet for at least one month. After the initial blood draw, volunteers consumed four slices of white bread per day for three consecutive days (days 1, 2 and 3) and returned for second blood draw on day 6. All celiac patient volunteers had a clinical diagnosis of celiac disease established by small intestinal biopsy in addition to serologic antibody testing. Healthy HLA-DQ2⁺ volunteers were either parents of children with celiac disease or individuals who endorsed gluten-intolerance. All healthy volunteers had a negative clinical diagnostic workup for celiac disease, and were able to comply with a gluten-free diet for at least one month prior to participation. Subjects were tested for HLA-DQ2 by PCR using the following primers:

| | |
|---|---|
| DQA5′- TCTTATGGTGTAAACTTGTACCAGTC | (SEQ ID NO: 1) |
| DQA3′- TCTTATGGTGTAAACTTGTACCAGTC | (SEQ ID NO: 2) |
| DQB5′- GCGTGCGTCTTGTGAGCAGAAG | (SEQ ID NO: 3) |
| DQB3′- CCTGTCCACCGCCGCCCGTTT | (SEQ ID NO: 4) |

All human sample collection and analysis were conducted with Stanford University IRB oversight.

Tetramer Analysis and Flow Cytometry

All FACS experiments were performed on ARIA II or LSRII instruments (Becton Dickinson) in the Stanford Shared FACS Facility. Water-soluble MHC-DQ2 molecules with covalently tethered peptides were produced in a baculovirus expression system as described (Quarsten et al. (2001) J. Immunol. 167:4861-4868; herein incorporated by reference). Two different MHC-DQ2 molecules with engineered biotinylation sites were produced with tethered diamidated T cell epitopes of α-gliadin, including the DQ2-α-I epitope (QLQPFPQPELPY, SEQ ID NO:5) and the DQ2-α-II epitope (PQPELPYPQPE, SEQ ID NO:6). Proteins were biotinylated, purified and stored in PBS with 50% glycerol at −20° C. Tetramers were prepared by incubating protein with streptavidin-fluorophore conjugates (ebiosciences) at a 4:1 molar ratio. Tetramer staining was performed at room temperature for 1 hour using 10 mg/ml of tetramer. The following antibody clones were used for flow cytometry: anti-CD3 (SK7, Biolegend), anti-CD4 (RPA-T4, Biolegend), anti-CD8 (OKT8, ebiosciences), anti-γδTCR (MHGD04, Invitrogen), anti-CD38 (HIT2, Biolegend), anti-integrinβ7 (FIB504, eBioscience), anti-CD103 (Ber-ACT8, Biolegend), CD27 (O323, eBioscience), anti-NKG2D (1D11, Biolegend). Dead cells were excluded using a LIVE/DEAD Fixable Dead Cell Stain kit (Invitrogen).

Intestinal Biopsy Preparation

Small intestinal biopsies were obtained with informed consent from celiac patients undergoing gastrointestinal endoscopy at Stanford University Hospital. 3-4 intestinal biopsy fragments were processed as described (Shacklett et al. (2009) Methods Mol. Biol. 485:347-356). In brief, biopsies were incubated in RPMI with 5% FCS containing 0.5 mg/ml of Type 4 collagenase (Worthington Biochemical). Cells were periodically disrupted during incubation by passing through a syringe topped with a blunt-ended 16 gauge needle. Lymphocytes were enriched through Percoll (GE Healthcare) gradient centrifugation. Time of Flight Mass Cytometry Staining was performed immediately afterwards on freshly isolated lymphocytes.

Time of Flight Mass Cytometry Staining and Data Acquisition

Time of Flight Mass Cytometry (CyTOF) Staining and data acquisition was performed as described (Newell et al. (2012) Immunity 36(1):142-152; herein incorporated by reference in its entirety). All antibody clones used for CyTOF can be found in Newell et al., supra. Cyropreserved PBMCs (or freshly isolated intestinal lymphocytes) were thawed and washed with complete RPMI before overnight recovery at 37° C. Cells were transferred to 96 well plates (or tubes), washed and resuspended in cytometry buffer (PBS, 0.05% sodium azide, 2 mM EDTA, and 2% fetal calf serum) for staining as previously described (Newell et al., supra). For stimulation, all cells were cultured for 3 hours at approximately 15×10⁶/ml in complete RPMI (10% fetal calf serum) plus 1× brefeldin A (eBioscience), 1× monensin (eBioscience), 2.5 μg/ml anti-CD107a, 1.25 μg/ml anti-CD107b, and 10 μM TAPI-2 (VWR International). For PMA+ionomycin stimulation, 150 ng/ml PMA+1 μM ionomycin were added to the cells.

At the end of the 3 hour stimulation, cells were pipetted vigorously to remove adherent cells from the plate and transferred to 96-well plates (or tubes), washed, and resuspended in cytometry buffer (PBS, 0.05% sodium azide, 2 mM EDTA, and 2% fetal calf serum). The cells were incubated for 30 minutes on ice with a prepared cocktail of metal-conjugated surface-marker antibodies at concentrations found to be effective in prior antibody tests. After surface staining, cells were washed 1× and resuspended in 20 μM indium-115-loaded maleimido-mono-amine-DOTA in PBS (a sulfhydryl reactive trivalent cation chelating bifunctional ligand, Macrocyclics #B-272, mixed with 0.5 molar ratio of 115-indium chloride and stock solution dissolved in DVS “L-buffer” [DVS Sciences] at 1 mM, stable at 4° C. and working much like commercially available amine-reactive Live/Dead staining reagents, Invitrogen). After 30 minutes on ice, the cells were washed 3 times in cytometry buffer and resuspended in PBS containing 2% paraformade (Electron Microscopy Sciences). After overnight fixation at 4° C., the cells were washed 2 times in 1× intracellular staining permeabilization buffer (eBioscience, Cat. 00-8333-56) and stained with a cocktail of intracellular antibodies on ice for 45 minutes, washed 2 times in cytometry buffer, and labeled for 20 minutes at room temperature with 250 nM iridium interchelator (DVS Sciences) suspended in PBS containing 2% paraformaldehyde.

Finally, the cells were washed 2 times in cytometry buffer, 2 times in PBS, and 2 times in distilled water before diluting to the appropriate concentration to achieve an acquisition rate<500 events/second on the CyTOF instrument. CyTOF data were acquired and analyzed on the fly, using dual-count mode (calibrated on the fly, combining pulse-count and intensity information) with noise-reduction mode turned off. All other settings were either default settings or optimized with tuning solution as instructed by DVS sciences. For cells that had undetectable levels of a given isotope (a zero value for a given parameter), the default setting on the software assigns these cells a random value between 0 and −1, creating a square distribution between 0 and −1.

Time of Flight Mass Cytometry Antibody Labeling

Purified antibodies (lacking carrier proteins) were purchased from the companies listed (Newell et al., supra). The antibodies were labeled 100 μg at a time according to instructions provided by DVS Sciences with heavy metal-preloaded maleimide-coupled MAXPAR chelating polymers via the “Pre-Load Method v1.1.”

Single-Cell Sorting and TCR Sequencing

Single-cell sorting was performed using an ARIA II cell sorter (Becton Dickinson). TCR sequences from single cells were obtained by a series of three nested PCR reactions performed as described (Su et al. (2013) Immunity 38:373-383; herein incorporated by reference in its entirety). For the first reaction, reverse transcription and preamplification were performed with a One-Step qRT-PCR kit according to the manufacturer's instructions (Qiagen) using multiplex PCR with multiple Vβ or Vδ region primers and a Cβ or Cδ region primer. When necessary, base degeneracy was incorporated into the primers to account for TCR polymorphism and ensure amplification of all known functional TCRVβ or TCRVδ and TCRCβ and Cδ regions identified in the IMGT database (imgt.org/). Next, an aliquot of the first reaction was used as a template for second PCR reaction using a set of multiple internally nested TCRVβ or TCRVδ primers and an internally nested Cβ or Cδ primers with HotStarTaq DNA polymerase kit (Qiagen). The second set of TCRV region primers also incorporated base degeneracy when needed and contained a common 23 base sequence at the 5⁺ end to enable further amplification with a common 23 base primer. The third and final PCR reaction was performed on an aliquot of the second reaction using a primer containing the common 23 base sequence (incorporated into the second set of Vβ primers) and a third internally nested Cβ or Cδ primer using Hotstar DNA polymerase (Qiagen). Amplified PCR products were treated with ExoSAP-IT (Affymetrix) and sequenced using primers from the final PCR reaction. TCR junctional region analysis was performed using IMGT/V-Quest (imgt.org/IMGT_vquest). Primer sequences for TCRβ sequencing can be found in Su et al., supra. Primer sequences for TCRδ can be found in Table 4.

TCR Sequencing Analysis

TCR sequence analysis was performed with VDJFasta (Glanville et al. (2011) Proc. Natl. Acad. Sci. USA 108:20066-20071). Segment classification was performed to reference segment databases from IMGT. CDR3 from all domains were extracted and translated using TCR-specific profile Hidden Markov Models, constructed from 95% non-redundant concatenations of IMGT V, D and J segments as described previously. A dataset of 165,291 naive CD8⁺ TCRβ sequences (Warren, R. L. et al. (2011) Genome Research 21:790-797) was used as a control for CDR3β convergence. To generate TCRδ reference sequences, between 10⁵ and 10⁶ TCRγδ⁺ T cells from the peripheral blood of eight different individuals and an IEL from one individual were sorted by flow cytometry. RNA was extracted using an RNeasy RNA extraction kit (Qiagen). RNA from each of these samples was amplified and sequenced using the primers described above. 18,579 total unique TCRδ sequences utilizing TRDV1 were used as a control for TCRδ convergence. Motif enrichment was evaluated by comparing the observed versus expected frequency of 2-mer and 3-mer motifs within CDR3β or CDR3δ clones utilizing the same V region. Enrichment was represented as the odds of encountering enrichment of the motif in the reference dataset to the degree observed in the selected set. The significance of motif enrichment was evaluated by using the Fisher's Exact test with the Bonferroni correction such that that P values<0.05/<howmany>=xe-x were considered to be statistically significant. Analysis was performed in R version 2.11.1. The most statistically significant examples are illustrated.

Results

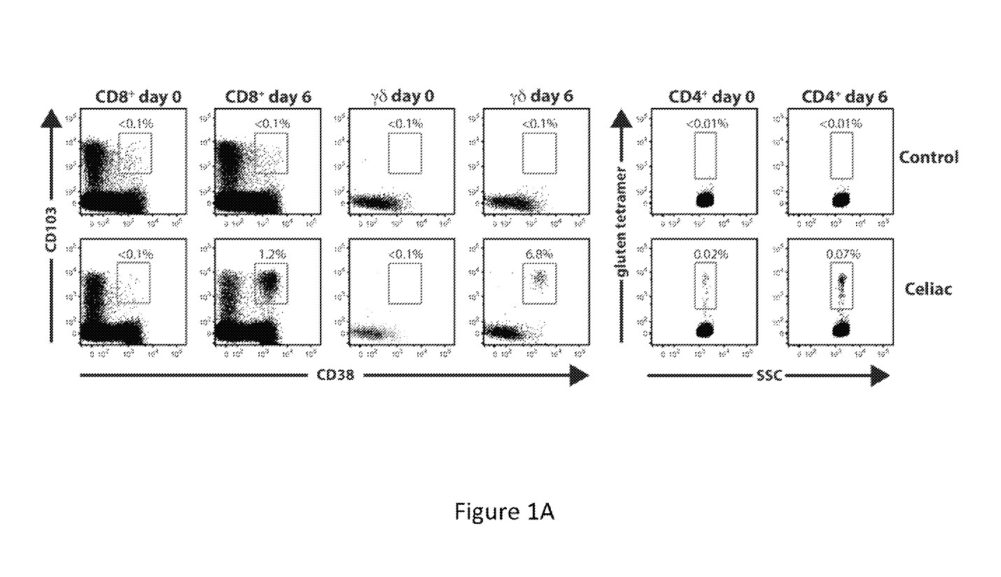

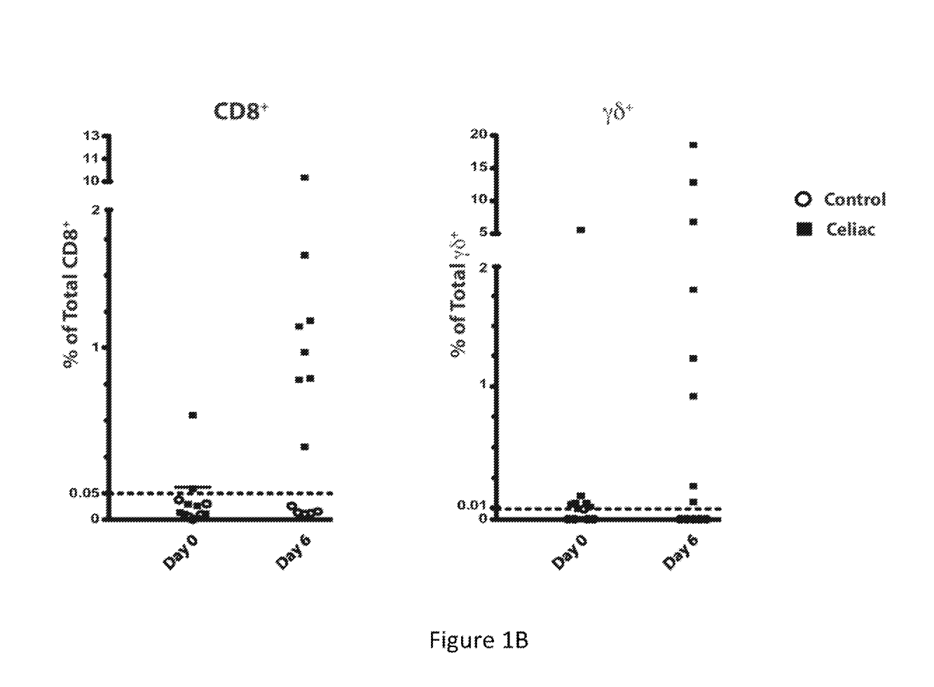

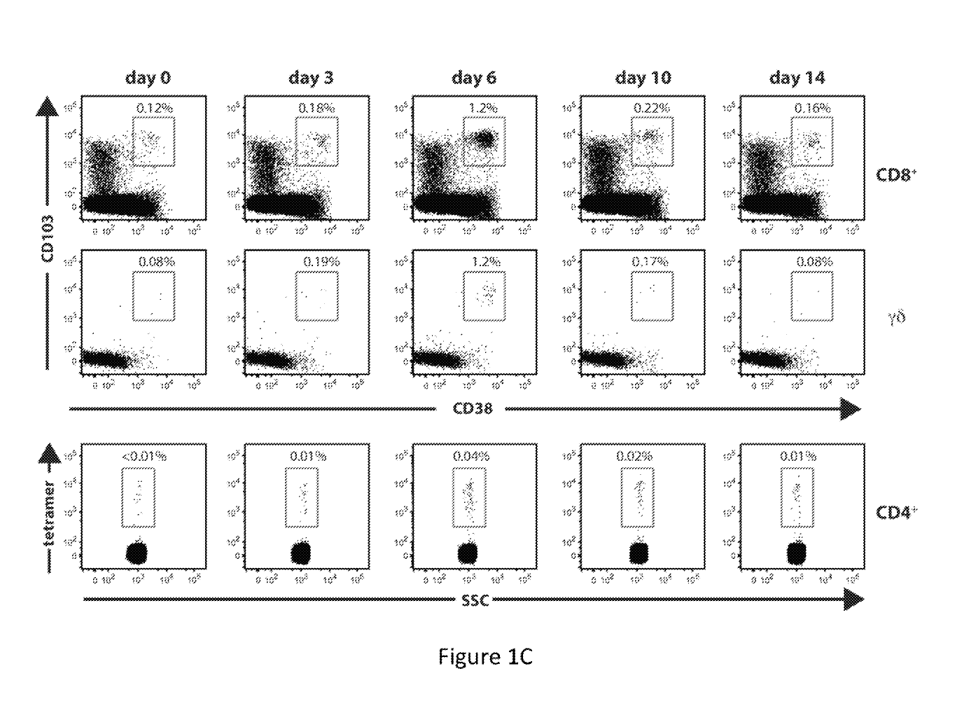

Gluten-specific CD4⁺ T cells are detectable in large numbers in the peripheral blood of celiac patients on a GFD six days following oral gluten challenge (Brottveit et al. (2011) Am. J. Gastroenterol. 106:1318-1324; Raki et al. (2007) Proc. Natl. Acad. Sci. USA 104:2831-2836). This phenomenon likely represents the initiation of a memory immune response to gluten, and captures gluten-reactive CD4⁺ T cells en route from mesenteric lymph nodes or gut-associated lymphoid tissue to the intestine. Strikingly, we also observed a large increase in the number of peripheral blood CD8⁺TCRαβ⁺ and TCRγδ⁺ T cells expressing the intestinal epithelial homing markers αE (CD103) and β7 integrins (Gorfu et al. (2009) Curr. Mol. Med. 9:836-850) and the activation marker CD38 in patients with celiac disease but not in HLA-DQ2⁺ controls who underwent oral gluten challenge after at least one month on a GFD (FIGS. 1A, 1B, and Table 1). We will henceforth refer to these CD38⁺ αEβ7⁺ peripheral blood intraepithelial-homing T cells as PB-IE CD8 and PB-IE GD. The number of peripheral blood αEβ7⁺ CD38⁺ T cells at day 6 following gluten challenge was profound, comprising on average 1.1% and 1.5% of total CD8⁺ and GD T cells, respectively (FIG. 1B, Table 1). A time course shows that the presence of PB-IE cells exactly parallels the presence of gluten-reactive CD4⁺ T cells and peaks at day 6 following gluten challenge (FIG. 1C). As reported for the peripheral blood gluten-reactive CD4⁺ T cell response to gluten challenge, the extent of the PB-IE CD8 and PB-IE GD response was highly variable between celiac patients, ranging from 0.37% to 10.17% of total peripheral blood CD8⁺αβ and 0.06% to 18.61% of total peripheral blood γδ T cells (Table 1). At least one celiac patient (celiac 2) had PB-IE CD8 and GD cells above background levels at day 0, but clearly showed a further increase following gluten challenge. The response was detectable in two celiac patients who underwent re-challenge after returning to a GFD for at least one month. An increase in gluten tetramer-positive CD4⁺ T cells was detected in the peripheral blood in all 5 HLA-DQ2⁺ celiac patients (not shown). The individual with the lowest detectable PB-IE CD8 and GD response (celiac 6) was an HLA-DQ8⁺ celiac patient whose disease was diagnosed incidentally by a positive biopsy but had equivocal antibody test results and no symptoms to dietary gluten. As has been described with gluten-specific CD4⁺ T cells, a significant amount of PB-IE cells were not present in the peripheral blood of people with active celiac disease (not shown). Three individuals with active celiac disease as determined by ongoing symptoms and positive auto-antibody titers were found to have PB-IE CD8 and PB-IE GD cell proportion below background levels of 0.05% and 0.01%, respectively. In summary, in individuals on a GFD who are challenged with gluten, all six celiac patients tested, but none of the five healthy HLA-DQ2⁺ controls, exhibited a clear increase in these cell populations at day 6 following gluten challenge (Table 1).

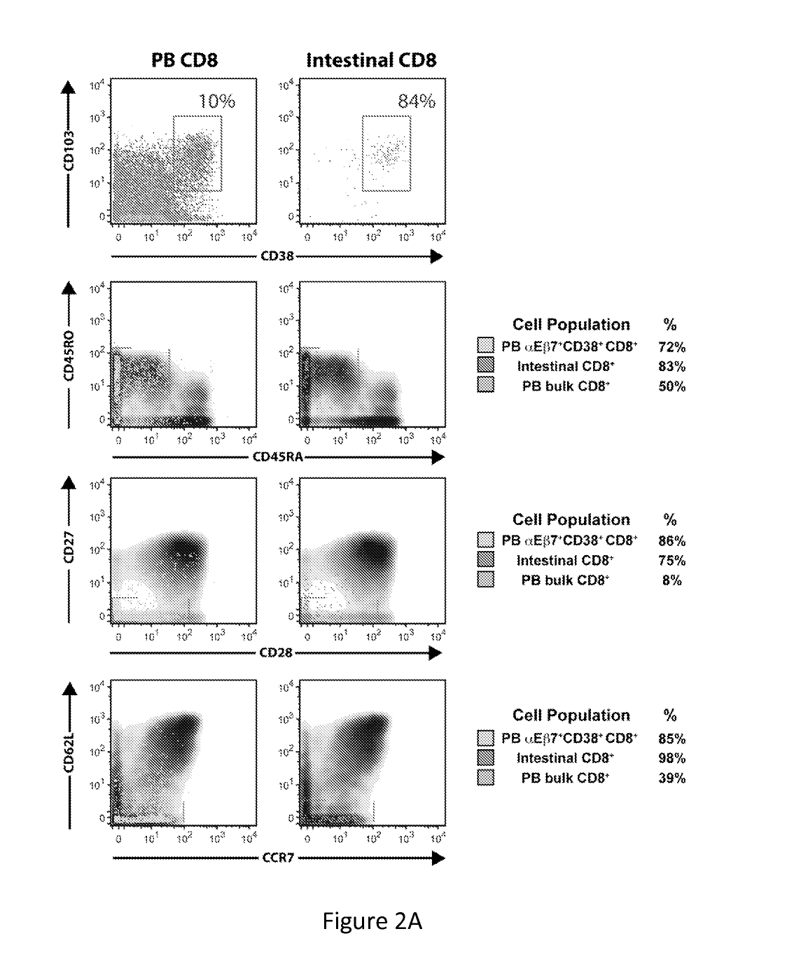

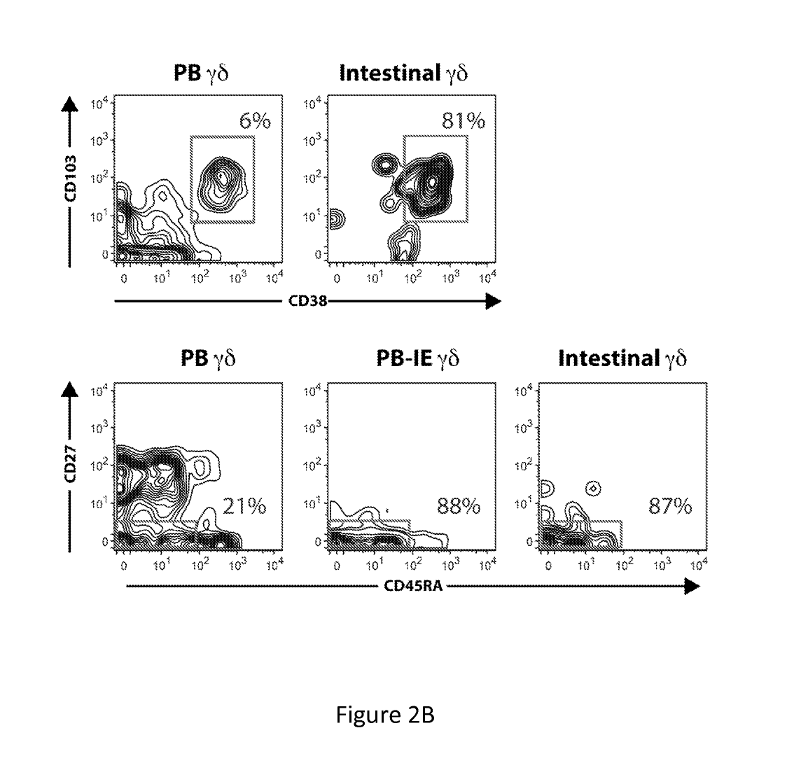

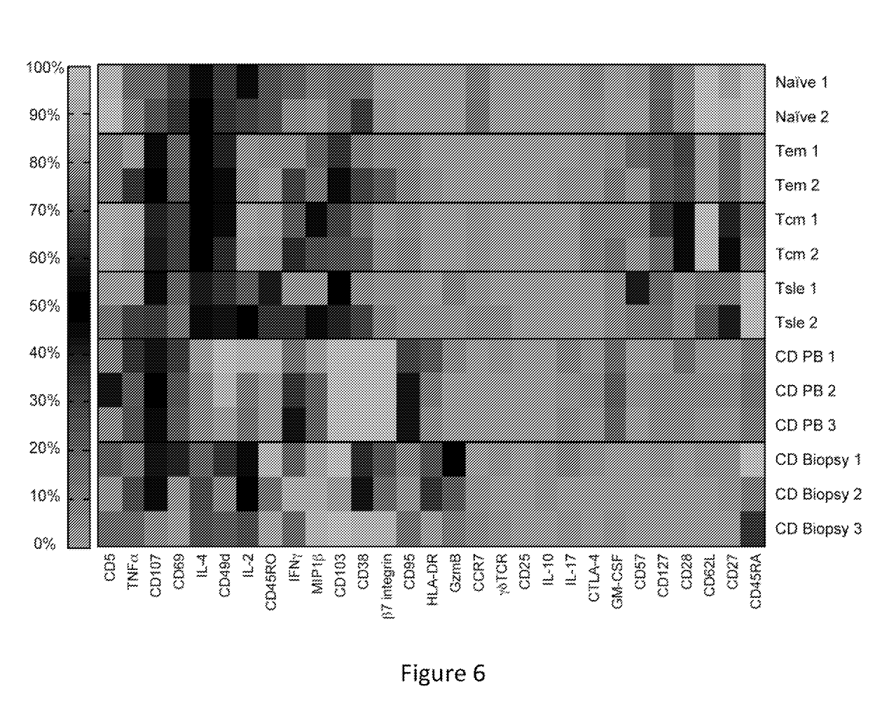

To investigate the phenotype and functional capacity of these induced PB-IE cell populations, we performed analysis by time-of-flight mass cytometry (CyTOF) (Newell et al. (2012) Immunity 36:142-152). Most PB-IE CD8 cells had the phenotype CD38⁺, CD45RO⁺, CD27⁻, CD28^{low}, CD62L⁻, CCR7^{low}(FIG. 2). Notably, the phenotype of PB-IE CD8 cells closely resembles the phenotype of CD8⁺ T cells isolated from duodenal tissue biopsy specimens of patients with active celiac disease (FIG. 2, FIG. 6). CD8⁺ cells of this phenotype have been reported to represent differentiated effectors, which tend to be short-lived and have greater effector potential (Newell, supra; Sallusto et al. (1999) Nature 401:708-712; Appay et al. (2002) Nat Med 8:379-385). These findings indicate that these PB-IE CD8 cells likely populate the intestinal epithelium in CD, and confirm that they are differentiated as effector cells prior to their arrival to the intestine. PB-IE GD cells are predominantly CD45RO⁺ and CD27⁻, mirroring intestinal GD cells from celiac biopsies (FIG. 2). Although γδ T cells are less well phenotypically characterized than αβ T cells, CD45RO⁺, CD27⁻γδ T cells have been characterized as memory phenotype cells (De Rosa et al. (2004) J. Immunol. 172:1637-1645).

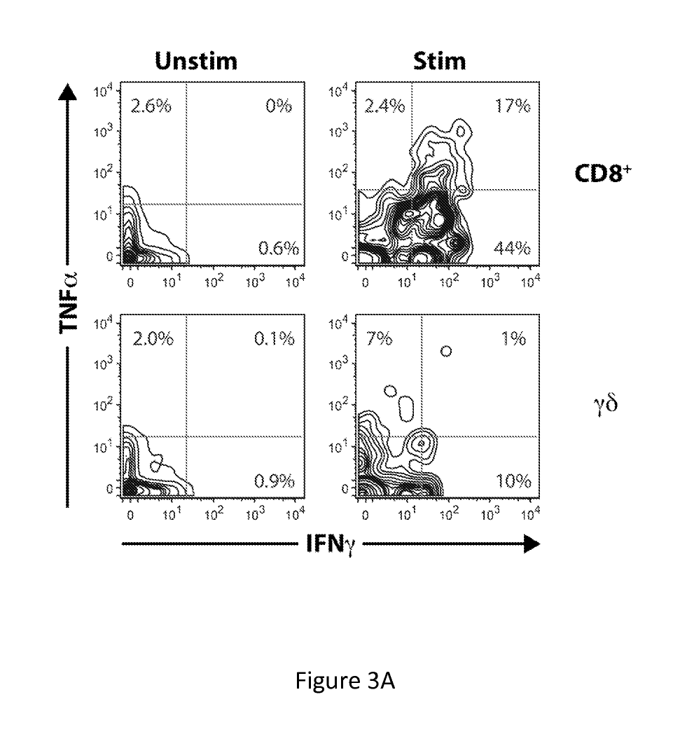

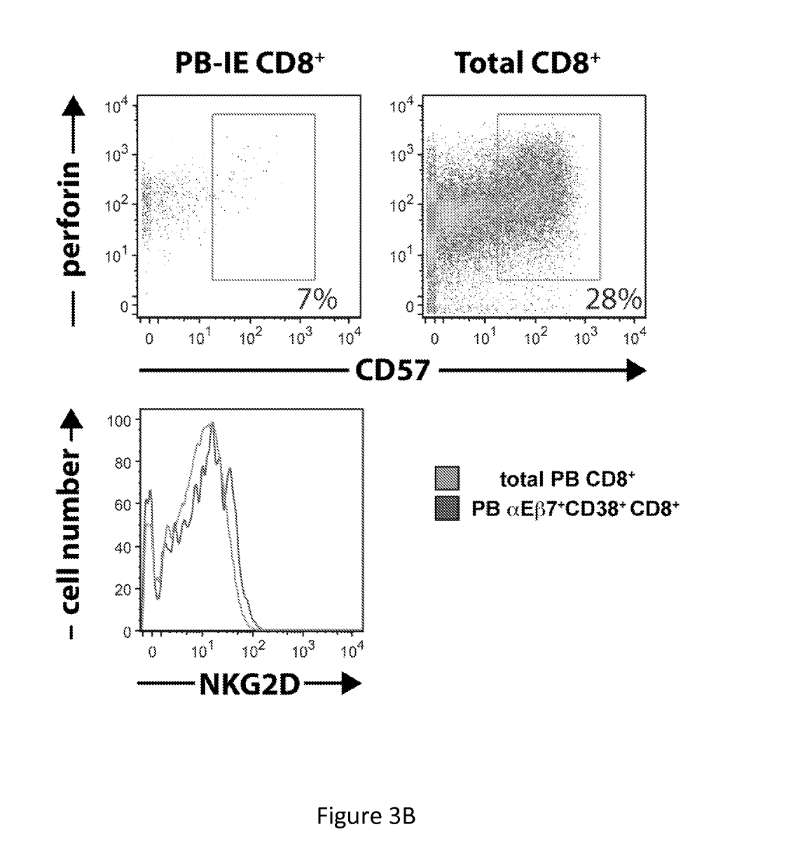

CD8 T-IEL are thought to be responsible for epithelial damage in CD. In active CD, CD8 T-IEL undergo significant expansion associated with IFN-γ expression (Olaussen et al. (2002) Scand. J. Immunol. 56:652-664). They also mediate cytotoxicity through perforin, granzymes and expression of NK receptors, including NKG2D (Meresse et al. (2004) Immunity 21:357-366; Di Sabatino et al. (2006) Gut 55:469-477; Meresse et al. (2006) J. Exp. Med. 203:1343-1355). GD T-IEL are increased in all stages of CD, but in contrast to CD8 T-IEL, their function is unclear. To further characterize the functional capacity of these PB-IE CD8 and GD cells, we performed intracellular IFN-γ and TNF-α analysis in response to stimulation with PMA and ionomycin. A significant proportion (>50%) of PB-IE CD8 cells is able to secrete IFN-γ in response to stimulation. In contrast, the PB-IE GD cells are largely inert with respect to cytokine secretion in response to stimulation (FIG. 3).

CD57 and intracellular perforin expression can delineate CD8⁺ cells with strong cytolytic function (Chattopadhyay et al. (2009) J. Leukoc. Biol. 85:88-97), and this function has been ascribed to CD8⁺ IEL cells in CD. We find that only a small proportion of PB-IE CD8 cells express CD57 and high levels of perforin (perforin^{hi}). Furthermore, the proportion of CD57⁺ perforin^{hi} PB-IE CD8 cells is less than that of total peripheral blood CD8⁺ αβ T cells in the same patient (FIG. 3). NKG2D expression has been reported to be upregulated in CD8 T-IEL, which have been described to develop NK-like properties and kill in a TCR-independent manner in CD (Meresse et al. (2004) Immunity 21:357-366; Meresse et al. (2006) J. Exp. Med. 203:1343-1355). We assessed NKG2D expression on the PB-IE CD8 cells, and could not find any appreciable difference in NKG2D expression on PB-IE CD8 cells compared to total peripheral blood CD8⁺ αβ T cells in celiac patients following gluten challenge. These data show that while surface marker analysis indicates that PB-IE cells have an effector phenotype and PB-IE CD8 cells have the capacity to secrete IFN-γ in response to stimulation, they largely do not express perforin, CD57 or higher levels of NKG2D. These observations suggest that CD8 T-IEL may rely on tissue-derived factors for full functional capacity. Despite these differences, functional and phenotypic analysis of PB-IE cells shows that PB-IE CD8 cells closely resemble peripheral blood effector memory cells and CD8 cells from intestinal biopsy (FIG. 6).

TCR sequence analysis within certain populations can ascertain whether a particular T cell population is expanded and possibly stimulated by antigen. Single-cell TCR sequencing enables a non-biased means to assess TCR repertoire within small populations of T cells without the need to expand T cell clones in culture (Su et al. (2013) Immunity 38:373-383). To validate our technique, we sorted and sequenced 90 single DQ2-a-II tetramer-reactive T cells from the blood of two individuals with celiac disease at day 6 following oral gluten challenge (Table 2). Sequences were successfully obtained from 77/90 (86%) of wells into which single T cells were sorted. No sequences were obtained from control wells into which no cells were sorted. The sequences we obtained of DQ2-a-II tetramer-reactive T cells were compared with published sequences of TCRV7-2⁺ DQ2-a-II reactive T cells from blood and tissue (Qiao et al. (2011) J. Immunol. 187:3064-3071). Consistent with this published report, the majority of our TCRβ sequences utilized TRBV7-2 and we found the same dominant conserved Arginine in position 5 of the CDR3β loop (Table 2). In addition, we were also able to successfully sequence multiple other TCRVIβ genes by using multiplex PCR rather than the single TCRV7-2 primer.

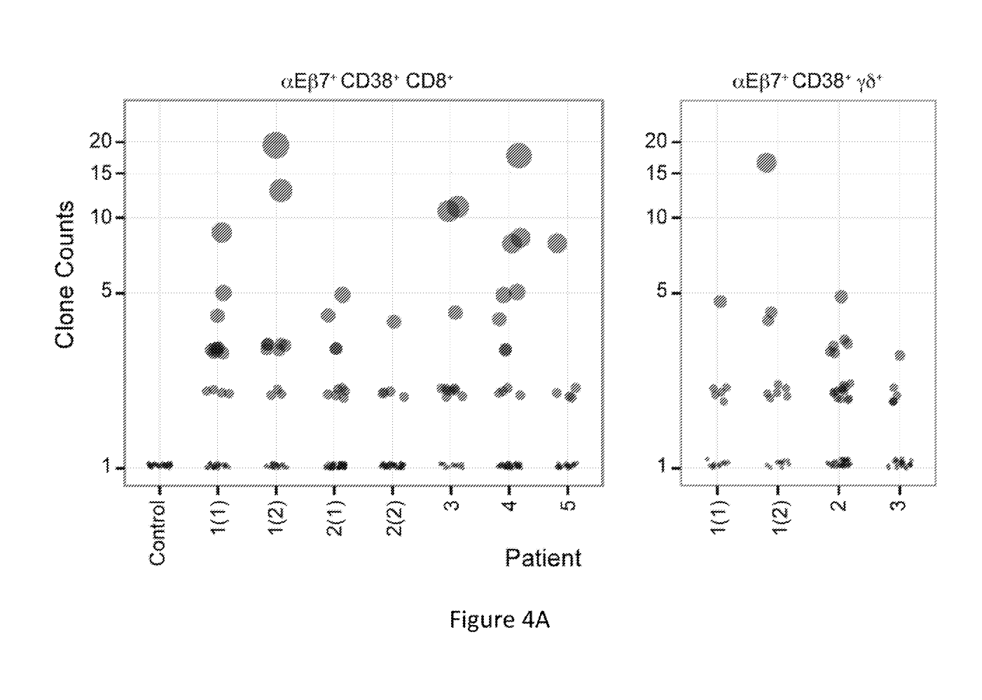

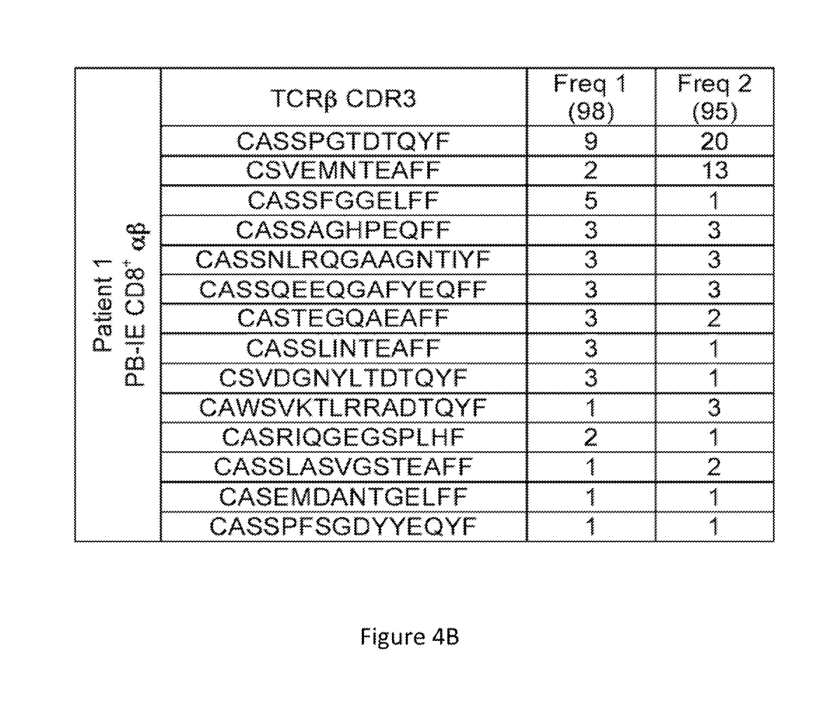

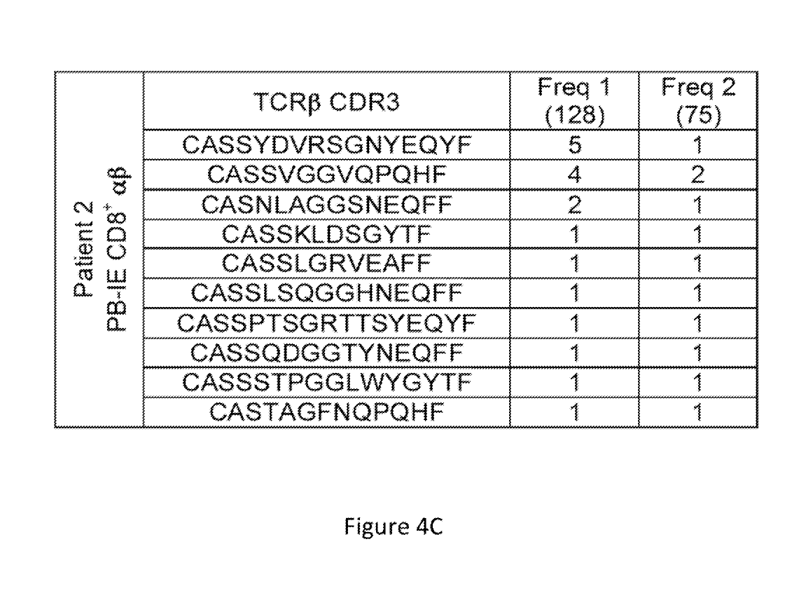

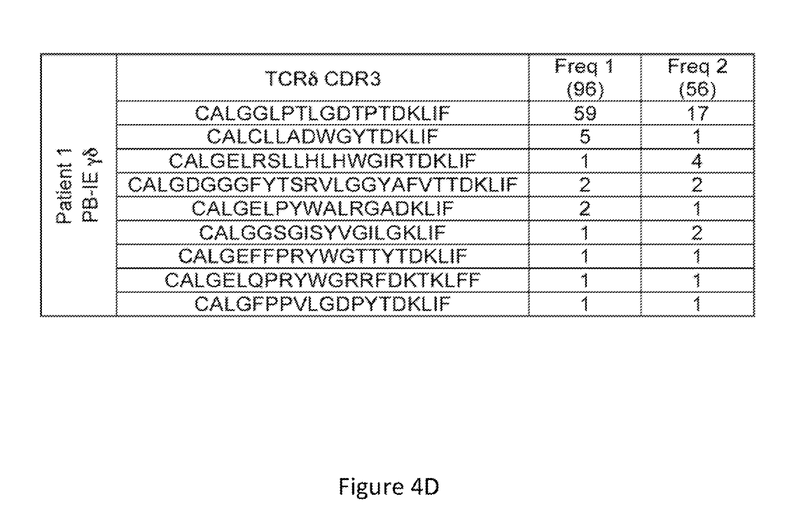

We sequenced TCRβ and TCRδ from single sorted PB-IE CD8 and PB-IE GD cells from celiac patients at day 6 following gluten challenge. We found a high degree of clonality within both CD8 and GD compartments, not observed in peripheral CD8⁺RO⁺ controls (FIG. 4). PB-IE CD8 cells, sequenced in five celiac patients, and PB-IE GD cells, sequenced in three celiac patients, were found to have clonal expansions by TCR sequencing. Celiac patients were rechallenged with gluten after returning to a GFD for at least two months. PB-IE cells were sequenced in these patients to determine whether they would re-elicit cells with a similar TCR repertoire. Indeed, identical TCR sequences and similarity in dominant T cell clones were observed in celiac patients who underwent re-challenge (FIGS. 4B-4D).

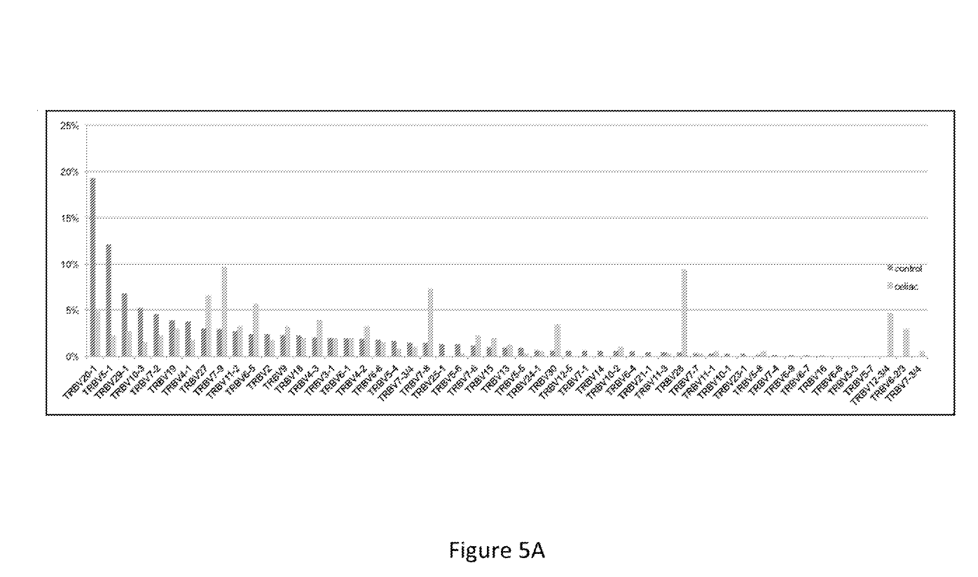

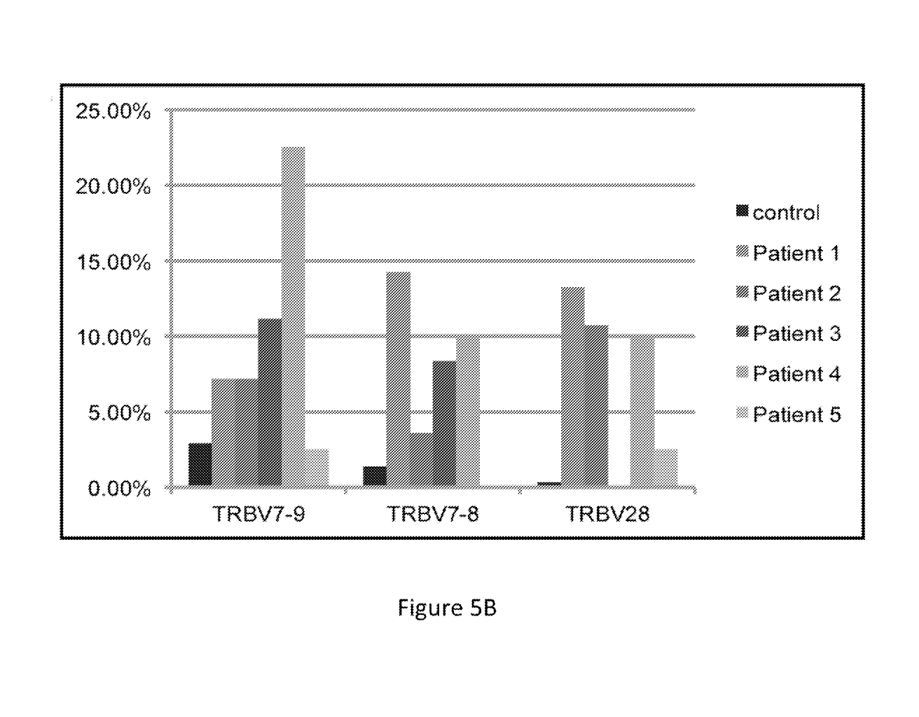

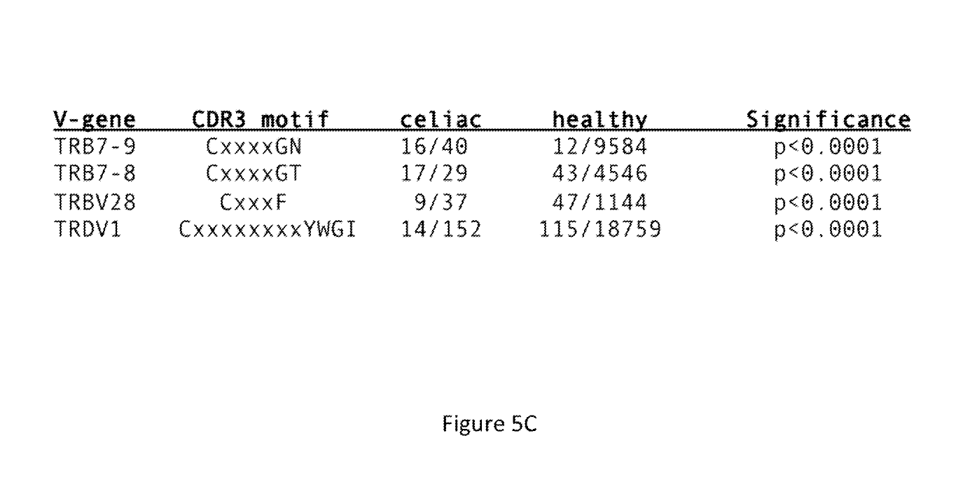

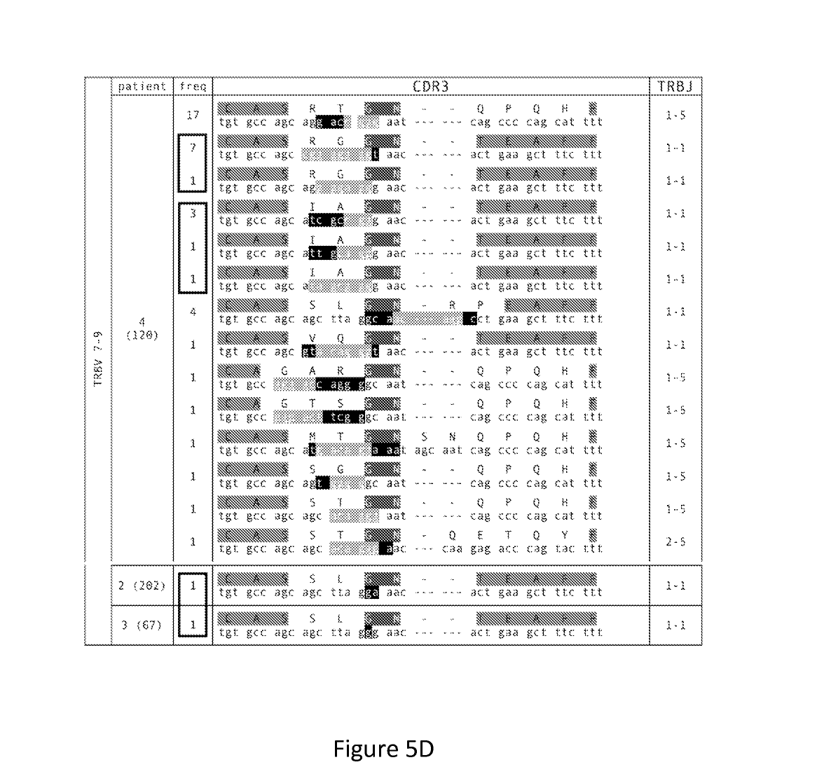

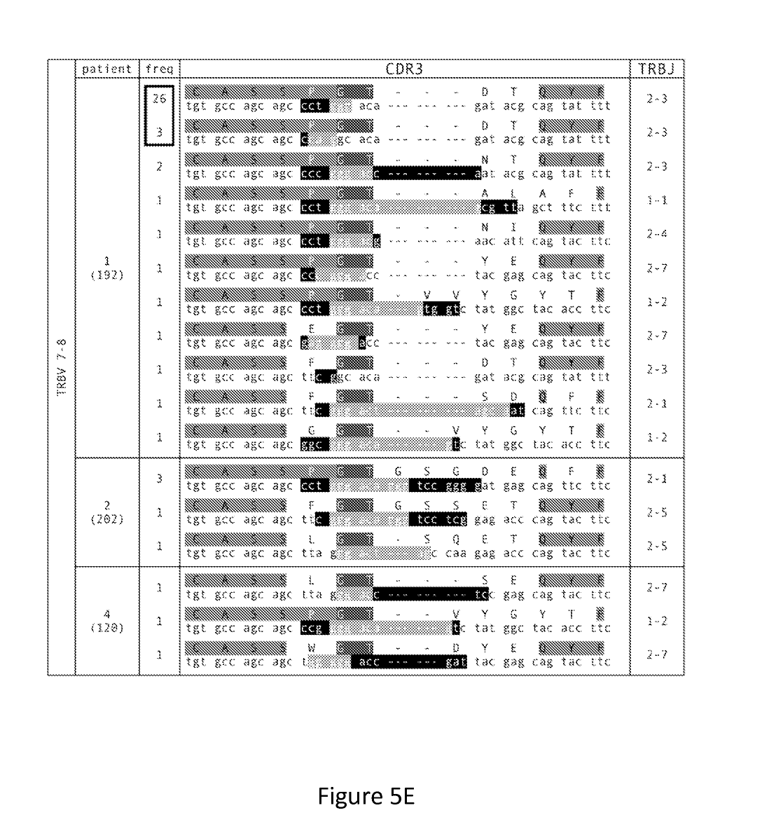

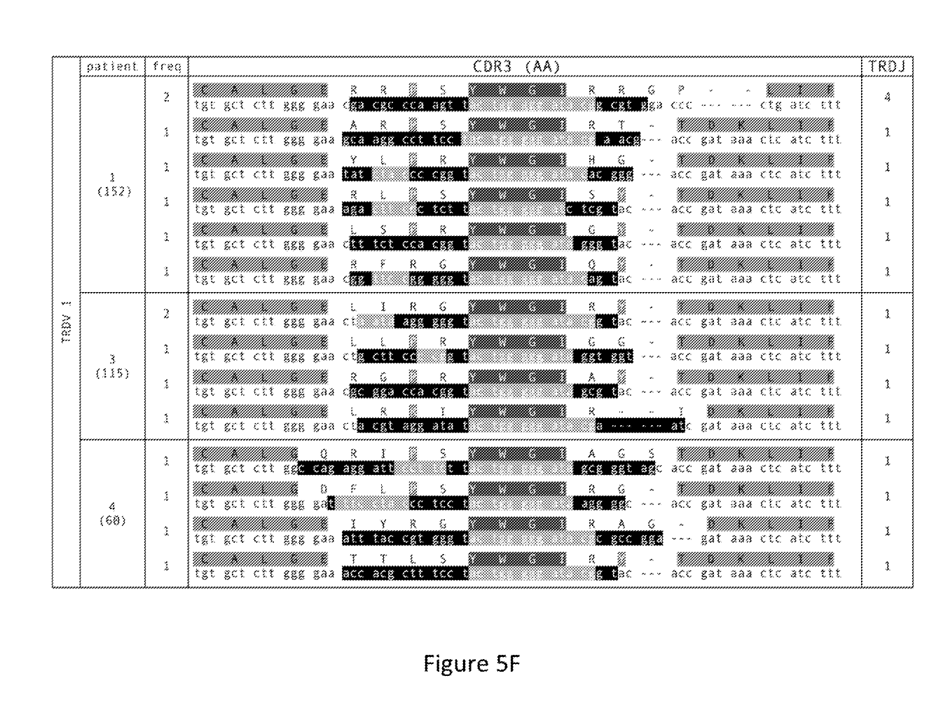

We next evaluated sequences from PB-IE CD8 cells and PB-IE GD cells to determine if we could observe convergence of TCR features within patients or between different patients. Determination of convergence to an unknown antigen within a population of CD8⁺ cells is confounded by the presence of multiple class I alleles within individuals and multiple different TCR motifs that can potentially recognize the same peptide-MHC complex. However, for a particular MHC-peptide, specific CD8⁺ T cell responses are commonly biased toward usage of particular V gene (Kedzierska et al. (2004) Proc. Natl. Acad. Sci. USA 101:4942-4947). It is also appreciated that even individuals of significantly different genetic backgrounds share similar frequency of V gene usage in their TCR repertoire, indicating that skewing within a particular population of cells are not attributable to genetic variation in baseline V gene usage (Ramakrishnan et al. (1992) Scand. J. Immunol. 36:71-78). When assessing the non-redundant TCRβ repertoire of PB-IE CD8 cells in all individuals, we clearly found significant over-representation of particular V regions across multiple celiac PB-IE CD8 samples compared to an unselected healthy control repertoire (FIG. 5A). Most of the peptide specificity of the TCRβ is determined by the CDR3 loop, which is positioned over the antigenic peptide (Kjer-Nielsen et al. (2003) Immunity 18:53-64; Garboczi et al. (1996) Nature 384:134-141). We determined whether convergence could be observed within CDR3β motifs within non-redundant groups of TCR sequences utilizing a common TCRVβ gene. We focused on groups utilizing TCRVβ genes that were clearly overrepresented in a non-redundant sampling within a particular individual, and had members that were clonally expanded. We found four separate examples where identical TCRβ proteins utilized different DNA sequences (FIG. 5D). In three of these instances, the identical convergent TCRβ occurred in the same patient, and at least one clone within these groups was clonally expanded. One identical TCRβ occurred in two different patients. Additionally, within TCRVβ sequences utilizing TRBV7-8 and TRBV7-9, we clearly identified characteristic amino acid motifs (see FIGS. 5C-5F) within the center of CDR3β that were very common within celiac PB-IE cells that were highly uncommon in healthy reference CDR3β sequences (Warren et al. (2011) Genome research 21:790-797). For instance, the GN motif at positions 6-7 within the CDR3 region of TCRβ clones utilizing TRBV7-9 was highly enriched in celiac patients, occurring in 16 out of 40 unique (non-redundant) TCRβ clones, while occurring in only 12/9584 of TCRβ clones utilizing TRBV7-9 within the reference database (p<0.0001) (FIG. 5D). In patient 4, this motif occurred in 14 of 19 unique TCRβ clones, and five of these unique clones utilized distinct VDJ rearrangements to form the same two TCRβ. TCRβ clones utilizing TRBV7-8 similarly converged on a GT motif at position 6-7, which occurred in 17 out of 29 unique (non-redundant) TCRβ clones, while occurring in only 43/4546 TRBV7-8 containing TCRβ clones within the reference database (p<0.0001) (FIG. 5E). The dominant TRBV sequence in patient 1 was formed through two distinct VDJ rearrangements, and both clones were clonally expanded. In all instances where the same TCR was formed using distinct VDJ rearrangements within the same patient, there were at least 2 nucleotide changes within the CDR3 making sequencing or PCR error improbable.

We applied similar analysis to PB-IE GD T cells. Intestinal γδ T cells are appreciated to be heavily biased toward TRDV1 usage (Chowers et al. (1994) J. Exp. Med. 180:183-190). Consistent with this, the majority of PB-IE GD clones from CD patients utilize TRDV1. We analyzed PB-IE CDR3δ sequences utilizing TRDV1 to determine whether convergent motifs could be seen in celiac patients. For comparison, we sequenced TCRδ from bulk small intestinal γδ T cells from a person without celiac disease and bulk blood γδ T cells from nine different patients, obtaining 18579 unique TCRδ sequences utilizing TRDV1. We found that the amino acid motif CxxxxxxxxYWGI (SEQ ID NO:45) was highly enriched within TCRDV1⁺ CDR3δ in PB-IE GD cells compared to reference TCRDV1⁺ γδ T cell sequencing, occurring in all three celiac patients at a total frequency of 14/152 unique sequences while only present in 115/18579 unique reference sequences (FIGS. 5C and 5F).

The high clonality of PB-IE CD8 and PB-IE GD cells, the similarity of TCR repertoire upon repeat challenge, and the conservation of CDR3 motifs in different T cell clones within the same or different patients suggests that both CD8⁺αβ⁺ and γδ⁺ T cells are recruited in an antigen-specific manner in response to dietary gluten.

Discussion

An increase in T-IEL in CD has long been appreciated, and T-IEL are known to be critical in mediating tissue damage and lymphomagenesis. However, the means through which a CD4⁺ T cell response directed against dietary gluten facilitates the activation of IEL has long been a mystery. Models have proposed that IEL might be activated as bystanders downstream of CD4⁺ T cell-mediated inflammation (Jabri & Sollid (2009) Nat. Rev. Immunol. 9:858-870; Meresse et al. (2012) Immunity 36:907-919). Our data suggests that IEL activation and recruitment in CD occurs in parallel with CD4⁺ T cells and is deliberate and antigen-driven.

The function of TCRγδ⁺ IEL (GD-IEL) is poorly understood. An increase in GD-IEL is observed in all stages of CD, and persists even while patients maintain a GFD. In mice, GD-IEL have been shown to have a regulatory role through limiting inflammation and promoting healing of tissue (Chen et al. (2002) Proc. Natl. Acad. Sci. USA 99:14338-14343; Ismail et al. (2009) J. Immunol. 182:3047-3054; Abadie et al. (2012) Semin. Immunopathol. 34:551-566). In human CD, both cytotoxic and anti-inflammatory functions have been attributed to subsets of GD-IEL (Jabri et al. (2000) Gastroenterology 118:867-879; Bhagat et al. (2008) J. Clin. Invest. 118:281-293). Despite the increased presence of GD-IEL in celiac disease even while the patient is on a GFD, our data shows that large amounts of gut-homing TCR γδ⁺ appear to be actively transiting to the gut in response to dietary gluten, possibly in a TCR-dependent manner.

The function of CD8-IEL in CD is much better appreciated, as they are the effectors that directly damage tissue (Jabri & Sollid (2009) Nat. Rev. Immunol. 9:858-870; Meresse et al. (2012) Immunity 36:907-919). It has been suggested that the effector function of CD8 T-IEL in CD might not depend upon antigen. In a manner independent of TCR specificity, CD8 T-IEL have been shown to demonstrate cytotoxicity through stimulation by IL-15 and activation through NK receptors including CD94 and NKG2D (Meresse et al. (2004) Immunity 21:357-366; Meresse et al. (2006) J. Exp. Med. 203:1343-1355). We show that while PB-IE CD8 cells clearly show markers of effector cells and are capable of IFN-γ production, they do not express perforin, CD57 or higher levels of NKG2D. Therefore, it is possible that tissue factors, including IL-15, are further required for cytotoxicity.

However, despite this, our findings show that CD8-IEL express surface markers consistent with effector cells prior to gut recruitment, and suggests that they are initially activated and recruited through an antigen-driven process.

The TCR specificity of IEL in CD has long been enigmatic. Despite extensive study, gluten-derived peptide epitopes recognized by CD8⁺ T cells in CD have not been easily identified and there is no significant genetic association of CD with HLA class I alleles. Therefore, it is generally thought that CD8 T-IEL do not mediate tissue damage through TCR stimulation by gluten. Although gluten recognition by CD8⁺ cells is not a prevailing thought, one group has identified a class I gluten epitope recognized by CD8⁺ T cells isolated from CD mucosa (Mazzarella et al. (2008) Gastroenterology 134:1017-1027). If the PB-IE T cells we describe are responding to gluten, this would imply a rapid and efficient cross presentation of gluten on MHC class I. Besides gluten, other possibilities for CD8 T-IEL ligands include self-antigens or infectious pathogens. The possibility of self-antigen recognition is supported by the observation that CD8-T IEL ultimately lead to tissue damage, and CD is characterized autoantibodies including antibodies to connective tissue (anti-reticulin and endomyseal) and tissue transglutaminase (Jabri & Sollid, supra; Meresse et al. (2012) Immunity 36:907-919). The role of an infectious cofactor in CD has been proposed based on epidemiologic data showing that neonatal infection seems to predispose individuals to the development of CD (Sandberg-Bennich et al. (2002) Acta paediatrica 91:30-33). CD onset has been correlated with evidence of rotavirus infection in children, and in patients treated with IFN-α for hepatitis C (Sandberg-Bennich et al., supra).

It is puzzling how dietary gluten is able to rapidly trigger the activation and gut recruitment of these CD8⁺ TCR αβ⁺ and TCR γδ⁺ T cells that may not recognize gluten themselves. The presence of inflammation has long been postulated to promote the loss of tolerance and prevailing models of CD pathogenesis propose that T-IEL are activated as a result of inflammation that is initiated by gluten-specific CD4⁺ cells. The inflammatory cytokine IL-15 is upregulated within active CD mucosa, and has been implicated in promoting inflammation through diverse means including: impairment of regulatory T cell generation by dendritic cells, promoting NK-like function of CD8 T-IEL, and enabling the expansion of CD8 and GD T-IEL (Meresse et al. (2004) Immunity 21:357-366; DePaolo et al. (2011) Nature 471:220-224). However, we find that appearance of PB-IE cells precisely parallels the appearance of gluten-reactive CD4⁺ cells in blood, rather than occurring later. Also, although increased numbers of IEL and mildly increased levels of IL-15 are present in celiac patients on a GFD (Di Sabatino et al. (2006) Gut 55:469-477), the recruitment we describe precedes significant intestinal inflammation and tissue damage, which only reliably occurs histologically after 2-4 weeks of continuous gluten exposure (Leffler et al. (2013) Gut 62(7):996-1004).

This process through which these three T cell subsets are synchronously mobilized and recruited to the tissue clearly has implications in immunity to infectious pathogens, and the development of autoimmunity in CD likely represents a distortion of processes that are meant to be protective. Due to the well-established dependence of CD on the CD4⁺ T cell response, the celiac T cell cascade that we describe presumably depends upon gluten-specific CD4⁺ T cells. Multiple aspects of the effector CD8⁺ T cell responses to viruses have been shown to depend upon CD4⁺ T cell help; including primary effector responses, the generation of memory, and recruitment to sites of infection (Nakanishi et al. (2009) Nature 462:510-513; Janssen et al. (2003) Nature 421:852-856; Shedlock & Shen (2003) Science 300:337-339; Sun & Bevan (2003) Science 300:339-342). This process has been termed licensing, referring to ability of CD4⁺ T cells to “license” cognate effector CD8⁺ T cell responses. Here, we describe a process whereby CD4⁺ T cells may be “licensing” CD8⁺ T cells to cause human autoimmunity. This process may share mechanisms with the process of licensing that have been described to coordinate CD4⁺ and effector T cell responses to viruses.

Like CD, most autoimmune diseases with HLA associations are associated with MHC class II alleles, including Type 1 diabetes, multiple sclerosis, rheumatoid arthritis, and ulcerative colitis (Trowsdale (2011) Immunology letters 137:1-8). Despite the association of these diseases with class II alleles rather than class I alleles, CD8⁺ effector cells play an important role in the pathogenesis of these diseases and are clearly present at the site of inflammation. We speculate that the T cell cascade we observe in which a CD4⁺ T cell response to an initiating antigen potentially enables a parallel activation of effector CD8⁺ and TCR γδ⁺ T cells is relevant in other autoimmune diseases.

Analysis of gut homing, activated cells within the peripheral blood may have clinical utility in the diagnosis of CD. An estimated 1.6 million Americans follow a GFD without an established diagnosis of CD (Rubio-Tapia et al. (2012) Am. J. Gastroenterol. 107:1538-1544). Currently available tests, including antibody tests and intestinal biopsy, are inaccurate in patients on a GFD and require prolonged (2-4 weeks) gluten exposure, which is often intolerable to patients, precluding an accurate diagnosis (Leffler et al., supra). Our study shows promise in the reliable clinical diagnosis of CD with only short-term gluten exposure.

TABLE 1
Quantification of peripheral blood αEβ7+ CD38+ CD8+ and
αEβ7+ CD38+ γδ T cells in celiac patients and control individuals
following gluten challenge. All six celiac patients but none of the
controls exhibit clear induction of peripheral blood αEβ7⁺
CD38⁺ CD8⁺ and αEβ7⁺ CD38⁺ γδ T cells on day 6 following
oral gluten challenge. Numbers indicate αEβ7⁺
CD38⁺ CD8⁺ or αEβ7⁺ CD38⁺ γδ T cells as a
percentage of total blood CD8⁺ or γδ T cells.

| | Day 0 | Day 6 | Day 0 | Day 6 |
|---|---|---|---|---|
| | (% αEβ7+, | (% αEβ7+, | (% αEβ7+, | (% αEβ7+, |
| | CD38+, | CD38+, | CD38+, | CD38+, |
| | CD8+/ | CD8+/ | γδ+/ | γδ+/ |
| | total CD8+) | total CD8+) | total γδ+) | total γδ+) |
| Control 1 | 0.04% | 0.01% | 0.01% | 0.00% |
| Control 2 | 0.00% | 0.03% | 0.00% | 0.00% |
| Control 3 | 0.03% | 0.01% | 0.00% | 0.00% |
| Control 4 | 0.01% | 0.02% | 0.00% | 0.00% |
| Control 5 | 0.01% | 0.01% | 0.00% | 0.00% |
| Median | 0.01% | 0.01% | 0.00% | 0.00% |
| (Controls) | | | | |
| Celiac 1 | 0.01% | 1.02% | 0.00% | 12.88% |
| Celiac 2 | 0.08% | 1.69% | 0.04% | 0.93% |
| Celiac 3 | 0.59% | 10.17% | 5.58% | 18.61% |
| Celiac 4 | 0.03% | 0.84% | 0.11% | 1.81% |
| Celiac 5 | 0.08% | 1.69% | 0.04% | 0.93% |
| Celiac 6 | 0.00% | 0.37% | 0.01% | 0.06% |
| Celiac 1 | 0.01% | 1.20% | 0.00% | 6.81% |
| (rechallenge) | | | | |
| Celiac 2 | 0.01% | 1.24% | 0.05% | 1.24% |
| (rechallenge) | | | | |
| Median (Celiac) | 0.02% | 1.11% | 0.03% | 1.52% |

TABLE 2
Single-cell TCR sequencing of alpha-II-gliadin
tetramer positive T cells shows most clones
utilize TRBV7-2 and share a consensus arginine
at position 5. CDR3β sequences from 2 patients
with indicated V and J usage and frequency.

| | v gene | J gene | CDR3 | Freq |
|---|---|---|---|---|
| Patient 1 | TRBV7-2 | TRBJ2-3 | ASSIRSTDTQYF | 8 |
| | | | (SEQ ID NO: 7) | |
| | TRBV5-1 | TRBJ2-3 | ASSLGQPSTDTQYF | 4 |
| | | | (SEQ ID NO: 8) | |
| | TRBV7-2 | TRBJ2-7 | ASSIRTSGAHEQYF | 3 |
| | | | (SEQ ID NO: 9) | |
| | TRBV7-2 | TRBJ2-7 | ASSLRAGGSHEQYF | 3 |
| | | | (SEQ ID NO: 10) | |
| | TRBV6-3 | TRBJ2-3 | ASSFQRGAADTQYF | 2 |
| | | | (SEQ ID NO: 11) | |
| | TRBV7-2 | TRBJ2-3 | ASSFRVGAVDTQYF | 2 |
| | | | (SEQ ID NO: 12) | |
| | TRBV7-2 | TRBJ2-3 | ASSIRWTDTQYF | 2 |
| | | | (SEQ ID NO: 13) | |
| | TRBV4-3 | TRBJ2-3 | ASSQVYGGDTQYF | 2 |
| | | | (SEQ ID NO: 14) | |
| | TRBV2 | TRBJ2-3 | ASSWTGGHTDTQYF | 2 |
| | | | (SEQ ID NO: 15) | |
| | TRBV10-2 | TRBJ2-7 | ASSEQREGEQYF | 1 |
| | | | (SEQ ID NO: 16) | |
| | TRBV7-2 | TRBJ2-3 | ASSIRAGGTDTQYF | 1 |
| | | | (SEQ ID NO: 17) | |
| | TRBV7-2 | TRBJ2-3 | ASSIRDTDTQYF | 1 |
| | | | (SEQ ID NO: 18) | |
| | TRBV7-2 | TRBJ2-7 | ASSIRTGDHEQYF | 1 |
| | | | (SEQ ID NO: 19) | |
| | TRBV7-2 | TRBJ2-5 | ASSIRTGGETQYF | 1 |
| | | | (SEQ ID NO: 20) | |
| | TRBV7-2 | TRBJ2-3 | ASSIRTTDTQYF | 1 |
| | | | (SEQ ID NO: 21) | |
| | TRBV7-2 | TRBJ2-3 | ASSIRYTDTQYF | 1 |
| | | | (SEQ ID NO: 22) | |
| | TRBV7-2 | TRBJ2-3 | ASSLASYGDTQYF | 1 |
| | | | (SEQ ID NO: 23) | |
| | TRBV7-2 | TRBJ2-3 | ASSLRFTDTQYF | 1 |
| | | | (SEQ ID NO: 24) | |
| | TRBV7-2 | TRBJ2-5 | ASSLVAYSGETQYF | 1 |
| | | | (SEQ ID NO: 25) | |
| | TRBV7-2 | TRBJ2-3 | ASSTRTTDTQYF | 1 |
| | | | (SEQ ID NO: 26) | |
| | TRBV7-2 | TRBJ2-3 | ASSTRWSDTQYF | 1 |
| | | | (SEQ ID NO: 27) | |
| | TRBV7-3 | TRBJ2-3 | ASSVRFTDTQYF | 1 |
| | | | (SEQ ID NO: 28) | |
| | TRBV7-2 | TRBJ2-3 | ATSIRFTDTQYF | 1 |
| | | | (SEQ ID NO: 29) | |
| | | | total | 42 |
| Patient 2 | TRBV7-2 | TRBJ2-3 | ASSVRFTDTQYF | 6 |
| | | | (SEQ ID NO: 30) | |
| | TRBV7-2 | TRBJ2-7 | ASSIRQGGNHEQYF | 5 |
| | | | (SEQ ID NO: 31) | |
| | TRBV7-2 | TRBJ2-3 | ASSFRSTDTQYF | 3 |
| | | | (SEQ ID NO: 32) | |
| | TRBV4-1 | TRBJ2-5 | ASSQDGQGPETQYF | 3 |
| | | | (SEQ ID NO: 33) | |
| | TRBV7-2 | TRBJ2-3 | ASSVRSTDTQYF | 3 |
| | | | (SEQ ID NO: 34) | |
| | TRBV7-2 | TRBJ2-3 | ASSFRVSDTQYF | 2 |
| | | | (SEQ ID NO: 35) | |
| | TRBV7-2 | TRBJ2-3 | ASSIRNTDTQYF | 2 |
| | | | (SEQ ID NO: 36) | |
| | TRBV7-2 | TRBJ2-3 | ASSLRAGGVDTQYF | 2 |
| | | | (SEQ ID NO: 37) | |
| | TRBV4-2 | TRBJ2-1 | ASSQPHRGDEQFF | 2 |
| | | | (SEQ ID NO: 38) | |
| | TRBV7-2 | TRBJ2-3 | ASSARFTDTQYF | 1 |
| | | | (SEQ ID NO: 39) | |
| | TRBV7-2 | TRBJ2-3 | ASSFRTSDTQYF | 1 |
| | | | (SEQ ID NO: 40) | |
| | TRBV7-2 | TRBJ2-3 | ASSIRSTDTQYF | 1 |
| | | | (SEQ ID NO: 41) | |
| | TRBV7-2 | TRBJ2-3 | ASSLRSGDTQYF | 1 |
| | | | (SEQ ID NO: 42) | |
| | TRBV7-2 | TRBJ2-5 | ASSLVAWETQYF | 1 |
| | | | (SEQ ID NO: 43) | |
| | TRBV7-2 | TRBJ2-3 | ASSVRGGEADTQYF | 1 |
| | | | (SEQ ID NO: 44) | |
| | | | total | 34 |

TABLE 3
Summary of single-cell TCRβ and TCRδ sequencing:
CDR3β and CDR3δ sequences from all patients
tested with indicated V and J usage and
frequency.

| | | | |
|---|---|---|---|
| CDR3 | Freq | TRBV | TRBJ |

Table 3a
Patient 1, challenge 1
PB-IE CD8 TCRβ (98)

| | | | |
|---|---|---|---|
| CASSPGTDTQYF | 9 | TRBV7- | TRBJ2-3 |
| (SEQ ID NO: 46) | | 8 | |
| CASSLPPRGGGYEQYF | 1 | TRBV28 | TRBJ2-7 |
| (SEQ ID NO: 47) | | | |
| CASSFGGELFF | 5 | TRBV7- | TRBJ2-2 |
| (SEQ ID NO: 48) | | 2 | |
| CASSPEDPYTDTQYF | 1 | TRBV13 | TRBJ2-3 |
| (SEQ ID NO: 49) | | | |
| CASAGNYEKLFF | 4 | TRBV28 | TRBJ1-4 |
| (SEQ ID NO: 50) | | | |
| CASSPEREVYNEQFF | 1 | TRBV6- | TRBJ2-1 |
| (SEQ ID NO: 51) | | 2/3 | |
| CASSAGHPEQFF | 3 | TRBV7- | TRBJ2-1 |
| (SEQ ID NO: 52) | | 8 | |
| CASSPFSGDYYEQYF | 1 | TRBV18 | TRBJ2-7 |
| (SEQ ID NO: 53) | | | |
| CASSLINTEAFF | 3 | TRBV11- | TRBJ1-1 |
| (SEQ ID NO: 54) | | 2 | |
| CASSPGTNIQYF | 1 | TRBV7- | TRBJ2-4 |
| (SEQ ID NO: 55) | | 8 | |
| CASSNLRQGAAGNTIYF | 3 | TRBV28 | TRBJ1-3 |
| (SEQ ID NO: 56) | | | |
| CASSPGTVVYGYTF | 1 | TRBV7- | TRBJ1-2 |
| (SEQ ID NO: 57) | | 8 | |
| CASSQEEQGAFYEQFF | 3 | TRBV4- | TRBJ2-1 |
| (SEQ ID NO: 58) | | 3 | |
| CASSPGTYEQYF | 1 | TRBV7- | TRBJ2-7 |
| (SEQ ID NO: 59) | | 8 | |
| CASTEGQAEAFF | 3 | TRBV7- | TRBJ1-1 |
| (SEQ ID NO: 60) | | 8 | |
| CASSPGVYEQYF | 1 | TRBV7- | TRBJ2-7 |
| (SEQ ID NO: 61) | | 8 | |
| CATSGTSGYNEQFF | 3 | TRBV7- | TRBJ2-1 |
| (SEQ ID NO: 62) | | 9 | |
| CASSPSNTGELFF | 1 | TRBV6- | TRBJ2-2 |
| (SEQ ID NO: 63) | | 2/3 | |
| CSVDGNYLTDTQYF | 3 | TRBV29- | TRBJ2-3 |
| (SEQ ID NO: 64) | | 1 | |
| CASSQAGALWDYGYTF | 1 | TRBV4- | TRBJ1-2 |
| (SEQ ID NO: 65) | | 1 | |
| CASRIQGEGSPLHF | 2 | TRBV7- | TRBJ1-6 |
| (SEQ ID NO: 66) | | 9 | |
| CASSQGIRSEYEQYF | 1 | TRBV3- | TRBJ2-7 |
| (SEQ ID NO: 67) | | 1 | |
| CASSPGTNTQYF | 2 | TRBV7- | TRBJ2-3 |
| (SEQ ID NO: 68) | | 8 | |
| CASSQLPVNSPLHF | 1 | TRBV3- | TRBJ1-6 |
| (SEQ ID NO: 69) | | 1 | |
| CASSQDLGDYGYTF | 2 | TRBV4- | TRBJ1-2 |
| (SEQ ID NO: 70) | | 3 | |
| CASSSGLATDTQYF | 1 | TRBV6- | TRBJ2-3 |
| (SEQ ID NO: 71) | | 5 | |
| CSVEMNTEAFF | 2 | TRBV9- | TRBJ1-1 |
| (SEQ ID NO: 72) | | 1 | |
| CASSTGHMEDTQYF | 1 | TRBV19 | TRBJ2-3 |
| (SEQ ID NO: 73) | | | |
| CAISDPPLATEAFF | 1 | TRBV10- | TRBJ1-1 |
| (SEQ ID NO: 74) | | 3 | |
| CASSTGVSGANVLTF | 1 | TRBV18 | TRBJ2-6 |
| (SEQ ID NO: 75) | | | |
| CAISSGQVPEQFF | 1 | TRBV10- | TRBJ2-1 |
| (SEQ ID NO: 76) | | 3 | |
| CASSVEGGMGEKLFF | 1 | TRBV9 | TRBJ1-4 |
| (SEQ ID NO: 77) | | | |
| CASEMDANTGELFF | 1 | TRBV28 | TRBJ2-2 |
| (SEQ ID NO: 78) | | | |
| CASSVGAGVNSYEQYF | 1 | TRBV9 | TRBJ2-7 |
| (SEQ ID NO: 79) | | | |
| CASKLGGATEAFF | 1 | TRBV6- | TRBJ1-1 |
| (SEQ ID NO: 80) | | 1 | |
| CASSVRAGTGTYEQYF | 1 | TRBV9 | TRBJ2-7 |
| (SEQ ID NO: 81) | | | |
| CASNQGQGVETQYF | 1 | TRBV12- | TRBJ2-5 |
| (SEQ ID NO: 82) | | 3/4 | |
| CASSVSTGSYEQYF | 1 | TRBV7- | TRBJ2-7 |
| (SEQ ID NO: 83) | | 8 | |
| CASSEGTYEQYF | 1 | TRBV7- | TRBJ2-7 |
| (SEQ ID NO: 84) | | 8 | |
| CASSWDRATNEKLFF | 1 | TRBV7- | TRBJ1-4 |
| (SEQ ID NO: 85) | | 9 | |
| CASSFGTDTQYF | 1 | TRBV7- | TRBJ2-3 |
| (SEQ ID NO: 86) | | 8 | |
| CASSYAPTGNYGYTF | 1 | TRBV6- | TRBJ1-2 |
| (SEQ ID NO: 87) | | 5 | |
| CASSFGTSDQFF | 1 | TRBV7- | TRBJ2-1 |
| (SEQ ID NO: 88) | | 8 | |
| CASSYKRGPGELFF | 1 | TRBV6- | TRBJ2-2 |
| (SEQ ID NO: 89) | | 5 | |
| CASSFLGTYEQYF | 1 | TRBV28 | TRBJ2-7 |
| (SEQ ID NO: 90) | | | |
| CASSYSMASGGAQETQYF | 1 | TRBV6- | TRBJ2-5 |
| (SEQ ID NO: 91) | | 5 | |
| CASSFPNPTFEAFF | 1 | TRBV28 | TRBJ1-1 |
| (SEQ ID NO: 92) | | | |
| CASSYTAGSNQPQHF | 1 | TRBV6- | TRJ1-5 |
| (SEQ ID NO: 93) | | 6 | |
| CASSFRGQGNEQYF | 1 | TRBV28 | TRBJ2-7 |
| (SEQ ID NO: 94) | | | |
| CASSYWEEGGGAFF | 1 | TRBV6- | TRBJ1-1 |
| (SEQ ID NO: 95) | | 2/3 | |
| CASSFTGSSYEQYF | 1 | TRBV28 | TRBJ2-7 |
| (SEQ ID NO: 96) | | | |
| CASSYWGPMNTEAFF | 1 | TRBV6- | TRBJ1-1 |
| (SEQ ID NO: 97) | | 2/3 | |
| CASSGGTVYGYTF | 1 | TRBV7- | TRJ1-2 |
| (SEQ ID NO: 98) | | 8 | |
| CAWSVKTLRRADTQYF | 1 | TRBV30 | TRBJ2-3 |
| (SEQ ID NO: 99) | | | |
| CASSGSGGVTGELFF | 1 | TRBV6- | TRBJ2-2 |
| (SEQ ID NO: 100) | | 5 | |
| CSAAGHFYEQYF | 1 | TRBV20- | TRBJ2-7 |
| (SEQ ID NO: 101) | | 1 | |
| CASSLASVGSTEAFF | 1 | TRBV7- | TRBJ1-1 |
| (SEQ ID NO: 102) | | 2 | |
| CSAMTQEDYAFF | 1 | TRBV20- | TRBJ2-1 |
| (SEQ ID NO: 103) | | 1 | |
| CASSLGTGGYNEQFF | 1 | TRBV7- | TRBJ2-1 |
| (SEQ ID NO: 104) | | 2 | |
| CSARDFSLRTGELFF | 1 | TRBV20- | TRBJ2-2 |
| (SEQ ID NO: 105) | | 1 | |
| CASSLLGLTGELFF | 1 | TRBV27 | TRBJ2-2 |
| (SEQ ID NO: 106) | | | |
| CSARDMFGGHVSGNTIYF | 1 | TRBV20- | TRJ1-3 |
| (SEQ ID NO: 107) | | 1 | |
| CASSLNLGQNTEAFF | 1 | TRBV7- | TRBJ1-1 |
| (SEQ ID NO: 108) | | 3/4 | |
| CSVDGLAGITDTQYF | 1 | TRBV29- | TRBJ2-3 |
| (SEQ ID NO: 109) | | 1 | |
| CASSLPLGQGNQPQHF | 1 | TRBV28 | TRJ1-5 |
| (SEQ ID NO: 110) | | | |

Patient 1, Challenge 2
PB-IE CD8 TCRβ (94)

| | | | |
|---|---|---|---|
| CASSPGTDTQYF | 20 | TRBV7- | TRBJ2-3 |
| (SEQ ID NO: 46) | | 8 | |
| CASSLTRQGGEGSPLHF | 1 | TRBV28 | TRJ1-6 |
| (SEQ ID NO: 111) | | | |
| CSVEMNTEAFF | 13 | TRBV29- | TRBJ1-1 |
| (SEQ ID NO: 72) | | 1 | |
| CASSLVADSYNEQFF | 1 | TRBV7- | TRBJ2-1 |
| (SEQ ID NO: 112) | | 2 | |
| CASSLLAGGAEQFF | 3 | TRBV4- | TRBJ2-1 |
| (SEQ ID NO: 113) | | 3 | |
| CASSLVGPGDTQYF | 1 | TRBV28 | TRBJ2-3 |
| (SEQ ID NO: 114) | | | |
| CASSNLRQGAAGNTIYF | 3 | TRBV28 | TRJ1-3 |
| (SEQ ID NO: 56) | | | |
| CASSPFSGDYYEQYF | 1 | TRBV18 | TRBJ2-7 |
| (SEQ ID NO: 53) | | | |
| CASSQEEQGAFYEQFF | 3 | TRBV4- | TRBJ2-1 |
| (SEQ ID NO: 58) | | 3 | |
| CASSPGTALAFF | 1 | TRBV7- | TRBJ1-1 |
| (SEQ ID NO: 115) | | 8 | |
| CASSSSDRATDTQYF | 3 | TRBV7- | TRBJ2-3 |
| (SEQ ID NO: 116) | | 9 | |
| CASSQDGGARQHF | 1 | TRBV4- | TRJ1-5 |
| (SEQ ID NO: 117) | | 3 | |
| CAWSVKTLRRADTQYF | 3 | TRBV30 | TRBJ2-3 |
| (SEQ ID NO: 99) | | | |
| CASSQDSGGARNNEQFF | 1 | TRBV3- | TRBJ2-1 |
| (SEQ ID NO: 118) | | 1 | |
| CASSAGHPEQFF | 3 | TRBV7- | TRBJ2-1 |
| (SEQ ID NO: 52) | | 8 | |
| CASSQDWALGWGYGYTF | 1 | TRBV4- | TRJ1-2 |
| (SEQ ID NO: 119) | | 2 | |
| CASSLASVGSTEAFF | 2 | TRBV7- | TRBJ1-1 |
| (SEQ ID NO: 102) | | 2 | |
| CASSQLTQNTEAFF | 1 | TRBV4- | TRBJ1-1 |
| (SEQ ID NO: 120) | | 3 | |
| CASSYRQAGYEQYF | 2 | TRBV11- | TRBJ2-7 |
| (SEQ ID NO: 121) | | 1 | |
| CASSQPFVGSGNTIYF | 1 | TRBV4- | TRJ1-3 |
| (SEQ ID NO: 122) | | 1 | |
| CASTEGQAEAFF | 2 | TRBV7- | TRBJ1-1 |
| (SEQ ID NO: 60) | | 8 | |
| CASSRDWGETQYF | 1 | TRBV4- | TRBJ2-5 |
| (SEQ ID NO: 123) | | 3 | |
| CASSLPTAVTEAFF | 1 | TRBV7- | TRBJ1-1 |
| (SEQ ID NO: 124) | | 9 | |
| CASSRGLAGESTDTQYF | 1 | TRBV6- | TRBJ2-3 |
| (SEQ ID NO: 125) | | 5 | |
| CASEMDANTGELFF | 1 | TRBV28 | TRBJ2-2 |
| (SEQ ID NO: 78) | | | |
| CASSRPASYEQYF | 1 | TRBV7- | TRBJ2-7 |
| (SEQ ID NO: 126) | | 9 | |
| CASRIQGEGSPLHF | 1 | TRBV7- | TRBJ1-6 |
| (SEQ ID NO: 66) | | 9 | |
| CASSSGADLYGYTF | 1 | TRBV11- | TRJ1-2 |
| (SEQ ID NO: 127) | | 2 | |
| CASSDRGFPSYEQYF | 1 | TRBV6- | TRBJ2-7 |
| (SEQ ID NO: 128) | | 3 | |
| CASSSGLSYNEQFF | 1 | TRBV27 | TRBJ2-1 |
| (SEQ ID NO: 129) | | | |
| CASSFGGELFF | 1 | TRBV7- | TRBJ2-2 |
| (SEQ ID NO: 48) | | 2 | |
| CASSSGTLETQYF | 1 | TRBV6- | TRBJ2-5 |
| (SEQ ID NO: 130) | | 5 | |
| CASSFGNQPQHF | 1 | TRBV7- | TRJ1-5 |
| (SEQ ID NO: 131) | | 8 | |
| CASSSSSGSTYEQYF | 1 | TRBV7- | TRBJ2-7 |
| (SEQ ID NO: 132) | | 2 | |
| CASSFSLAGWSYNEQFF | 1 | TRBV28 | TRBJ2-1 |
| (SEQ ID NO: 133) | | | |
| CASSSWTGTNTEAFF | 1 | TRBV7- | TRBJ1-1 |
| (SEQ ID NO: 134) | | 9 | |
| CASSFSNGENTDTQYF | 1 | TRBV12- | TRBJ2-3 |
| (SEQ ID NO: 135) | | 3 | |
| CASSWLAGGPAGELFF | 1 | TRBV28 | TRBJ2-2 |
| (SEQ ID NO: 136) | | | |
| CASSHLGGGNTIYF | 1 | TRBV4- | TRJ1-3 |
| (SEQ ID NO: 137) | | 1 | |
| CASSYATGTPSSYNEQFF | 1 | TRBV6- | TRBJ2-1 |
| (SEQ ID NO: 138) | | 2 | |
| CASSLAGDSYNEQFF | 1 | TRBV7- | TRBJ2-1 |
| (SEQ ID NO: 139) | | 2 | |
| CASSYGSDSYNEQFF | 1 | TRBV6- | TRBJ2-1 |
| (SEQ ID NO: 140) | | 5 | |
| CASSLGQGTPDTQYF | 1 | TRBV7- | TRBJ2-3 |
| (SEQ ID NO: 141) | | 6 | |
| CSARVSGSLYEQYF | 1 | TRBV20- | TRBJ2-7 |
| (SEQ ID NO: 142) | | 1 | |
| CASSLINTEAFF | 1 | TRBV11- | TRBJ1-1 |
| (SEQ ID NO: 54) | | 2 | |
| CSVDGNYLTDTQYF | 1 | TRBV29- | TRBJ2-3 |
| (SEQ ID NO: 64) | | 1 | |
| CASSLSLAGDTGELFF | 1 | TRBV11- | TRBJ2-2 |
| (SEQ ID NO: 143) | | 2 | |
| CSVEMSGGDYEQYF | 1 | TRBV29- | TRBJ2-7 |
| (SEQ ID NO: 144) | | 1 | |

Table 3b
Patient 2, Challenge 1
PB-IE CD8 TCRβ (127)

| | | | |
|---|---|---|---|
| CASSYDVRSGNYEQYF | 5 | TRBV6- | TRBJ2-7 |
| (SEQ ID NO: 145) | | 6 | |
| CASSLSADRDGGYTF | 1 | TRBV30 | TRBJ2-1 |
| (SEQ ID NO: 146) | | | |
| CASSPSDPSDTQYF | 4 | TRBV11- | TRBJ2-3 |
| (SEQ ID NO: 147) | | 2 | |
| CASSLSQGGHNEQFF | 1 | TRBV7- | TRBJ2-1 |
| (SEQ ID NO: 148) | | 6 | |
| CASSPGTGSGDEQFF | 3 | TRBV7- | TRBJ2-1 |
| (SEQ ID NO: 149) | | 8 | |
| CASSLVPPGGFSYEQYF | 1 | TRBV5- | TRBJ2-7 |
| (SEQ ID NO: 150) | | 1 | |
| CASSVGGVQPQHF | 3 | TRBV12- | TRBJ1-5 |
| (SEQ ID NO: 151) | | 4 | |
| CASSMGQGNSGETQYF | 1 | TRBV9 | TRBJ2-5 |
| (SEQ ID NO: 152) | | | |
| CASNLAGGSNEQFF | 2 | TRBV6- | TRBJ2-1 |
| (SEQ ID NO: 153) | | 5 | |
| CASSPGGAGYTF | 1 | TRBV9 | TRBJ1-2 |
| (SEQ ID NO: 154) | | | |
| CASSFGQVTYEQYF | 2 | TRBV28 | TRBJ2-7 |
| (SEQ ID NO: 155) | | | |
| CASSPGGWSYEQYF | 1 | TRBV27 | TRBJ2-7 |
| (SEQ ID NO: 156) | | | |
| CASSLGGAETQYF | 2 | TRBV7- | TRBJ2-5 |
| (SEQ ID NO: 157) | | 9 | |
| CASSPGQGNNSPLHF | 1 | TRBV7- | TRBJ1-6 |
| (SEQ ID NO: 158) | | 6 | |
| CASSQDWGDYGYTF | 2 | TRBV4- | TRBJ1-2 |
| (SEQ ID NO: 159) | | 2 | |
| CASSPISRDRNTGELFF | 1 | TRBV18 | TRBJ2-2 |
| (SEQ ID NO: 160) | | | |
| CASSSEQDRGSENTIYF | 2 | TRBV7- | TRBJ1-3 |
| (SEQ ID NO: 161) | | 3 | |
| CASSPLGSGTEAFF | 1 | TRBV28 | TRBJ1-1 |
| (SEQ ID NO: 162) | | | |
| CASSWSGYEQYF | 2 | TRBV7- | TRBJ2-7 |
| (SEQ ID NO: 163) | | 9 | |
| CASSPNPNTGELFF | 1 | TRBV7- | TRBJ2-2 |
| (SEQ ID NO: 164) | | 8 | |
| CASSWTGNEQFF | 1 | TRBV6- | TRBJ2-1 |
| (SEQ ID NO: 165) | | 6 | |
| CASSPPDRGYDNEQFF | 1 | TRBV6- | TRBJ2-1 |
| (SEQ ID NO: 166) | | 5 | |
| CAIRATGLAGVDTGELFF | 1 | TRBV10- | TRBJ2-2 |
| (SEQ ID NO: 167) | | 3 | |
| CASSPPLTEAFF | 1 | TRBV18 | TRBJ1-1 |
| (SEQ ID NO: 168) | | | |
| CAISEQEYGTEAFF | 1 | TRBV10- | TRBJ1-1 |
| (SEQ ID NO: 169) | | 3 | |
| CASSPRLAGAKDTQYF | 1 | TRBV19 | TRBJ2-3 |
| (SEQ ID NO: 170) | | | |
| CAISFGTGEAPRGYTF | 1 | TRBV10- | TRBJ1-2 |
| (SEQ ID NO: 171) | | 3 | |
| CASSPRSAGGPYEQYF | 1 | TRBV27 | TRBJ2-7 |
| (SEQ ID NO: 172) | | | |
| CAISPDRVTFEVFF | 1 | TRBV10- | TRBJ1-1 |
| (SEQ ID NO: 173) | | 3 | |
| CASSPTSGRTTSYEQYF | 1 | TRBV4- | TRBJ2-7 |
| (SEQ ID NO: 174) | | 1 | |
| CALWGGAYEQYF | 1 | TRBV30 | TRBJ2-7 |
| (SEQ ID NO: 175) | | | |
| CASSQDGGTYNEQFF | 1 | TRBV3- | TRBJ2-1 |
| (SEQ ID NO: 176) | | 1 | |
| CASCFTSLGTGELFF | 1 | TRBV27 | TRBJ2-2 |
| (SEQ ID NO: 177) | | | |
| CASSQGDRDYEQFF | 1 | TRBV3- | TRBJ2-1 |
| (SEQ ID NO: 178) | | 1 | |
| CASGSSEQFF | 1 | TRBV9- | TRBJ2-1 |
| (SEQ ID NO: 179) | | 1 | |
| CASSSGTAQSEKLFF | 1 | TRBV7- | TRBJ1-4 |
| (SEQ ID NO: 180) | | 9 | |
| CASGTSQAYEQYF | 1 | TRBV28 | TRBJ2-7 |
| (SEQ ID NO: 181) | | | |
| CASSSLGTEVYEQYF | 1 | TRBV7- | TRBJ2-7 |
| (SEQ ID NO: 182) | | 2 | |
| CASIQGHEQYF | 1 | TRBV10- | TRBJ2-7 |
| (SEQ ID NO: 183) | | 2 | |
| CASSSPGDSYEQYF | 1 | TRBV6- | TRBJ2-7 |
| (SEQ ID NO: 184) | | 5 | |
| CASNAGAGFGYEQYF | 1 | TRBV28 | TRBJ2-7 |
| (SEQ ID NO: 185) | | | |
| CASSSSGRAQTDTQYF | 1 | TRBV9 | TRBJ2-3 |
| (SEQ ID NO: 186) | | | |
| CASRGTVRGGYEQYF | 1 | TRBV12- | TRBJ2-7 |
| (SEQ ID NO: 187) | | 3 | |
| CASSSTGGISWNTEAFF | 1 | TRBV5- | TRBJ1-1 |
| (SEQ ID NO: 188) | | 4 | |
| CASRLGTAPAFF | 1 | TRBV13 | TRBJ1-1 |
| (SEQ ID NO: 189) | | | |
| CASSSTGPPFNYGYTF | 1 | TRBV7- | TRBJ1-2 |
| (SEQ ID NO: 190) | | 9 | |
| CASRRTAATNEKLFF | 1 | TRBV2 | TRBJ1-4 |
| (SEQ ID NO: 191) | | | |
| CASSSTPGGLWYGYTF | 1 | TRBV27 | TRBV1-2 |
| (SEQ ID NO: 192) | | | |
| CASSARNSNQPQHF | 1 | TRBV28 | TRBJ1-5 |
| (SEQ ID NO: 193) | | | |
| CASSSTYEQYF | 1 | TRBV7- | TRBJ2-7 |
| (SEQ ID NO: 194) | | 9 | |
| CASSARTGAYGYTF | 1 | TRBV3- | TRBJ1-2 |
| (SEQ ID NO: 195) | | 1 | |
| CASSTRTTHTYSNQPQHF | 1 | TRBV7- | TRBJ1-5 |
| (SEQ ID NO: 196) | | 9 | |
| CASSDEAGYGYTF | 1 | TRBV6- | TRBJ1-2 |
| (SEQ ID NO: 197) | | 1 | |
| CASSVKRLNTEAFF | 1 | TRBV7- | TRBJ1-1 |
| (SEQ ID NO: 198) | | 9 | |
| CASSDLPSGAPQETQYF | 1 | TRBV2 | TRBJ2-5 |
| (SEQ ID NO: 199) | | | |
| CASSYGQSFEQYF | 1 | TRBV5- | TRBJ2-7 |
| (SEQ ID NO: 200) | | 1 | |
| CASSETASSTDTQYF | 1 | TRBV2 | TRBJ2-3 |
| (SEQ ID NO: 201) | | | |
| CASSYKQGIHEQYF | 1 | TRBV6- | TRBV2-7 |
| (SEQ ID NO: 202) | | 5 | |
| CASSFAETQYF | 1 | TRBV27 | TRBJ2-5 |
| (SEQ ID NO: 203) | | | |
| CASSYSQGNYGYTF | 1 | TRBV11- | TRBV1-2 |
| (SEQ ID NO: 204) | | 2 | |
| CASSFDPRGEKLFF | 1 | TRBV27 | TRBJ1-4 |
| (SEQ ID NO: 205) | | | |
| CASSYTPGGNTDTQYF | 1 | TRBV6- | TRBJ2-3 |
| (SEQ ID NO: 206) | | 6 | |
| CASSFGTGSSETQYF | 1 | TRBV7- | TRBJ2-5 |
| (SEQ ID NO: 207) | | 8 | |
| CASSYWAGDYGYTF | 1 | TRBV6- | TRBJ1-2 |
| (SEQ ID NO: 208) | | 5 | |
| CASSFGVYNEQFF | 1 | TRBV12- | TRBJ2-1 |
| (SEQ ID NO: 209) | | 4 | |
| CASTAGFNQPQHF | 1 | TRBV6- | TRBJ1-5 |
| (SEQ ID NO: 210) | | 1 | |
| CASSFPPSGDTDTQYF | 1 | TRBV7- | TRBJ2-3 |
| (SEQ ID NO: 211) | | 8 | |
| CASTDVTSGQETQYF | 1 | TRBV5- | TRBJ2-5 |
| (SEQ ID NO: 212) | | 1 | |
| CASSFSGSDTGELFF | 1 | TRBV19 | TRBJ2-2 |
| (SEQ ID NO: 213) | | | |
| CASTGIAGPTDTQYF | 1 | TRBV27 | TRBJ2-3 |
| (SEQ ID NO: 214) | | | |
| CASSFTGTPTYEQYF | 1 | TRBV9 | TRBJ2-7 |
| (SEQ ID NO: 215) | | | |
| CATIGPAGDTQYF | 1 | TRBV12- | TRBJ2-3 |
| (SEQ ID NO: 216) | | 3 | |
| CASSHGQGNQPQHF | 1 | TRBV13 | TRBJ1-5 |
| (SEQ ID NO: 217) | | | |
| CATPSGNTIYF | 1 | TRBV19 | TRBJ1-3 |
| (SEQ ID NO: 218) | | | |
| CASSHPTRSYNEQFF | 1 | TRBV3- | TRBJ2-1 |
| (SEQ ID NO: 219) | | 1 | |
| CATSDLGLGVNEQFF | 1 | TRBV24 | TRBJ2-1 |
| (SEQ ID NO: 220) | | | |
| CASSHRRGIPPPPLYNEQFF | 1 | TRBV4- | TRBJ2-7 |
| (SEQ ID NO: 221) | | 2/3 | |
| CATSRAQGQPQHF | 1 | TRBV15 | TRBJ1-5 |
| (SEQ ID NO: 222) | | | |
| CASSIAARAGELFF | 1 | TRBV19 | TRBJ2-2 |
| (SEQ ID NO: 223) | | | |
| CATSRDHSSGASQGNIQYF | 1 | TRBV15 | TRBJ2-4 |
| (SEQ ID NO: 224) | | | |
| CASSIGVLNTEAFF | 1 | TRBV19 | TRBJ1-1 |
| (SEQ ID NO: 225) | | | |
| CATSRDNTGYTGELFF | 1 | TRBV15 | TRBJ2-2 |
| (SEQ ID NO: 226) | | | |
| CASSKLDSGYTF | 1 | TRBV28 | TRBJ1-2 |
| (SEQ ID NO: 227) | | | |
| CATSRDQGSDTQYF | 1 | TRBV15 | TRBJ2-4 |
| (SEQ ID NO: 228) | | | |
| CASSKPPETQYF | 1 | TRBV28 | TRBJ2-5 |
| (SEQ ID NO: 229) | | | |
| CAWDSTGISYNEQFF | 1 | TRBV30 | TRBJ2-1 |
| (SEQ ID NO: 230) | | | |
| CASSLAGGIAKNIQYF | 1 | TRBV12- | TRBJ2-4 |
| (SEQ ID NO: 231) | | 3 | |
| CAWRATEGQETQYF | 1 | TRBV30 | TRBJ2-5 |
| (SEQ ID NO: 232) | | | |
| CASSLAPWMDYGYTF | 1 | TRBV7- | TRBJ1-2 |
| (SEQ ID NO: 233) | | 9 | |
| CAWSDSGSSYEQYF | 1 | TRBV30 | TRBJ2-7 |
| (SEQ ID NO: 234) | | | |
| CASSLFTGGTYEQYF | 1 | TRBV7- | TRBJ2-7 |
| (SEQ ID NO: 235) | | 6 | |
| CAWSVTGTRLYEQYF | 1 | TRBV30 | TRBJ2-7 |
| (SEQ ID NO: 236) | | | |
| CASSLGGLELGFEQFF | 1 | TRBV5- | TRBJ2-1 |
| (SEQ ID NO: 237) | | 4 | |
| CSALDSYSNQPQHF | 1 | TRBV20- | TRBJ1-5 |
| (SEQ ID NO: 238) | | 1 | |
| CASSLGGSNQPQHF | 1 | TRBV27 | TRBJ1-5 |
| (SEQ ID NO: 239) | | | |
| CSARAAIGTMNTEAFF | 1 | TRBV20- | TRBJ1-1 |
| (SEQ ID NO: 240) | | 1 | |
| CASSLGLGTGELFF | 1 | TRBV13 | TRBJ2-2 |
| (SEQ ID NO: 241) | | | |
| CSARDADGYESEKLFF | 1 | TRBV20- | TRBJ1-4 |
| (SEQ ID NO: 242) | | 1 | |
| CASSLGRVEAFF | 1 | TRBV7- | TRBJ1-1 |
| (SEQ ID NO: 243) | | 9 | |
| CSARDRQGYSNQPQHF | 1 | TRBV20- | TRBJ1-5 |
| (SEQ ID NO: 244) | | 1 | |
| CASSLGTSQETQYF | 1 | TRBV7- | TRBJ2-5 |
| (SEQ ID NO: 245) | | 8 | |
| CSARPPNGRYNEQFF | 1 | TRBV20- | TRBJ2-1 |
| (SEQ ID NO: 246) | | 1 | |
| CASSLLRLAGETTYNEQFF | 1 | TRBV28 | TRBJ2-1 |
| (SEQ ID NO: 247) | | | |
| CSARVKGLAGIRSYEQYF | 1 | TRBV20- | TRBJ2-7 |
| (SEQ ID NO: 248) | | 1 | |
| CASSLPGTGTSPLHF | 1 | TRBV28 | TRBJ1-6 |
| (SEQ ID NO: 249) | | | |
| CSDTVRRGPGGYTF | 1 | TRBV20- | TRBJ1-2 |
| (SEQ ID NO: 250) | | 1 | |
| CASSLRGYTDTQYF | 1 | TRBV27 | TRBJ2-3 |
| (SEQ ID NO: 251) | | | |
| CSVLGQGPSYEQYF | 1 | TRBV29- | TRBJ2-7 |
| (SEQ ID NO: 252) | | 1 | |
| CASSLRQGGYEQYF | 1 | TRBV10- | TRBJ2-7 |
| (SEQ ID NO: 253) | | 2 | |
| CSVVEGGEQYF | 1 | TRBV29- | TRBJ2-7 |
| (SEQ ID NO: 254) | | 1 | |

Table 3c
Patient 2, Challenge 2
PB-IE CD8 TCRβ (75)

| | | | |
|---|---|---|---|
| CAWSGVSADTQYF | 4 | TRBV30- | TRBJ2-3 |
| (SEQ ID NO: 255) | | 1 | |
| CASSLSGWTEAFF | 1 | TRBV27 | TRBJ1-1 |
| (SEQ ID NO: 256) | | | |
| CASSLGARTGELFF | 2 | TRBV28 | TRBJ2-2 |
| (SEQ ID NO: 257) | | | |
| CASSLSQGGHNEQFF | 1 | TRBV7- | TRBJ2-1 |
| (SEQ ID NO: 148) | | 6 | |
| CASSPRSRTDTQYF | 2 | TRBV27 | TRBJ2-3 |
| (SEQ ID NO: 258) | | | |
| CASSLTASNQPQHF | 1 | TRBV6- | TRBJ1-5 |
| (SEQ ID NO: 259) | | 5 | |
| CASSVGGVQPQHF | 2 | TRBV12- | TRBJ1-5 |
| (SEQ ID NO: 151) | | 4 | |
| CASSPAGYNEQFF | 1 | TRBV28 | TRBJ2-1 |
| (SEQ ID NO: 260) | | | |
| CSAREGDTQYF | 2 | TRBV20- | TRBJ2-3 |
| (SEQ ID NO: 261) | | 1 | |
| CASSPGLVVDEQYF | 1 | TRBV27 | TRBJ2-7 |
| (SEQ ID NO: 262) | | | |
| CASSSGELFF | 1 | TRBV28 | TRBJ2-2 |
| (SEQ ID NO: 263) | | | |
| CASSPPGQGITDTQYF | 1 | TRBV12- | TRBJ2-7 |
| (SEQ ID NO: 264) | | 3/4 | |
| CASDRGETQYF | 1 | TRBV28 | TRBJ2-5 |
| (SEQ ID NO: 265) | | | |
| CASSPRNKRKADQPQHF | 1 | TRBV18 | TRBJ1-5 |
| (SEQ ID NO: 266) | | | |
| CASGESNEQYF | 1 | TRBV6- | TRBJ2-7 |
| (SEQ ID NO: 267) | | 1 | |
| CASSPRVDRGLHEQYF | 1 | TRBV6- | TRBJ2-7 |
| (SEQ ID NO: 268) | | 6 | |
| CASKPLQGYNEQFF | 1 | TRBV6- | TRBJ2-1 |
| (SEQ ID NO: 269) | | 5 | |
| CASSPTSGRTTSYEQYF | 1 | TRBV4- | TRBJ2-7 |
| (SEQ ID NO: 174) | | 1 | |
| CASNIRASNQPQHF | 1 | TRBV28 | TRBJ1-5 |
| (SEQ ID NO: 270) | | | |
| CASSPWDGSSYEQYF | 1 | TRBV19 | TRBJ2-7 |
| (SEQ ID NO: 271) | | | |
| CASNLAGGSNEQFF | 1 | TRBV6- | TRBJ2-1 |
| (SEQ ID NO: 153) | | 5 | |
| CASSQDGGDYGYTF | 1 | TRBV4- | TRBJ1-2 |
| (SEQ ID NO: 272) | | 2 | |
| CASQRQSYEQYF | 1 | TRBV5- | TRBJ2-7 |
| (SEQ ID NO: 273) | | 6 | |
| CASSQDGGTYNEQFF | 1 | TRBV3- | TRBJ2-1 |
| (SEQ ID NO: 176) | | 1 | |
| CASRFGQGGNSNQPQHF | 1 | TRBV12- | TRBJ1-5 |
| (SEQ ID NO: 274) | | 3 | |
| CASSQGRGKVYEQYF | 1 | TRBV4- | TRBJ2-7 |
| (SEQ ID NO: 275) | | 1 | |
| CASRSRGTIYEQYF | 1 | TRBV7- | TRBJ2-7 |
| (SEQ ID NO: 276) | | 9 | |
| CASSQTGLTNYGYTF | 1 | TRBV6- | TRBJ1-2 |
| (SEQ ID NO: 277) | | 1 | |
| CASSDGLAYEQYF | 1 | TRBV19 | TRBJ2-7 |
| (SEQ ID NO: 278) | | | |
| CASSRGVEQYF | 1 | TRBV27 | TRBJ2-7 |
| (SEQ ID NO: 279) | | | |
| CASSDTGSINQPQHF | 1 | TRBV2 | TRBJ1-5 |
| (SEQ ID NO: 280) | | | |
| CASSRSGNTEAFF | 1 | TRBV28 | TRBJ1-1 |
| (SEQ ID NO: 281) | | | |
| CASSFASSSGNTIYF | 1 | TRBV28 | TRBJ1-3 |
| (SEQ ID NO: 282) | | | |
| CASSSPLGGYGYTF | 1 | TRBV12- | TRBJ1-2 |
| (SEQ ID NO: 283) | | 3/4 | |
| CASSFIVLSGSSYEQYF | 1 | TRBV28 | TRBJ2-7 |
| (SEQ ID NO: 284) | | | |
| CASSSPVRSGANVLTF | 1 | TRBV7- | TRBJ2-6 |
| (SEQ ID NO: 285) | | 9 | |
| CASSFSGRTYEQYF | 1 | TRBV27 | TRBJ2-7 |
| (SEQ ID NO: 286) | | | |
| CASSSTPGGLWYGYTF | 1 | TRBV27 | TRBJ1-2 |
| (SEQ ID NO: 192) | | | |
| CASSFSQVDEQFF | 1 | TRBV12- | TRBJ2-1 |
| (SEQ ID NO: 287) | | 3 | |
| CASSWMTRIYNEQFF | 1 | TRBV5- | TRBJ2-1 |
| (SEQ ID NO: 288) | | 5 | |
| CASSGQQGGSYGYTF | 1 | TRBV10- | TRBJ1-2 |
| (SEQ ID NO: 289) | | 2 | |
| CASSYDSAYEQYF | 1 | TRBV27 | TRBJ2-7 |
| (SEQ ID NO: 290) | | | |
| CASSGSATGELFF | 1 | TRBV7- | TRBJ2-2 |
| (SEQ ID NO: 291) | | 6 | |
| CASSYDVRSGNYEQYF | 1 | TRBV6- | TRBJ2-7 |
| (SEQ ID NO: 145) | | 6 | |
| CASSKLDSGYTF | 1 | TRBV28 | TRBJ1-2 |
| (SEQ ID NO: 227) | | | |
| CASSYSVAGAFF | 1 | TRBV6- | TRBJ1-1 |
| (SEQ ID NO: 292) | | 2 | |
| CASSLDDGYTF | 1 | TRBV7- | TRBJ1-2 |
| (SEQ ID NO: 293) | | 8 | |
| CASTAGFNQPQHF | 1 | TRBV6- | TRBJ1-5 |
| (SEQ ID NO: 210) | | 1 | |
| CASSLDRGDTQYF | 1 | TRBV7- | TRBJ2-3 |
| (SEQ ID NO: 294) | | 6 | |
| CASTFSYAGTDTQYF | 1 | TRBV5- | TRBJ2-3 |
| (SEQ ID NO: 295) | | 4 | |
| CASSLEQSAMNTEAFF | 1 | TRBV11- | TRBJ1-1 |
| (SEQ ID NO: 296) | | 2 | |
| CATSTLQGGPRDEQFF | 1 | TRBV15 | TRBJ2-1 |
| (SEQ ID NO: 297) | | | |
| CASSLGGELFF | 1 | TRBV27 | TRBJ2-2 |
| (SEQ ID NO: 298) | | | |
| CATSVTGSYGYTF | 1 | TRBV15 | TRBJ1-2 |
| (SEQ ID NO: 299) | | | |
| CASSLGGSGSYEQYF | 1 | TRBV11- | TRBJ2-7 |
| (SEQ ID NO: 300) | | 2 | |
| CATTGSSYEQYF | 1 | TRBV2 | TRBJ2-7 |
| (SEQ ID NO: 301) | | | |
| CASSLGNTEAFF | 1 | TRBV7- | TRBJ1-1 |
| (SEQ ID NO: 302) | | 9 | |
| CAWNRGGSSPLHF | 1 | TRBV30 | TRBJ1-6 |
| (SEQ ID NO: 303) | | | |
| CASSLGQGAQTQYF | 1 | TRBV27 | TRBJ2-5 |
| (SEQ ID NO: 304) | | | |
| CAWRRQGEEKLFF | 1 | TRBV30 | TRBJ1-4 |
| (SEQ ID NO: 305) | | | |
| CASSLGRVEAFF | 1 | TRBV7- | TRBJ1-1 |
| (SEQ ID NO: 243) | | 9 | |
| CAYSPGKNTEAFF | 1 | TRBV30 | TRBJ1-1 |
| (SEQ ID NO: 306) | | | |
| CASSLLGGVQETQYF | 1 | TRBV9 | TRBJ2-5 |
| (SEQ ID NO: 307) | | | |
| CSAPGQRNTIYF | 1 | TRBV20- | TRBJ1-3 |
| (SEQ ID NO: 308) | | 1 | |
| CASSLRGTSSYNSPLHF | 1 | TRBV12- | TRBJ1-5 |
| (SEQ ID NO: 309) | | 3/4 | |
| CSARDPDSPPGGYTF | 1 | TRBV20- | TRBJ1-2 |
| (SEQ ID NO: 310) | | 1 | |
| CASSLSGGSWTEAFF | 1 | TRBV27 | TRBJ1-1 |
| (SEQ ID NO: 311) | | | |
| CSVGGGEDYTF | 1 | TRBV29- | TRBJ1-2 |
| (SEQ ID NO: 312) | | 1 | |

Patient 3
PB-IE CD8 TCRβ (67)

| | | | |
|---|---|---|---|
| CASSQERGGKWAYEQYF | 11 | TRBV4- | TRBJ2-7 |
| (SEQ ID NO: 313) | | 3 | |
| CASSLPVAGGQETQYF | 1 | TRBV11- | TRBJ2-5 |
| (SEQ ID NO: 314) | | 2 | |
| CATSDLLTGNAFF | 11 | TRBV24 | TRBJ101 |
| (SEQ ID NO: 315) | | | |
| CASSLTGGSYGYTF | 1 | TRBV7- | TRBJ1-2 |
| (SEQ ID NO: 316) | | 9 | |
| CASSLNGGSYEQYF | 4 | TRBV5- | TRBJ2-7 |
| (SEQ ID NO: 317) | | 1 | |
| CASSLVWNTDTQYF | 1 | TRBV7- | TRBJ2-3 |
| (SEQ ID NO: 318) | | 8 | |
| CASRPIPGQEKSSGANVLTF | 2 | TRBV3- | TRBJ2-6 |
| (SEQ ID NO: 319) | | 1 | |
| CASSPDGTGIEQFF | 1 | TRBV5- | TRBJ2-1 |
| (SEQ ID NO: 320) | | 8 | |
| CASSFFPRTGSNEQFF | 2 | TRBV27 | TRBJ2-1 |
| (SEQ ID NO: 321) | | | |
| CASSPGAFTNTEAFF | 1 | TRBV4- | TRBJ1-1 |
| (SEQ ID NO: 322) | | 3 | |
| CASSQEGVGGNYGYTF | 2 | TRBV4- | TRBJ1-2 |
| (SEQ ID NO: 323) | | 3 | |
| CASSPWGSYEQYF | 1 | TRBV11- | TRBJ2-7 |
| (SEQ ID NO: 324) | | 3 | |
| CASSQEQGTNYGYTF | 2 | TRBV4- | TRBJ1-2 |
| (SEQ ID NO: 325) | | 3 | |
| CASSPYIGEVGNTEAFF | 1 | TRBV7- | TRBJ1-1 |
| (SEQ ID NO: 326) | | 8 | |
| CASSQLTLGPAKNIQYF | 2 | TRBV4- | TRBJ2-4 |
| (SEQ ID NO: 327) | | 2 | |
| CASSQEESADTQYF | 1 | TRBV4- | TRBJ2-3 |
| (SEQ ID NO: 328) | | 3 | |
| CASSQQLNYNSPLHF | 2 | TRBV4- | TRBJ1-6 |
| (SEQ ID NO: 329) | | 2 | |
| CASSQELGQSSYNSPLHF | 1 | TRBV4- | TRBJ1-6 |
| (SEQ ID NO: 330) | | 2 | |
| CASSVEGGGGPSTDTQYF | 2 | TRBV9 | TRBJ2-3 |
| (SEQ ID NO: 331) | | | |
| CASSQERGTAYGYTF | 1 | TRBV4- | TRBJ1-2 |
| (SEQ ID NO: 332) | | 2 | |
| CASSVPKGGFNEQFF | 2 | TRBV11- | TRBJ2-1 |
| (SEQ ID NO: 333) | | 2 | |
| CASSQERSADTQYF | 1 | TRBV4- | TRBJ2-3 |
| (SEQ ID NO: 334) | | 3 | |
| CASGSGVTEAFF | 1 | TRBV7- | TRBJ1-1 |
| (SEQ ID NO: 335) | | 8 | |
| CASSQPQGWGNTEAFF | 1 | TRBV4- | TRBJ1-1 |
| (SEQ ID NO: 336) | | 1 | |
| CASRPGATNYGYTF | 1 | TRBV4- | TRBV1-2 |
| (SEQ ID NO: 337) | | 2 | |
| CASSSYTGELFF | 1 | TRBV7- | TRBJ2-2 |
| (SEQ ID NO: 338) | | 9 | |
| CASSLDGGVNGYTF | 1 | TRBV5- | TRBJ1-2 |
| (SEQ ID NO: 339) | | 1 | |
| CASSYIQGNQPQHF | 1 | TRBV5- | TRBJ1-5 |
| (SEQ ID NO: 340) | | 8 | |
| CASSLDGVQIYGYTF | 1 | TRBV7- | TRBJ1-2 |
| (SEQ ID NO: 341) | | 9 | |
| CASSYQGGGTDTQYF | 1 | TRBV6- | TRBJ2-3 |
| (SEQ ID NO: 342) | | 6 | |
| CASSLGNTEAFF | 1 | TRBV7- | TRBJ1-1 |
| (SEQ ID NO: 302) | | 9 | |
| CASTRTGTGPATNEKLFF | 1 | TRBV6- | TRBJ1-4 |
| (SEQ ID NO: 343) | | 5 | |
| CASSLGQGVGTEAFF | 1 | TRBV27 | TRBJ1-1 |
| (SEQ ID NO: 344) | | | |
| CSASLQENTEAFF | 1 | TRBV20- | TRBJ1-1 |
| (SEQ ID NO: 345) | | 1 | |
| CASSLGYEQYF | 1 | TRBV11- | TRBJ2-7 |
| (SEQ ID NO: 346) | | 2 | |
| CSVQERDSANYGYTF | 1 | TRBV29- | TRBJ1-2 |
| (SEQ ID NO: 347) | | 1 | |

Table 3d
Patient 4
PB-IE CD8 TCRβ (120)

| | | | |
|---|---|---|---|
| CASRTGNQPQHF | 17 | TRBV7- | TRBJ1-5 |
| (SEQ ID NO: 348) | | 9 | |
| CASSLGSNEQFF | 1 | TRBV12- | TRBJ2-1 |
| (SEQ ID NO: 349) | | 3 | |
| CASRGGNTEAFF | 8 | TRBV7- | TRBJ1-1 |
| (SEQ ID NO: 350) | | 9 | |
| CASSLGSRETQYF | 1 | TRBV28 | TRBJ2-5 |
| (SEQ ID NO: 351) | | | |
| CASSFRVGYNEQFF | 8 | TRBV13 | TRBJ2-1 |
| (SEQ ID NO: 352) | | | |
| CASSLGTSEQYF | 1 | TRBV7- | TRBJ2-7 |
| (SEQ ID NO: 353) | | 8 | |
| CASIAGNTEAFF | 5 | TRBV7- | TRBJ1-1 |
| (SEQ ID NO: 354) | | 9 | |
| CASSLGTTSNEQYF | 1 | TRBV27 | TRBJ2-7 |
| (SEQ ID NO: 355) | | | |
| CASSSSYEQYF | 5 | TRBV6- | TRBJ2-7 |
| (SEQ ID NO: 356) | | 5 | |
| CASSLLNTEAFF | 1 | TRBV11- | TRBJ1-1 |
| (SEQ ID NO: 357) | | 2 | |
| CASSLGNRPEAFF | 4 | TRBV7- | TRBJ1-1 |
| (SEQ ID NO: 358) | | 9 | |
| CASSLSRDNYNEQFF | 1 | TRBV11- | TRBJ2-1 |
| (SEQ ID NO: 359) | | 1 | |
| CASSFGSDTQYF | 3 | TRBV7- | TRBV2-3 |
| (SEQ ID NO: 360) | | 8 | |
| CASSLVGVADTQYF | 1 | TRBV7- | TRBJ2-3 |
| (SEQ ID NO: 361) | | 9 | |
| CAWRSGGASPLHF | 3 | TRBV30 | TRBJ1-6 |
| (SEQ ID NO: 362) | | | |
| CASSPEPTGSNEQFF | 1 | TRBV7- | TRBJ2-1 |
| (SEQ ID NO: 363) | | 6 | |
| CASKAGEFEFF | 2 | TRBV6- | TRBJ2-1 |
| (SEQ ID NO: 364) | | 5 | |
| CASSPGQGVTEAFF | 1 | TRBV19 | TRBJ1-1 |
| (SEQ ID NO: 365) | | | |
| CASSLEYEQYF | 2 | TRBV12- | TRBJ2-7 |
| (SEQ ID NO: 366) | | 3/4 | |
| CASSPGTVYGYTF | 1 | TRBV7- | TRBJ1-2 |
| (SEQ ID NO: 367) | | 8 | |
| CASSRLGGRAGETQYF | 2 | TRBV28 | TRBJ2-5 |
| (SEQ ID NO: 368) | | | |
| CASSPQGVGADYGYTF | 1 | TRBV7- | TRBJ1-2 |
| (SEQ ID NO: 369) | | 9 | |
| CATTGGLGTEAFF | 2 | TRBV2 | TRBJ1-1 |
| (SEQ ID NO: 370) | | | |
| CASSPRAEDWTYYGYTF | 1 | TRBV9 | TRBJ1-2 |
| (SEQ ID NO: 371) | | | |
| CAGARGNQPQHF | 1 | TRBV7- | TRBJ1-5 |
| (SEQ ID NO: 372) | | 9 | |
| CASSQDGISGSGEQYF | 1 | TRBV4- | TRBJ2-7 |
| (SEQ ID NO: 373) | | 3 | |
| CAGTSGNQPQHF | 1 | TRBV7- | TRBJ1-5 |
| (SEQ ID NO: 374) | | 9 | |
| CASSQGATSNQPQHF | 1 | TRBV4- | TRBJ1-5 |
| (SEQ ID NO: 375) | | 2 | |
| CASEPLAGTNEQFF | 1 | TRBV18 | TRBJ2-1 |
| (SEQ ID NO: 376) | | | |
| CASSRTSGGTGETQYF | 1 | TRBV6- | TRBJ2-5 |
| (SEQ ID NO: 377) | | 2 | |
| CASGQDWSSYNEQFF | 1 | TRBV6- | TRBJ2-1 |
| (SEQ ID NO: 378) | | 1 | |
| CASSSDSHYSNQPQHF | 1 | TRBV7- | TRBJ1-5 |
| (SEQ ID NO: 379) | | 8 | |
| CASMTGNSNQPQHF | 1 | TRBV7- | TRBJ1-5 |
| (SEQ ID NO: 380) | | | |
| CASSSLGGRAGETQYF | 1 | TRBV289 | TRBJ2-5 |
| (SEQ ID NO: 381) | | | |
| CASRPANQETQYF | 1 | TRBV11- | TRBJ2-5 |
| (SEQ ID NO: 382) | | 2 | |
| CASSTERLGDGYTF | 1 | TRBV12- | TRBJ1-2 |
| (SEQ ID NO: 383) | | 3/4 | |
| CASRSMDTYEQYF | 1 | TRBV5- | TRBJ2-7 |
| (SEQ ID NO: 384) | | 1 | |
| CASSTGNQETQYF | 1 | TRBV7- | TRBJ2-5 |
| (SEQ ID NO: 385) | | 9 | |
| CASRTGTFFNQPQHF | 1 | TRBV12- | TRBJ1-5 |
| (SEQ ID NO: 386) | | 3/4 | |
| CASSTGNQPQHF | 1 | TRBV7- | TRBJ1-5 |
| (SEQ ID NO: 387) | | 9 | |
| CASSAPEGGGFTDTQYF | 1 | TRBV9 | TRBJ2-3 |
| (SEQ ID NO: 388) | | | |
| CASSTRQGTTNTGELFF | 1 | TRBV4- | TRBJ2-2 |
| (SEQ ID NO: 389) | | 2 | |
| CASSATTGFSEKLFF | 1 | TRBV7- | TRBJ1-4 |
| (SEQ ID NO: 390) | | 6 | |
| CASSTTGYGELFF | 1 | TRBV6- | TRBJ2-2 |
| (SEQ ID NO: 391) | | 2 | |
| CASSFGTAGTQYF | 1 | TRBV28 | TRBJ2-5 |
| (SEQ ID NO: 392) | | | |
| CASSWGTDYEQYF | 1 | TRBV7- | TRBJ2-7 |
| (SEQ ID NO: 393) | | 8 | |
| CASSGGNQPQHF | 1 | TRBV7- | TRBJ1-5 |
| (SEQ ID NO: 394) | | 9 | |
| CASSYGTLDPYGYTF | 1 | TRBV12- | TRBJ1-2 |
| (SEQ ID NO: 395) | | 3/4 | |
| CASSGQATSYEQYF | 1 | TRBV2 | TRBJ2-7 |
| (SEQ ID NO: 396) | | | |
| CASSYSANNYGYTF | 1 | TRBV6- | TRBJ1-2 |
| (SEQ ID NO: 397) | | 5 | |
| CASSHSRDRVGEKLFF | 1 | TRBV18 | TRBJ1-4 |
| (SEQ ID NO: 398) | | | |
| CASSYTGLEQYF | 1 | TRBV4- | TRBJ2-7 |
| (SEQ ID NO: 399) | | 3 | |
| CASSIAEGTIYNEQFF | 1 | TRBV19 | TRBJ2-1 |
| (SEQ ID NO: 400) | | | |
| CASSYVGGAEAFF | 1 | TRBV6- | TRBJ1-1 |
| (SEQ ID NO: 401) | | 2 | |
| CASSIPGRRETQYF | 1 | TRBV6- | TRBJ2-5 |
| (SEQ ID NO: 402) | | 2 | |
| CASSYVREDYGYTF | 1 | TRBV6- | TRBJ1-2 |
| (SEQ ID NO: 403) | | 5 | |
| CASSISSDGYTF | 1 | TRBV7- | TRBJ1-2 |
| (SEQ ID NO: 404) | | 9 | |
| CASTGGYNSPLHF | 1 | TRBV27 | TRBJ1-6 |
| (SEQ ID NO: 405) | | | |
| CASSLDEGYTGELFF | 1 | TRBV7- | TRBJ2-2 |
| (SEQ ID NO: 406) | | 9 | |
| CASTLGGEQYF | 1 | TRBV28 | TRBJ2-7 |
| (SEQ ID NO: 407) | | | |
| CASSLDSSNQPQHF | 1 | TRBV7- | TRBJ1-5 |
| (SEQ ID NO: 408) | | 9 | |
| CASVQGNTEAFF | 1 | TRBV7- | TRBJ1-1 |
| (SEQ ID NO: 409) | | 9 | |
| CASSLGAGANVLTF | 1 | TRBV27 | TRBJ2-6 |
| (SEQ ID NO: 410) | | | |
| CATSTYEGADQPQHF | 1 | TRBV15 | TRBJ1-5 |
| (SEQ ID NO: 411) | | | |
| CASSLGGLAGEQYF | 1 | TRBV7- | TRBJ2-7 |
| (SEQ ID NO: 412) | | 8 | |
| CAWSVGGIQPQHF | 1 | TRBV30 | TRBJ1-5 |
| (SEQ ID NO: 413) | | | |
| CASSLGGQLFF | 1 | TRBV7- | TRBJ1-4 |
| (SEQ ID NO: 414) | | 8 | |
| CSAPGHLNYGYTF | 1 | TRBV20- | TRBJ1-2 |
| (SEQ ID NO: 415) | | 1 | |
| CASSLGLAGEQYF | 1 | TRBV28 | TRBJ2-7 |
| (SEQ ID NO: 416) | | | |
| CSARDGGGDWEKLFF | 1 | TRBV20- | TRBJ1-4 |
| (SEQ ID NO: 417) | | 1 | |
| CASSLGSKSTQYF | 1 | TRBV28 | TRBJ2-3 |
| (SEQ ID NO: 418) | | | |

Patient 5
PB-IE CD8 TCRβ (51)

| | | | |
|---|---|---|---|
| CSAREGQFSGNTIYF | 8 | TRBV20- | TRBJ1-3 |
| (SEQ ID NO: 419) | | 1 | |
| CASSNPGLQETQYF | 1 | TRBV7- | TRBJ2-5 |
| (SEQ ID NO: 420) | | 2 | |
| CASSYFGGPGNTIYF | 2 | TRBV6- | TRBJ1-3 |
| (SEQ ID NO: 421) | | 5 | |
| CASSNDRARAKNIQYF | 1 | TRBV27 | TRBJ2-4 |
| (SEQ ID NO: 422) | | | |
| CASSQDRRSSYNSPLHF | 2 | TRBV4- | TRBJ1-6 |
| (SEQ ID NO: 423) | | 3 | |
| CASSLVQDVGDEAFF | 1 | TRBV5- | TRBJ1-1 |
| (SEQ ID NO: 424) | | 1 | |
| CASSQDLQTTFYEQYF | 2 | TRBV4- | TRBJ2-7 |
| (SEQ ID NO: 425) | | 2 | |
| CASSLTSGPLYEQFF | 1 | TRBV12- | TRBJ2-1 |
| (SEQ ID NO: 426) | | 3/4 | |
| CASSAVGGAYEQYF | 2 | TRBV7- | TRBJ2-7 |
| (SEQ ID NO: 427) | | 3/4 | |
| CASSLRGGGETQYF | 1 | TRBV28 | TRBJ2-5 |
| (SEQ ID NO: 428) | | | |
| CSVTTGGQEAFF | 1 | TRBV29 | TRBJ1-1 |
| (SEQ ID NO: 429) | | | |
| CASSLNLGNTEAFF | 1 | TRBV7- | TRBJ1-1 |
| (SEQ ID NO: 430) | | 6 | |
| CSVALGAVRSTDTQYF | 1 | TRBV29 | TRBJ2-3 |
| (SEQ ID NO: 431) | | | |
| CASSLGETQYF | 1 | TRBV7- | TRBJ2-5 |
| (SEQ ID NO: 432) | | 2 | |
| CSARDIVNANTGELFF | 1 | TRBV20- | TRBJ2-2 |
| (SEQ ID NO: 433) | | 1 | |
| CASSLFMGWEQYF | 1 | TRBV27 | TRBJ2-7 |
| (SEQ ID NO: 434) | | | |
| CSAPIQAGTEAFF | 1 | TRBV29 | TRBJ1-1 |
| (SEQ ID NO: 435) | | | |
| CASSLEAGTLDYGYTF | 1 | TRBV5- | TRBJ1-2 |
| (SEQ ID NO: 436) | | 1 | |
| CATSRDRSWDSPLHF | 1 | TRBV15 | TRBJ1-6 |
| (SEQ ID NO: 437) | | | |
| CASSIGTGGPYEQYF | 1 | TRBV7- | TRBJ2-7 |
| (SEQ ID NO: 438) | | 7 | |
| CASTNGGMNTEAFF | 1 | TRBV7- | TRBJ1-1 |
| (SEQ ID NO: 439) | | 9 | |
| CASSHPSSPHEKLFF | 1 | TRBV4- | TRBJ1-4 |
| (SEQ ID NO: 440) | | 2 | |
| CASSYSVGNTGELFF | 1 | TRBV6- | TRBJ2-2 |
| (SEQ ID NO: 441) | | 5 | |
| CASSFSGGAGEQFF | 1 | TRBV7- | TRBJ2-1 |
| (SEQ ID NO: 442) | | 3/4 | |
| CASSVGVVYEQYF | 1 | TRBV9 | TRBJ2-7 |
| (SEQ ID NO: 443) | | | |
| CASSESGTGIGSQPQHF | 1 | TRBV6- | TRBJ1-5 |
| (SEQ ID NO: 444) | | 1 | |
| CASSSSTVSGNTIYF | 1 | TRBV19 | TRBJ1-3 |
| (SEQ ID NO: 445) | | | |
| CASSELTRGTDTQYF | 1 | TRBV6- | TRBJ2-3 |
| (SEQ ID NO: 446) | | 1 | |
| CASSQQAPTSSYNSPLHF | 1 | TRBV4- | TRBJ1-6 |
| (SEQ ID NO: 447) | | 1 | |
| CASSDGLAGFSTDTQYF | 1 | TRBV12- | TRBJ2-3 |
| (SEQ ID NO: 448) | | 3/4 | |
| CASSQDLSWESPLHF | 1 | TRBV4- | TRBJ1-6 |
| (SEQ ID NO: 449) | | 2 | |
| CASRLTDTQYF | 1 | TRBV19 | TRBJ2-3 |
| (SEQ ID NO: 450) | | | |
| CASSPVLGAFFGYGYTF | 1 | TRBV18 | TRBJ1-2 |
| (SEQ ID NO: 451) | | | |
| CASQQTGGFNEQYF | 1 | TRBV11- | TRBJ2-7 |
| (SEQ ID NO: 452) | | 2 | |
| CASSPTWTGGNEQFF | 1 | TRBV5- | TRBJ2-1 |
| (SEQ ID NO: 453) | | 1 | |
| CASKTGVSYNEQFF | 1 | TRBV10- | TRBJ2-1 |
| (SEQ ID NO: 454) | | 2 | |
| CASSPDRPPIYNEQFF | 1 | TRBV6- | TRBJ2-1 |
| (SEQ ID NO: 455) | | 5 | |
| CASGGGMGGQPQHF | 1 | TRBV6- | TRBJ1-5 |
| (SEQ ID NO: 456) | | 2 | |
| CASSPAVAGGRDTQYF | 1 | TRBV4- | TRBJ2-3 |
| (SEQ ID NO: 457) | | 3 | |
| CAGTTGYEQYF | 1 | TRBV30 | TRBJ2-7 |
| (SEQ ID NO: 458) | | | |

Table 3e
Patient 1, Challenge 1
PB-IE GD TCRδ (96)

| | | | |
|---|---|---|---|
| CALGGLPTLGDTPTDKLIF | 59 | DV1 | DJ1 |
| (SEQ ID NO: 459) | | | |
| CALCLLADWGYTDKLIF | 5 | DV1 | DJ1 |
| (SEQ ID NO: 460) | | | |
| CALAPLPTLGDTGPDKLIF | 2 | DV1 | DJ1 |
| (SEQ ID NO: 461) | | | |
| CALGDGGGFYTSRVLGGYAFVTTDKLIF | 2 | DV1 | DJ1 |
| (SEQ ID NO: 462) | | | |
| CALGELPYWALRGADKLIF | 2 | DV1 | DJ1 |
| (SEQ ID NO: 463) | | | |
| CALGGYADKLIF | 2 | DV2 | DJ1 |
| (SEQ ID NO: 464) | | | |
| CASSPSYGGYAVDKLIF | 2 | DV3 | DJ1 |
| (SEQ ID NO: 465) | | | |
| CACDTLLGDTLLTAQLFF | 1 | DV2 | DJ2 |
| (SEQ ID NO: 466) | | | |
| CACDTLLGENKLIF | 1 | DV2 | DJ1 |
| (SEQ ID NO: 467) | | | |
| CAFNRGLLYTDKLIF | 1 | DV3 | DJ1 |
| (SEQ ID NO: 468) | | | |
| CALGAFLPRYWGPRHTDKLIF | 1 | DV1 | DJ1 |
| (SEQ ID NO: 469) | | | |
| CALGDPSLPLNWGIRGHGIQLIF | 1 | DV1 | DJ1 |
| (SEQ ID NO: 470) | | | |
| CALGEARPSYWGIRTTDKLIF | 1 | DV1 | DJ1 |
| (SEQ ID NO: 471) | | | |
| CALGEFFPRYWGTTYTDKLIF | 1 | DV1 | DJ1 |
| (SEQ ID NO: 472) | | | |
| CALGEKPPFLSKVLGDTHYTDKLIF | 1 | DV1 | DJ1 |
| (SEQ ID NO: 473) | | | |
| CALGELIGWGPKDADKLIF | 1 | DV1 | DJ1 |
| (SEQ ID NO: 474) | | | |
| CALGELQPRYWGRRFDKTKLFF | 1 | DV1 | DJ2 |
| (SEQ ID NO: 475) | | | |
| CALGELRSLLHLHWGIRTDKLIF | 1 | DV1 | DJ1 |
| (SEQ ID NO: 476) | | | |
| CALGERFRGYWGIQYTDKLIF | 1 | DV1 | DJ1 |
| (SEQ ID NO: 477) | | | |
| CALGERHPSYWGNKGHTDKLIF | 1 | DV1 | DJ1 |
| (SEQ ID NO: 478) | | | |
| CALGFPPVLGDPYTDKLIF | 1 | DV1 | DJ1 |
| (SEQ ID NO: 479) | | | |
| CALGGSGISYVGILGKLIF | 1 | DV1 | DJ1 |
| (SEQ ID NO: 480) | | | |
| CALGGTSYVPWGIVRRDKLIF | 1 | DV1 | DJ1 |
| (SEQ ID NO: 481) | | | |
| CALGKGGNGVYWGSTRPLIF | 1 | DV1 | DJ4 |
| (SEQ ID NO: 482) | | | |
| CALGNEAFRLVLGETDKLIF | 1 | DV1 | DJ1 |
| (SEQ ID NO: 483) | | | |
| CALGPLSTPPYWGILGTDKLIF | 1 | DV1 | DJ1 |
| (SEQ ID NO: 484) | | | |
| CALGPRFLRGVVGIRYTDKLIF | 1 | DV1 | DJ1 |
| (SEQ ID NO: 485) | | | |
| CALGVLPTLGDQGPTDKLIF | 1 | DV1 | DJ1 |
| (SEQ ID NO: 486) | | | |
| CALRGGRSPVLGDTLKRRTDKLIF | 1 | DV1 | DJ1 |
| (SEQ ID NO: 487) | | | |

Patient 1, Challenge 2
PB-IE GD TCRδ (56)

| | | | |
|---|---|---|---|
| CALGGLPTLGDTPTDKLIF | 17 | DV1 | DJ1 |
| (SEQ ID NO: 459) | | | |
| CALGELRSLLHLHWGIRTDKLIF | 4 | DV1 | DJ1 |
| (SEQ ID NO: 476) | | | |
| CALGERSPSYWGPHFTDKLIF | 4 | DV1 | DJ1 |
| (SEQ ID NO: 488) | | | |
| CAASAGGPQTTDKLIF | 2 | DV5 | DJ1 |
| (SEQ ID NO: 489) | | | |
| CALGDGGGFYTSRVLGGYAFVTTDKLIF | 2 | DV1 | DJ1 |
| (SEQ ID NO: 462) | | | |
| CALGELVRSYFGIRGGKLIF | 2 | DV1 | DJ1 |
| (SEQ ID NO: 490) | | | |
| CALGERLPNYWGTLYTDKLIF | 2 | DV1 | DJ1 |
| (SEQ ID NO: 491) | | | |
| CALGERRPSYWGIRRGPLIF | 2 | DV1 | DJ4 |
| (SEQ ID NO: 492) | | | |
| CALGGSGISYVGILGKLIF | 2 | DV1 | DJ1 |
| (SEQ ID NO: 480) | | | |
| CAASPLVGNTDKLIF | 1 | DV5 | DJ1 |
| (SEQ ID NO: 493) | | | |
| CACDTVGIQSDKLIF | 1 | DV2 | DJ1 |
| (SEQ ID NO: 494) | | | |
| CALCLLADWGYTDKLIF | 1 | DV1 | DJ1 |
| (SEQ ID NO: 460) | | | |
| CALGDPPSRGARPDKLIF | 1 | DV1 | DJ1 |
| (SEQ ID NO: 495) | | | |
| CALGDPTGPYWGKYYLSYTDKLIF | 1 | DV1 | DJ1 |
| (SEQ ID NO: 496) | | | |
| CALGECHPSYWGRPINTDKLIF | 1 | DV1 | DJ1 |
| (SEQ ID NO: 497) | | | |
| CALGECPTRHPTGGYIPYTDKLIF | 1 | DV1 | DJ1 |
| (SEQ ID NO: 498) | | | |
| CALGEFFPRYWGTTYTDKLIF | 1 | DV1 | DJ1 |
| (SEQ ID NO: 472) | | | |
| CALGELPYWALRGADKLIF | 1 | DV1 | DJ1 |
| (SEQ ID NO: 463) | | | |
| CALGELQPRYWGRRFDKTKLFF | 1 | DV1 | DJ2 |
| (SEQ ID NO: 475) | | | |
| CALGELRPSYVFGGYAYKLIF | 1 | DV1 | DJ1 |
| (SEQ ID NO: 499) | | | |
| CALGELSPRYWGIGYTDKLIF | 1 | DV1 | DJ1 |
| (SEQ ID NO: 500) | | | |
| CALGELSRPADWGILIYTDKLIF | 1 | DV1 | DJ1 |
| (SEQ ID NO: 501) | | | |
| CALGEPTGAFLLTGGFTDKLIF | 1 | DV1 | DJ1 |
| (SEQ ID NO: 502) | | | |
| CALGEQNPRYWGASYTDKLIF | 1 | DV1 | DJ1 |
| (SEQ ID NO: 503) | | | |
| CALGERLPSYWGISYTDKLIF | 1 | DV1 | DJ1 |
| (SEQ ID NO: 504) | | | |
| CALGESYVSYWGGYLYTDKLIF | 1 | DV1 | DJ1 |
| (SEQ ID NO: 505) | | | |
| CALGEYLPRYWGIHGTDKLIF | 1 | DV1 | DJ1 |
| (SEQ ID NO: 506) | | | |
| CALGFPPVLGDPYTDKLIF | 1 | DV1 | DJ1 |
| (SEQ ID NO: 479) | | | |

Table 3f
Patient 3
PB-IE GD TCRδ (115)

| | | | |
|---|---|---|---|
| CALGELPLLGDTLRSYTDKLIF | 5 | DV1 | DJ1 |
| (SEQ ID NO: 507) | | | |
| CALGERGPRYWGIAYTDKLIF | 1 | DV1 | DJ1 |
| (SEQ ID NO: 508) | | | |
| CALGVPLLQVKLGDTKGLLGDTDKLIF | 3 | DV1 | DJ1 |
| (SEQ ID NO: 509) | | | |
| CALGERFPWPHTDKLIF | 1 | DV1 | DJ1 |
| (SEQ ID NO: 510) | | | |
| CALGNIWGVTDKLIF | 3 | DV1 | DJ1 |
| (SEQ ID NO: 511) | | | |
| CALGEPSDSAYWGIRGNTDKLIF | 1 | DV1 | DJ1 |
| (SEQ ID NO: 512) | | | |
| CALGELVLRYWGGRMDKLIF | 3 | DV1 | DJ1 |
| (SEQ ID NO: 513) | | | |
| CALGEPRAVLGDTLGDKLIF | 1 | DV1 | DJ1 |
| (SEQ ID NO: 514) | | | |
| CALGEGAGILTGDKLIF | 3 | DV1 | DJ1 |
| (SEQ ID NO: 515) | | | |
| CALGEPLPSYWGPRGSDKLIF | 1 | DV1 | DJ1 |
| (SEQ ID NO: 516) | | | |
| CAFLRIRPDKLIF | 3 | DV1 | DJ1 |
| (SEQ ID NO: 517) | | | |
| CALGEPDSTFVRGGYAGNTDKLIF | 1 | DV1 | DJ1 |
| (SEQ ID NO: 518) | | | |
| CALVSNPPPRYPGVRDTDKLIF | 2 | DV1 | DJ1 |
| (SEQ ID NO: 519) | | | |
| CALGEPDLPTTWYTDKLIF | 1 | DV1 | DJ1 |
| (SEQ ID NO: 520) | | | |
| CALVADYWGIGTDKLIF | 2 | DV1 | DJ1 |
| (SEQ ID NO: 521) | | | |
| CALGEPALQLGVNKLIF | 1 | DV1 | DJ1 |
| (SEQ ID NO: 522) | | | |
| CALGPRFLRRGIRADKLIF | 2 | DV1 | DJ1 |
| (SEQ ID NO: 523) | | | |
| CALGENPPPYLGGYPYTDKLIF | 1 | DV1 | DJ1 |
| (SEQ ID NO: 524) | | | |
| CALGPRAHQRTGDRVTAQLFF | 2 | DV1 | DJ2 |
| (SEQ ID NO: 525) | | | |
| CALGENFPSSWGIHRYTDKLIF | 1 | DV1 | DJ1 |
| (SEQ ID NO: 526) | | | |
| CALGNPKGTSYGLRGIPPYTDKLIF | 2 | DV1 | DJ1 |
| (SEQ ID NO: 527) | | | |
| CALGELVYPGGYYGRETAQLFF | 1 | DV1 | DJ2 |
| (SEQ ID NO: 528) | | | |
| CALGKGGSYVVHFYWGIESTDKLIF | 2 | DV1 | DJ1 |
| (SEQ ID NO: 529) | | | |
| CALGELVFLLRAGLIF | 1 | DV1 | DJ1 |
| (SEQ ID NO: 530) | | | |
| CALGISLLRLGDMISDKLIF | 2 | DV1 | DJ1 |
| (SEQ ID NO: 531) | | | |
| CALGELSDLQCVLGDRPTRPLIF | 1 | DV1 | DJ4 |
| (SEQ ID NO: 532) | | | |
| CALGEPPFLRRYRYTDKLIF | 2 | DV1 | DJ1 |
| (SEQ ID NO: 533) | | | |
| CALGELRRIYWGIRIDKLIF | 1 | DV1 | DJ1 |
| (SEQ ID NO: 534) | | | |
| CALGELPTWTYWGIDKLIF | 2 | DV1 | DJ1 |
| (SEQ ID NO: 535) | | | |
| CALGELLPRYWGIGGTDKLIF | 1 | DV1 | DJ1 |
| (SEQ ID NO: 536) | | | |
| CALGELIRGYWGIRYTDKLIF | 2 | DV1 | DJ1 |
| (SEQ ID NO: 537) | | | |
| CALGELLASETYTGGSVVLYRARKTDKLIF | 1 | DV1 | DJ1 |
| (SEQ ID NO: 538) | | | |
| CALGDPPPHRSLLYRYKLIF | 2 | DV1 | DJ1 |
| (SEQ ID NO: 539) | | | |
| CALGEKGNMPLGDIIDKLIF | 1 | DV1 | DJ1 |
| (SEQ ID NO: 540) | | | |
| CALGALPTLGDRGVDKLIF | 2 | DV1 | DJ1 |
| (SEQ ID NO: 541) | | | |
| CALGEHEVHPGGYWYTDKLIF | 1 | DV1 | DJ1 |
| (SEQ ID NO: 542) | | | |
| CALDTPKHSSGGYFKRTDKLIF | 2 | DV1 | DJ1 |
| (SEQ ID NO: 543) | | | |
| CALGEGTGDFGRWGILVYTDKLIF | 1 | DV1 | DJ1 |
| (SEQ ID NO: 544) | | | |
| CASHFLRAGYAKLIF | 1 | DV3 | DJ1 |
| (SEQ ID NO: 545) | | | |
| CALGEGPFLRTGGLYTDKLIF | 1 | DV1 | DJ1 |
| (SEQ ID NO: 546) | | | |
| CASCPGVGDNDKLIF | 1 | DV3 | DJ1 |
| (SEQ ID NO: 547) | | | |
| CALGEGPAPIWGIRRRSYTDKLIF | 1 | DV1 | DJ1 |
| (SEQ ID NO: 548) | | | |
| CAQTTYWGMGGQYTDKLIF | 1 | DV3 | DJ1 |
| (SEQ ID NO: 549) | | | |
| CALGEFYWGILSDKLIF | 1 | DV1 | DJ1 |
| (SEQ ID NO: 550) | | | |
| CAPFSWPDKLIF | 1 | DV3 | DJ1 |
| (SEQ ID NO: 551) | | | |
| CALGEEIPTGGYPDKLIF | 1 | DV1 | DJ1 |
| (SEQ ID NO: 552) | | | |
| CALVSGGFPSYADKLIF | 1 | DV1 | DJ1 |
| (SEQ ID NO: 553) | | | |
| CALGEDPSFLRLGIRYTDKLIF | 1 | DV1 | DJ1 |
| (SEQ ID NO: 554) | | | |
| CALRWGIRGADKLIF | 1 | DV3 | DJ1 |
| (SEQ ID NO: 555) | | | |
| CALGDWRSSYFNWGISSPDKLIF | 1 | DV1 | DJ1 |
| (SEQ ID NO: 556) | | | |
| CALRVFTYWGDTDKLIF | 1 | DV3 | DJ1 |
| (SEQ ID NO: 557) | | | |
| CALGDPSEEAHTGGYNTDKLIF | 1 | DV1 | DJ1 |
| (SEQ ID NO: 558) | | | |
| CALGVRIFPPSLLGDTGYGGVLIF | 1 | DV1 | DJ1 |
| (SEQ ID NO: 559) | | | |
| CALGDLLGLPRGPTDKLIF | 1 | DV1 | DJ1 |
| (SEQ ID NO: 560) | | | |
| CALGSLLINWGIVTDKLIF | 1 | DV1 | DJ1 |
| (SEQ ID NO: 561) | | | |
| CALGDFPTWGGVPDKLIF | 1 | DV1 | DJ1 |
| (SEQ ID NO: 562) | | | |
| CALGSGAYPYRTGGRELIF | 1 | DV1 | DJ1 |
| (SEQ ID NO: 563) | | | |
| CALGASLGDNSPDKLIF | 1 | DV1 | DJ1 |
| (SEQ ID NO: 564) | | | |
| CALGPPPFLIGSWDTRQMFF | 1 | DV1 | DJ1 |
| (SEQ ID NO: 565) | | | |
| CALGALGSLPTHWGIRATDKLIF | 1 | DV1 | DJ1 |
| (SEQ ID NO: 566) | | | |
| CALGPGAFLRSWGQKLIF | 1 | DV1 | DJ1 |
| (SEQ ID NO: 567) | | | |
| CALGALGLRGSLGVYRKLIF | 1 | DV1 | DJ1 |
| (SEQ ID NO: 568) | | | |
| CALGNSYWGIPYTDKLIF | 1 | DV1 | DJ1 |
| (SEQ ID NO: 569) | | | |
| CALEAPYTDKLIF | 1 | DV1 | DJ1 |
| (SEQ ID NO: 570) | | | |
| CALGNHWADKLIF | 1 | DV1 | DJ1 |
| (SEQ ID NO: 571) | | | |
| CALAQPSSNLLIHWGILDKLIF | 1 | DV1 | DJ1 |
| (SEQ ID NO: 572) | | | |
| CALGLPIGLGDSYLYKLIF | 1 | DV1 | DJ1 |
| (SEQ ID NO: 573) | | | |
| CAITGSKGTDKLIF | 1 | DV3 | DJ1 |
| (SEQ ID NO: 574) | | | |
| CALGKRPYPLYWGIRGYTDKLIF | 1 | DV1 | DJ1 |
| (SEQ ID NO: 575) | | | |
| CAFRHGPNYPLIYWGISKLIF | 1 | DV3 | DJ1 |
| (SEQ ID NO: 576) | | | |
| CALGFYWGEYTDKLIF | 1 | DV1 | DJ1 |
| (SEQ ID NO: 577) | | | |
| CAFRGLWGYTDKLIF | 1 | DV3 | DJ1 |
| (SEQ ID NO: 578) | | | |
| CALGEYSRLTGVYTDKLIF | 1 | DV1 | DJ1 |
| (SEQ ID NO: 579) | | | |
| CAFPXWGHSLYTDKLIF | 1 | DV3 | DJ1 |
| (SEQ ID NO: 580) | | | |
| CALGEYPPLGDTFVXTTXDTRQMFF | 1 | DV1 | DJ3 |
| (SEQ ID NO: 581) | | | |
| CAFLALPMYTDKLIF | 1 | DV3 | DJ1 |
| (SEQ ID NO: 582) | | | |
| CALGEWFPGYFLTKFRNTDKLIF | 1 | DV1 | DJ1 |
| (SEQ ID NO: 583) | | | |
| CACVKAFLKRGDTPYTDKLIF | 1 | DV2 | DJ1 |
| (SEQ ID NO: 584) | | | |
| CALGESVRWVFGEYTDKLIF | 1 | DV1 | DJ1 |
| (SEQ ID NO: 585) | | | |
| CACTFLGLGGSNTDKLIF | 1 | DV3 | DJ1 |
| (SEQ ID NO: 585) | | | |
| CALGERYPKYWGAPGTDKLIF | 1 | DV1 | DJ1 |
| (SEQ ID NO: 587) | | | |
| CACETWGIKGTDKLIF | 1 | DV2 | DJ1 |
| (SEQ ID NO: 588) | | | |
| CALGERSYVPYWGTGRGTDKLIF | 1 | DV1 | DJ1 |
| (SEQ ID NO: 589) | | | |
| CACERGGYAFTDKLIF | 1 | DV2 | DJ1 |
| (SEQ ID NO: 590) | | | |
| CALGERIPTSWGIXYTDKLIF | 1 | DV1 | DJ1 |
| (SEQ ID NO: 591) | | | |
| CACDSRTSTWGIRMADKLIF | 1 | DV2 | DJ1 |
| (SEQ ID NO: 592) | | | |

Table 3g
Patient 4
PB-IE GD TCRδ (60)

| | | | |
|---|---|---|---|
| CALGELPPPGGYFDKLIF | 3 | DV1 | DJ1 |
| (SEQ ID NO: 593) | | | |
| CAFKGLLGGSVGLIF | 2 | DV3 | DJ1 |
| (SEQ ID NO: 594) | | | |
| CALGDSSLGGWGILSSTDKLIF | 2 | DV1 | DJ1 |
| (SEQ ID NO: 595) | | | |
| CALGVLHWGNSLTAQLFF | 2 | DV1 | DJ2 |
| (SEQ ID NO: 596) | | | |
| CALPFSYWGIRLVGTDKLIF | 2 | DV3 | DJ1 |
| (SEQ ID NO: 597) | | | |
| CASTGAVGKSPKLIF | 2 | DV3 | DJ1 |
| (SEQ ID NO: 598) | | | |
| CAASAGLPGGLGYTDKLIF | 1 | DV5 | DJ1 |
| (SEQ ID NO: 599) | | | |
| CAASALRGSFDKLIF | 1 | DV5 | DJ1 |
| (SEQ ID NO: 600) | | | |
| CACDHDYGTGGVRKLIF | 1 | DV2 | DJ1 |
| (SEQ ID NO: 601) | | | |
| CACRLPTRWGIGYTDKLIF | 1 | DV3 | DJ1 |
| (SEQ ID NO: 602) | | | |
| CACRPSYGGIVKLIF | 1 | DV3 | DJ1 |
| (SEQ ID NO: 603) | | | |
| CAFILTIYGPGGITDKLIF | 1 | DV3 | DJ1 |
| (SEQ ID NO: 604) | | | |
| CAFPTGGLLGDTDKLIF | 1 | DV3 | DJ1 |
| (SEQ ID NO: 605) | | | |
| CAFVGGPYTDKLIF | 1 | DV3 | DJ1 |
| (SEQ ID NO: 606) | | | |
| CALADLRPGGYSAQLFF | 1 | DV1 | DJ2 |
| (SEQ ID NO: 607) | | | |
| CALEVVHHPIRYTDKLIF | 1 | DV1 | DJ1 |
| (SEQ ID NO: 608) | | | |
| CALGAHLRNYWGPLYTDKLIF | 1 | DV1 | DJ1 |
| (SEQ ID NO: 609) | | | |
| CALGAYPPGGTGRYTDKLIF | 1 | DV1 | DJ1 |
| (SEQ ID NO: 610) | | | |
| CALGDFLPSYWGIRGTDKLIF | 1 | DV1 | DJ1 |
| (SEQ ID NO: 611) | | | |
| CALGDPFQNYQGPYTDKLIF | 1 | DV1 | DJ1 |
| (SEQ ID NO: 612) | | | |
| CALGEAFLSYWGTNHDKLIF | 1 | DV1 | DJ1 |
| (SEQ ID NO: 613) | | | |
| CALGEGGGVLRNPYTDKLIF | 1 | DV1 | DJ1 |
| (SEQ ID NO: 614) | | | |
| CALGEHGAAFLPYWGIRRGKLIF | 1 | DV1 | DJ1 |
| (SEQ ID NO: 615) | | | |
| CALGEIYRGYWGIRAGDKLIF | 1 | DV1 | DJ1 |
| (SEQ ID NO: 616) | | | |
| CALGELHWGTRYTDKLIF | 1 | DV1 | DJ1 |
| (SEQ ID NO: 617) | | | |
| CALGELLRTGGLAQLFF | 1 | DV1 | DJ2 |
| (SEQ ID NO: 618) | | | |
| CALGELMLGRWGEYTDKLIF | 1 | DV1 | DJ1 |
| (SEQ ID NO: 619) | | | |
| CALGELNLPQYWGPLVGTDKLIF | 1 | DV1 | DJ1 |
| (SEQ ID NO: 620) | | | |
| CALGELPPWGIPYTDKLIF | 1 | DV1 | DJ1 |
| (SEQ ID NO: 621) | | | |
| CALGELRLRWMGDTLFLQLTDKLIF | 1 | DV1 | DJ1 |
| (SEQ ID NO: 622) | | | |
| CALGELRRGIRGQRIGTDKLIF | 1 | DV1 | DJ1 |
| (SEQ ID NO: 623) | | | |
| CALGELSRPSYYYDPSYTDKLIF | 1 | DV1 | DJ1 |
| (SEQ ID NO: 624) | | | |
| CALGELSSPHTGGYYTDKLIF | 1 | DV1 | DJ1 |
| (SEQ ID NO: 625) | | | |
| CALGELSYRGGWGIRADKLIF | 1 | DV1 | DJ1 |
| (SEQ ID NO: 626) | | | |
| CALGENKFVFGGLIVLTAQLFF | 1 | DV1 | DJ2 |
| (SEQ ID NO: 627) | | | |
| CALGEPIGPPLLGVYTDKLIF | 1 | DV1 | DJ1 |
| (SEQ ID NO: 628) | | | |
| CALGEPQTFLPRYWGGTYTDKLIF | 1 | DV1 | DJ1 |
| (SEQ ID NO: 629) | | | |
| CALGEPSTGGSDKLIF | 1 | DV1 | DJ1 |
| (SEQ ID NO: 630) | | | |
| CALGEQWILRGDTDKLIF | 1 | DV1 | DJ1 |
| (SEQ ID NO: 631) | | | |
| CALGERLRGYALKTDKLIF | 1 | DV1 | DJ1 |
| (SEQ ID NO: 632) | | | |
| CALGERLSPYYTDKLIF | 1 | DV1 | DJ1 |
| (SEQ ID NO: 633) | | | |
| CALGERPSYGWGFGWTDKLIF | 1 | DV1 | DJ1 |
| (SEQ ID NO: 634) | | | |
| CALGETTLSYWGIRYTDKLIF | 1 | DV1 | DJ1 |
| (SEQ ID NO: 635) | | | |
| CALGGGLPTSGGYRSYTDKLIF | 1 | DV1 | DJ1 |
| (SEQ ID NO: 636) | | | |
| CALGHRAPSRAQPYWGILAYTDKLIF | 1 | DV1 | DJ1 |
| (SEQ ID NO: 637) | | | |
| CALGKPAKSYWGMRYTDKLIF | 1 | DV1 | DJ1 |
| (SEQ ID NO: 638) | | | |
| CALGPLPGGYSSWDTRQMFF | 1 | DV1 | DJ3 |
| (SEQ ID NO: 639) | | | |
| CALGQRIPSYWGIAGSTDKLIF | 1 | DV1 | DJ1 |
| (SEQ ID NO: 640) | | | |
| CALGVISPSYWGPQYTDKLIF | 1 | DV1 | DJ1 |
| (SEQ ID NO: 641) | | | |
| CALGVSSSAGDLLTDKLIF | 1 | DV1 | DJ1 |
| (SEQ ID NO: 642) | | | |
| CALKPGGYSLTDKLIF | 1 | DV1 | DJ1 |
| (SEQ ID NO: 643) | | | |
| CALMAGPYTDKLIF | 1 | DV3 | DJ1 |
| (SEQ ID NO: 644) | | | |
| CASVCYGNGHISRLDKLIF | 1 | DV3 | DJ1 |
| (SEQ ID NO: 645) | | | |

TABLE 4
Primers used for TCRδ sequencing: TCRδ was
amplified using a series of nested PCR
reactions.

TRDV primers, reaction 1:
TRDV1
CCAGGGTTCTGATGAACAGAATGC
(SEQ ID NO: 646)
TRDV2
CCTGGTTTCAAAGACAATTTCCAAG
(SEQ ID NO: 647)
TRDV3
GGATAACAGCAGATCAGAAGGTGC
(SEQ ID NO: 648)
TRDV4
GCAAAATGCAACAGAAGGTCGCTA
(SEQ ID NO: 649)
TRDV5
GGATAAAAATGAAGATGGAAGATTCAC
(SEQ ID NO: 650)
TRDV6
CCAGATGTGAGTGAAAAGAAAGAAG
(SEQ ID NO: 651)
TRDV7
GCTAACTTCAAGTGGAATTGAAAAGA
(SEQ ID NO: 652)
TRDV8
GAAGCTTATAAGCAACAGAATGCAAC
(SEQ ID NO: 653)
TRDV primers, reaction 2:
TRDV1
GCATACGAGCTCTTCCGATCTGAGTGGTCGCTATTCTGTCAACTTCAA
(SEQ ID NO: 654)
TRDV2
GCATACGAGCTCTTCCGATCTGAGTGACATTGATATTGCAAAGAACCTG
(SEQ ID NO: 655)
TRDV3
GCATACGAGCTCTTCCGATCTGAGGACGGTTTTCTGTGAAACACATTC
(SEQ ID NO: 656)
TRDV4
GCATACGAGCTCTTCCGATCTGATCCAGAAGGCAAGAAAATCCGCCA
(SEQ ID NO: 657)
TRDV5
GCATACGAGCTCTTCCGATCTGACTTAAACAAAAGTGCCAAGCACCTC
(SEQ ID NO: 658)
TRDV6
GCATACGAGCTCTTCCGATCTGACACAATCTCCTTCAATAAAAGTGCCA
(SEQ ID NO: 659)
TRDV7
GCATACGAGCTCTTCCGATCTGAGGAAGACTAAGTAGCATATTAGATAAG
(SEQ ID NO: 660)
TRDV8
GCATACGAGCTCTTCCGATCTGACTGTGAACTTCCAGAAAGCAGCCA
(SEQ ID NO: 661)
5′ primer, reaction 3:
GCATACGAGCTCTTCCGATCTGA
(SEQ ID NO: 662)
TRDC primer, reaction 1:
CGAGATTTATTCTTATATCCTTGGGG
(SEQ ID NO: 663)
TRDC primer, reaction 2:
CCTTCACCAGACAAGCGACATTTG
(SEQ ID NO: 664)
TRDC primer, reaction 3:
CATTTTTCATGACAAAAACGGATGGT
(SEQ ID NO: 665)

While the preferred embodiments of the invention have been illustrated and described, it will be appreciated that various changes can be made therein without departing from the spirit and scope of the invention.

1. A method for diagnosing celiac disease in a subject, the method comprising:
a) obtaining a blood sample comprising peripheral blood lymphocytes from the subject after the subject has consumed gluten for 1 to 3 days; and
b) measuring the levels of activated, gut-bound CD8+ αβ T lymphocytes and γδ T lymphocytes in the blood sample, wherein increased levels of activated, gut-bound CD8+αβ T lymphocytes and γδ T lymphocytes compared to the levels of activated, gut-bound CD8+αβ T lymphocytes and γδ T lymphocytes in a control sample indicate that the subject has celiac disease.

2. The method of claim 1, wherein activated, gut-bound CD8+ αβ T lymphocytes and γδ T lymphocytes are identified by detection of the activation marker, CD38, and the intestinal homing markers, CD103 and β7 integrin.

3. The method of claim 1, wherein the blood sample is obtained from the subject up to 6 days after the subject consumes gluten.

4. The method of claim 1, wherein the subject consumes one or more doses of a gluten containing food, powder, or pill.

5. The method of claim 1, wherein the control sample comprises peripheral blood lymphocytes obtained from the subject after the subject has not consumed gluten for a period of at least 2 weeks.

6. The method of claim 5, wherein the control sample comprises peripheral blood lymphocytes obtained from the subject after the subject has not consumed gluten for a period of at least one month.

7. The method of claim 1, wherein the control sample comprises peripheral blood lymphocytes obtained from a healthy subject who does not have celiac disease.

8. The method of claim 1, further comprising comparing the levels of CD8+ αβ T lymphocytes and γδ T lymphocytes to reference levels for one or more normal subjects.

9. The method of claim 1, further comprising comparing the levels of CD8+ αβ T lymphocytes and γδ T lymphocytes to reference levels for one or more subjects who have celiac disease.

10. The method of claim 1, wherein measuring the levels of CD8+ αβ T lymphocytes and γδ T lymphocytes comprises counting cells using a flow cytometer, Coulter counter, CASY counter, hemocytometer, or microscopic imaging.

11. The method of claim 1, further comprising detecting an increase in the levels of CD8+ αβ T lymphocytes or γδ T lymphocytes expressing one or more cell markers selected from the group consisting of αE (CD103), β7 integrin, and CD38 compared to the levels of the T lymphocytes expressing the one or more cell markers in a control sample.

12. The method of claim 1, further comprising detecting an increased number of CD4⁺ T cells.

13. The method of claim 1, further comprising detecting one or more cellular markers.

14. The method of claim 13, wherein one or more cellular markers are detected by a method selected from the group consisting of immunofluorescent antibody assay (IFA), enzyme-linked immuno-culture assay (ELICA), flow cytometry, cytometry by time-of-flight (CyTOF), and magnetic cell sorting.

15. The method of claim 13, wherein one or more cellular markers selected from the group consisting of CD38, CD45RO, CD27, CD28, CD62L, and CCR7 are detected on a CD8+αβ T cell.

16. The method of claim 15, comprising counting the number of CD8+ T cells having a phenotype of CD38⁺, CD45RO⁺, CD27⁻, CD28^{low}, CD62L⁻, and CCR7^{low}, wherein an increase in the number of CD8+ T cells having said phenotype compared to a control sample indicates that the subject has celiac disease.

17. The method of claim 13, wherein one or more cellular markers selected from the group consisting of CD45RO and CD27 are detected on a γδ T cell.

18. The method of claim 17, comprising counting the number of γδ T cells having a phenotype of CD45RO⁺ and CD27⁻, wherein an increase in the number of γδ T cells having said phenotype compared to a control sample indicates that the subject has celiac disease.

19. The method of claim 1, further comprising detecting activation of a CD8+αβ T lymphocyte or γδ T lymphocyte.

20. The method of claim 19, wherein detecting activation comprises performing an enzyme-linked immunosorbent spot (ELISPOT) assay, a T cell proliferation assay, flow cytometry, or CyTOF.

21. The method of claim 20, wherein secretion of one or more secretory molecules selected from the group consisting of IFN-γ, TNF-α, TNF-β, IL-2, IL-3, Fas ligand, perforin, or a granzyme is detected by the ELISPOT assay.

22. The method of claim 20, wherein one or more cellular markers selected from the group consisting of CD38, a natural killer (NK) receptor, CD45RO, and CD27 are detected by flow cytometry or CyTOF.

23. The method of claim 1, wherein the subject is a human being.

24. The method of claim 1, further comprising diagnosing the subject with irritable bowel syndrome.

25. A method for treating a subject suspected of having celiac disease the method comprising:
a) diagnosing celiac disease in the subject according to the method of claim 1; and
b) treating the subject with a gluten-free diet if increased levels of activated, gut-bound CD8+αβ T lymphocytes and γδ T lymphocytes compared to the levels of activated, gut-bound CD8+αβ T lymphocytes and γδ T lymphocytes in a control sample indicate that the subject has celiac disease.

26. The method of claim 25, further comprising measuring the levels of activated, gut-bound CD8+αβ T lymphocytes and γδ T lymphocytes after treating the subject with a gluten-free diet and comparing to reference levels for gut-bound CD8+αβ T lymphocytes and γδ T lymphocytes.

27-46. (canceled)
